(19) United States
(12) Patent Application Publication
(10) Pub. No.: US 2026/0337212 A1
(43) Pub. Date: Oct. 29, 2026

(54) IP4-4,6 SUBSTITUTED DERIVATIVE COMPOUNDS FOR USE IN THE TREATMENT, INHIBITION OF PROGRESSION, AND PREVENTION OF ECTOPIC CALCIFICATION

(71) Applicant: SANIFIT THERAPEUTICS, S.A., Palma de Mallorca (ES)

(72) Inventors: Mohamad Firas BASSISSI, Palma de Mallorca (ES); Marc BLASCO FERRER, Palma de Mallorca (ES); María del Mar PÉREZ FERRER, Palma de Mallorca (ES); Miquel David FERRER REYNÉS, Palma de Mallorca (ES); Carolina SALCEDO ROCA, Palma de Mallorca (ES); Alexander M. GOLD, Palma de Mallorca (ES); Joan PERELLÓ BESTARD, Palma de Mallorca (ES)

(21) Appl. No.: 19/099,414

(22) PCT Filed: Jul. 31, 2023
(86) PCT No.: PCT/EP2023/071128

§ 371 Date: Jan. 28, 2025

(30) Foreign Application Priority Data
Jul. 29, 2022 (EP) 22382738.7

(51) Int. Cl. C07F 9/117; A61K 9/00; A61K 31/6615; A61P 9/00; A61P 17/00; A61P 43/00

(52) U.S. Cl. CPC C07F 9/117; A61K 9/0019; A61K 31/6615; A61P 9/00; A61P 17/00; A61P 43/00

(57) ABSTRACT

The present invention provides IP4-4,6 substituted derivatives for use in the prevention, inhibition of progression, and treatment of (a) ectopic calcification and the consequences thereof, and (b) a disease and/or condition related to ectopic calcification and the consequences thereof. Also provided are methods, pharmaceutical compositions and formulations, methods of use, articles of manufacture, and kits for use in the prevention, inhibition of progression, and treatment of (a) ectopic calcification and the consequences thereof, and (b) a disease and/or condition related to ectopic calcification and the consequences thereof.

### TECHNICAL FIELD

The present invention relates to IP4-4,6 substituted derivative compounds for use in the treatment, inhibition of progression, and prevention of (a) ectopic calcification and (b) diseases and/or conditions related to ectopic calcification. Pharmaceutical and kits for such uses are also provided.

### STATE OF THE ART

Ectopic calcification (EC) is defined as inappropriate biomineralization occurring in soft tissues (Cotran R, et al., Pathological Basis of Disease, 5th Ed., Robbins S, et al., Eds. (WB Saunders, Philadelphia, PA, US, 1994, pp 1-35). EC is typically composed of calcium phosphate salts, including hydroxyapatite (HAP), but can also consist of calcium oxalates and octacalcium phosphate as seen in kidney stones (Pak C, Am J Kidney Dis 1991; 18:624-632). EC may manifest independently on different locations, although it develops through similar mechanisms to physiological calcification (Giachelli C, Am J Pathol. 1999; 154(3):671-67). The diversity of types of EC are believed to be related with having a chronic or a rare disease. See FIG. 1 (Kempf H, et al., Front Cell Dev Biol. 2021; 9:759702).

Thus, EC can be found in soft tissues such as vessels, heart valves, lungs, kidney, and brain. This pathological calcification leads to organ damage. Some of the most relevant pathologies associated to EC are listed below. See Table A.

TABLE A

| | |
|---|---|
| 1 | pseudoxanthoma elasticum (PXE) |
| 2 | general arterial calcification of infancy (GACI) |
| 3 | calcinosis cutis |
| 4 | primary familial brain calcification (PFBC) |
| 5 | idiopathic brain calcification (Fahr's disease) |
| 6 | calcification of joints and arteries (CALJA) |
| 7 | fibrodysplasia ossificans progressiva (FOP) |
| 8 | calcific aortic valve stenosis (CAVS) |
| 9 | calcium pyrophosphate deposition disease (CPPD) |
| 10 | calcific band keratopathy (CBK) |
| 11 | primary hyperoxaluria (PH) |
| 12 | hyperphosphatemic familial tumoral calcinosis (HFTC) |
| 13 | adrenal and intracranial calcification in familial |
| | cerebral cavernous malformations |
| 14 | age-related macular degeneration (AMD) related to |
| | calcium deposits |
| 15 | calcification of articular cartilage in osteoarthritis |
| 16 | cardiovascular disease linked to aging |
| 17 | peripheral arterial disease (PAD) |
| 18 | calciphylaxis (CUA) |
| 19 | portal vein calcification |
| 20 | Wolman's disease adrenal calcification |
| 21 | soft tissue calcification owing to sarcoidosis |
| 22 | pineal calcification (PGC) |
| 23 | Sjorgen's syndrome parotid glands calcification |
| 24 | calcific tendinitis |
| 25 | dystrophic calcification |
| 26 | metastatic calcification |
| 27 | breast calcification |
| 28 | Nephrolithiasis, renal lithiasis or kidney stones |
| 28 | seminal vesicle calculi |
| 30 | sialolithiasis |
| 31 | calcification of the seminal vesicles |
| 32 | calcification in osteoarthritis |

As listed above, there are more than 30 pathologies associated to EC in the vascular bed. The causes are diverse but can be grouped in 4 categories:
(i) Genetic mutations (e.g., GACI1, CALJA, PH, FOP, PXE),
(ii) Tissue repair response associated with inflammation and/or immunological responses (e.g., calcinosis cutis),
(iii) Metabolic calcium and phosphate balance disruption (e.g., renal disease), and
(iv) Idiopathic.

Recent data suggest that numerous molecules may modulate this process. The discovery of biomarkers for identifying and/or characterizing these molecules is an expanding field of research (Nitschke Y, et al., Am J Hum Genet. 2012; 90(1):25-39).

From the list depicted in Table A, the most important of the extraosseous EC is that produced in the cardiovascular system, specifically in the arteries and heart valves, as it is a strong independent risk factor for increased mortality and cardiovascular events. In arteries, calcification is associated with CAD, atherosclerotic plaque burden, in peripheral vascular disease, and worse outcomes (e.g., increased risk of dissection) following angioplasty. In the heart, the valves are particularly prone to calcification. Degenerative calcific aortic stenosis is currently the most common valvular lesion encountered in clinical cardiology and one of the most difficult to manage. It is estimated that approximately 1-2% of the elderly population suffer from this pathology, which is characterized by encrustation of aortic valve leaflets with apatite mineral deposits and subsequent stiffening, tearing, and mechanical failure. Congenital anomalies, inflammatory changes such as those seen in rheumatic fever, renal disease, and age are all risk factors for aortic valve stenosis.

The definitive treatment for severe symptomatic aortic stenosis is aortic valve replacement. More than 40,000 patients undergo valve replacement annually in the United States. In the case of bioprosthetic valve, failure is most often attributed, to calcification of the graft. In fact, by 10 years, one third of bioprosthetic valves require replacement, increasing to two thirds by 15 years after implantation. The prevalence of aortic stenosis in those aged >75 years in the European Union and the United States is 3.4%, a significant public health problem.

Another important disease related to EC is Peripheral Artery Disease, and is particularly pronounced in the end stage kidney disease (PAD-ESKD). It affects individuals with end stage kidney disease undergoing hemodialysis. PAD-ESKD causes a reduction in mobility, pain in the lower extremities, and can lead to critical limb ischemia, gangrene, and amputation. A key etiological factor of PAD-ESKD is the accelerated deposition of calcium phosphate in the form of HAP in the media and intima of peripheral arteries in the lower extremities. This calcification causes narrowing and stiffening of arteries which results in reduction of arterial blood flow and distal ischemia. Risk factors for PAD-ESKD include older age, diabetes, hypertension, and smoking (Chen J, et al., Nephrol Dial Transplant. 2016; 31(7):1145-1151). The duration of dialysis is an important risk factor for PAD-ESKD and the risk factors of hypertension and diabetes are more prevalent and more difficult to control in patients with ESKD than in the general population (Rajagopalan S et al., Circulation 2006; 114(18):1914-1922). Patients with ESKD have been excluded from prior clinical studies of medical therapies for PAD and thus there are no approved therapies specifically for patients with PAD-ESKD.

With the increasing knowledge of the mechanisms of ectopic calcification, the field is facing two critical challenges: finding useful biomarkers for early detection of EC events, and more importantly, finding effective treatments to block or reverse this pathologic process occurring in both chronic and rare diseases (Kempf, 2021, supra).

Regarding the potential targets for possible therapeutic treatments, little research is found in this field. The potential of omega-3 therapy, for its anti-inflammatory benefits, in treating the inflammation in aortic valve stenosis has been evaluated in mice (Artiach G, et al., Circulation 2020; 142:776-789). A synthetic peptide (GFOGER) modifies protein showed that content of released extracellular vesicles in calcified vascular tissues and osteogenic switching of cultured cells in vitro (Mansour A, et al., Front. Cell Dev. Biol. 2020; 8:58976130). However, these findings need to be confirmed in animal models and eventually in humans.

Thus, future work in the field needs to focus on (i) the development of novel biomarkers to predict the progression of ectopic calcification, (ii) the characterization of new animal models and human pathology associated with ectopic calcification, (iii) the understanding of the yet unknown mechanisms of action of some of the critical regulators of EC mineralization, and most importantly, (iv) the development of effective therapies for various types of “soft” tissue calcification (Kempf, 2021, supra). Thus, there is a need in the art for new chemical entities that may be effective in the treatment and prevention of diseases and conditions related to soft tissue calcification.

### BRIEF SUMMARY OF THE INVENTION

The present invention discloses a compound of general formula I:

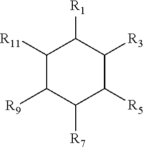

a pharmaceutically acceptable salt thereof, or a combination thereof, wherein
(i) R₁, R₃, R₇, and R₁₁ independently represent OPO₃²⁻; R₅ and R₉ are substituent groups each one corresponding to the formula —O-(alkyl)ₙ-X, wherein n is an integer between 1 and 20, wherein the terminal group X is selected from the group consisting of —H, —OR, —NRR′, —COOR, —CONRR′, —NHCOR, —NHCOOR, —OCONR, —NHSO₂R, —NHCONRR′, halogen, —CF₃, alkyl, alkenyl, alkynyl, carbocycle (saturated or unsaturated), and heterocycle (saturated or unsaturated), and wherein R and R′ are H or an alkyl group;
(ii) R₁, R₃, R₇, and R₁₁ independently represent OPO₃²⁻; R₅ and R₉ are substituent groups each one corresponding to the formula —O-(alkyl)_{y}-Cy-(alkyl)_{y′}-Z, wherein y and y′ are an integer between 0 and 10, wherein Cy is a cyclic linker, wherein the terminal group Z is selected from the group consisting of alkyl, —COR, —OR, —NRR′, —COOR, —CONRR′, —NHCOR, —NHCOOR, —OCONR—NHSO₂R, —NHCONRR′, halogen, and —CF₃, and wherein R and R′ are H or an alkyl group;
(iii) R₁, R₃, R₇, and R₁₁ independently represent OPO₃²⁻; R₅ and R₉ are substituent groups each one corresponding to the formula —O-(alkyl)_{y}-A-(alkyl)_{y′}-Z′, wherein y and y′ are an integer between 0 and 10, wherein A is a linker selected from the group consisting of —CONR—, —NHCOO—, —NHSO₂, —NHCONR—, —NHCO—, and —OCONR, wherein the terminal group Z′ is selected from the group consisting of —OR, —NRR′, —COOR, —CONRR′, —NHCOR, —NHCOOR, —OCONR, —NHSO₂R, —NHCONRR′, carbocycle (saturated or unsaturated), heterocycle (saturated or unsaturated), and wherein R and R′ are H or an alkyl group; and
(iv) the compound of formula I is an analog of (i), (ii) or (iii) wherein at least one of R₁, R₃, R₇ or R₁₁ is thiophosphate (—OPSO₂²⁻) for use in the treatment, inhibition of progression, and prevention of ectopic calcification or the consequences thereof in a subject in need thereof.

In some aspects, the R₅ and R₉ substituent groups of the compound of formula I are identical. In some aspects, the R₅ and R₉ substituent groups of the compound of formula I are different.

In some aspects, the alkyl moiety in the R₅ and R₉ substituent groups includes at least one double or triple carbon bond, i.e., forming an alkenyl or alkynyl chain, respectively. In some aspects, the alkenyl chains in the R₅ and R₉ substituent groups are identical. In some aspects, the alkenyl chains in the R₅ and R₉ substituent groups are different. In some aspects, the alkynyl chains in the R₅ and R₉ substituent groups are identical. In some aspects, the alkynyl chains in the R₅ and R₉ substituent groups are different. In some aspects, the alkyl moiety in the R₅ and R₉ substituent groups includes at least one double carbon bond and at least one triple carbon bond, forming a combination of an alkenyl and an alkynyl chain. In some aspects, the combination of the alkenyl and the alkynyl chains in the R₅ and R₉ substituent groups is identical. In some aspects, the combination of the alkenyl and the alkynyl chains in the R₅ and R₉ substituent groups is different.

In some aspects, the compound of formula I is selected from the group consisting of Compounds 1 to Compound 53.

In some aspects, the pharmaceutically acceptable salt is a sodium salt. In some aspects, the sodium salt is a tetrasodium salt, pentasodium salt, hexasodium salt, heptasodium salt, octasodium salt, nonasodium salt or decasodium salt. In some aspects, the sodium salt is a tetrasodium salt, pentasodium salt, hexasodium salt, heptasodium salt or octasodium salt. In some aspects, the sodium salt is a hexasodium salt, octasodium salt, nonasodium salt or decasodium salt.

The invention also provides a pharmaceutical composition comprising a compound of formula I disclosed above and at least one pharmaceutically acceptable excipient or carrier.

Also provided is a compound or pharmaceutical composition according to the invention for use in the treatment, inhibition of progression, and prevention of a disease and/or condition related to ectopic calcification or the consequences thereof in a subject in need thereof.

In some aspects, the subject is human. In some aspects, the administration is topical, enteral or parenteral. In some aspects, the parenteral administration is intravenous. In some aspects, the intravenous administration is by intravenous infusion.

The present invention also provides a kit or article of manufacture comprising at least one compound of formula I or a pharmaceutical composition comprising a compound of formula I and instructions for administration according to uses disclosed herein. In some aspects, the kit or article of manufacture may also comprise at least one compound selected from the group consisting of the compounds listed in Table 1.

### BRIEF DESCRIPTION OF THE DRAWINGS

FIG. 1 is a simplified schematic representation of the cellular, extracellular, and systemic alterations related to ectopic calcification and their connection to some chronic ana rare diseases.

FIG. 2 is a simplified schematic representation of the physiochemical mechanism of action of the IP4-4,6 substituted derivatives of the present invention inhibiting the crystallization of hydroxyapatite (HAP) and thus being useful in the therapy of HAP-crystallization mediated diseases.

FIG. 3A and FIG. 3B present representative structures of Family A IP4-4,6 substituted derivative compounds (e.g., Compounds 1 to 30, and Compound 52 to 53).

FIG. 4A and FIG. 4B present representative structures of Family B IP4-4,6 substituted derivative compounds (e.g., Compounds 31 to 44).

FIG. 5 presents representative structures of Family C IP4-4,6 substituted derivative compounds (e.g., Compounds 45 to 49).

FIG. 6 presents representative structures of Family D IP4-4,6 substituted derivative compounds (e.g., Compounds 50 to 51).

FIG. 7 is a schematic representation of synthesis Scheme 1.

FIG. 8 is a schematic representation of synthesis Scheme 2.

FIG. 9 is a schematic representation of synthesis Scheme 3.

FIG. 10 is a schematic representation of synthesis Scheme 4.

FIG. 11 is a schematic representation of synthesis Scheme 5.

FIG. 12 represents the mean calcium content (±SD) in Sham rats (no VitD3 treatment) and rats treated with vehicle or Compound 27 (5, 15, and 45 mg/kg) in (A) aorta, (B) heart, (C) femoral artery, (D) carotid arteries, and (E) kidney. For femoral and carotid arteries, the mean of the calcium content in left and right vessels was used. Statistical analysis: One-way ANOVA with Fisher's LSD test for post-hoc comparisons. (#) indicates significant differences vs. sham, (*) indicates significant differences vs vehicle, (§) indicates significant differences vs Compound 27 5 mg/kg (only applies to other Compound 27 doses), (¥) indicates significant differences vs Compound 27 15 mg/kg (only applies to Compound 27 45 mg/kg), p<0.05. N=7-12 animals/group.

FIG. 13 shows the mean normalized blood perfusion (±SD) in Sham rats (no VitD3 treatment) and rats treated with vehicle, Compound 27 (5, 15, and 45 mg/kg) at D13. Statistical analysis: One-way ANOVA with Fisher's LSD test for post-hoc comparisons. The result of the ANOVA was p<0.0001. (#) indicates significant differences vs sham, (*) indicates significant differences vs vehicle, (§) indicates significant differences vs Compound 27 5 mg/kg (only applies to other Compound 27 doses), p<0.05. N=10-12 animals/group.

FIG. 14 depicts the mean maximum walking distance (±SD) in Sham rats (no VitD3 treatment) and rats treated with vehicle, Compound 27 (5, 15, and 45 mg/kg). Statistical analysis: One-way ANOVA with Fisher's LSD test for post-hoc comparisons. The result of the ANOVA was p<0.0001. (#) indicates significant differences vs. sham, (*) indicates significant differences vs vehicle, (§) indicates significant differences vs Compound 27 5 mg/kg (only applies to other Compound 27 doses), (¥) indicates significant differences vs Compound 27 15 mg/kg (only applies to Compound 27 45 mg/kg), (‡) significant differences vs Compound 27 45 mg/kg, p<0.05.

FIG. 15 shows cultures of aortic valve interstitial cells (VIC) (n=7 from calcified valves donors) kept in basic growth media (control), osteodifferentiation media (Osteodiff) and osteodifferentiation media adding (A) IP6, (B) Compound 1, (C) Compound 3 and (D) Compound 6 at various concentrations (1, 3, 10, 30, and 100 μM) for 3 weeks. Calcification was measured by Alizarin Red staining and quantified spectrophotometrically as relative calcification. Values are shown as dot plots with mean. A one-way ANOVA for parametric distributions (IP6) with DMS post-test statistical analysis was performed. For not normally distributed results (Compound 1, Compound 3, and Compound 6), a nonparametric one-way ANOVA (Kruskal-Wallis test) with Dunn's post-test was applied, p<0.05 was considered statistically significant.

FIG. 16 depicts an analysis of hypermineralization using Alizarin Red staining in 10 dpf abcc6a^{-/-} zebrafish larvae. The larvae were kept in E3-medium (n=20) and E3-medium supplemented with Compound 27 1 mM (n=18) from 3 to 10 dpf. Significant reduction of spinal hypermineralization (black boxes) in the abcc6a^{-/-} arvae after treatment with Compound 27 was observed. Quantification of calcified vertebral bodies in Compound 27-treated and untreated abcc6a^{-/-} larvae through Image J analysis revealed a significance of p<0.001, scale bar 500 μm.

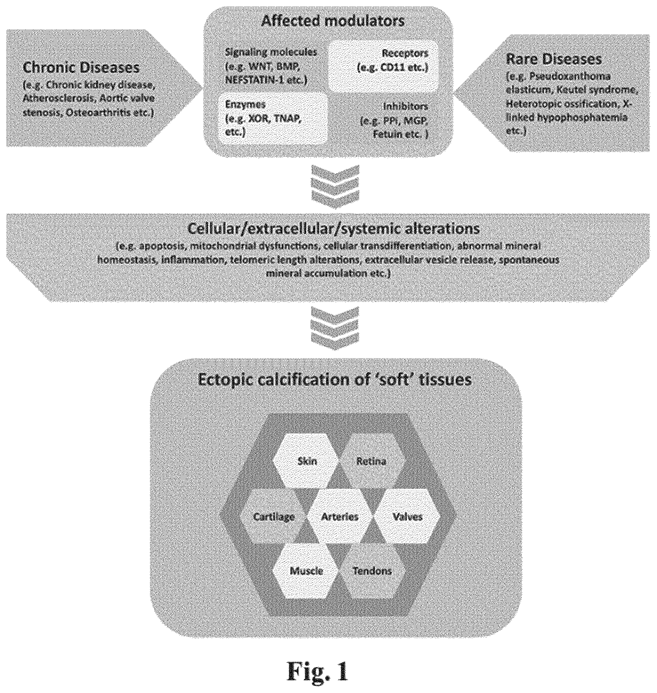

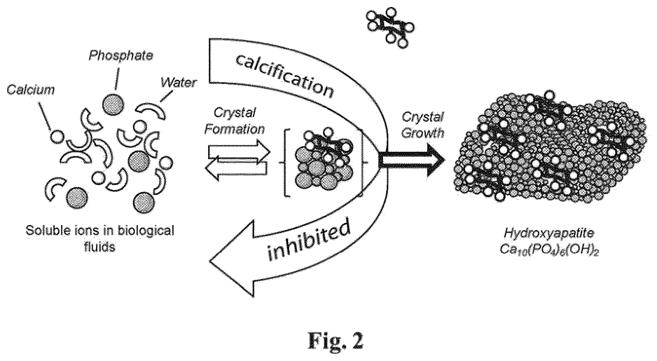

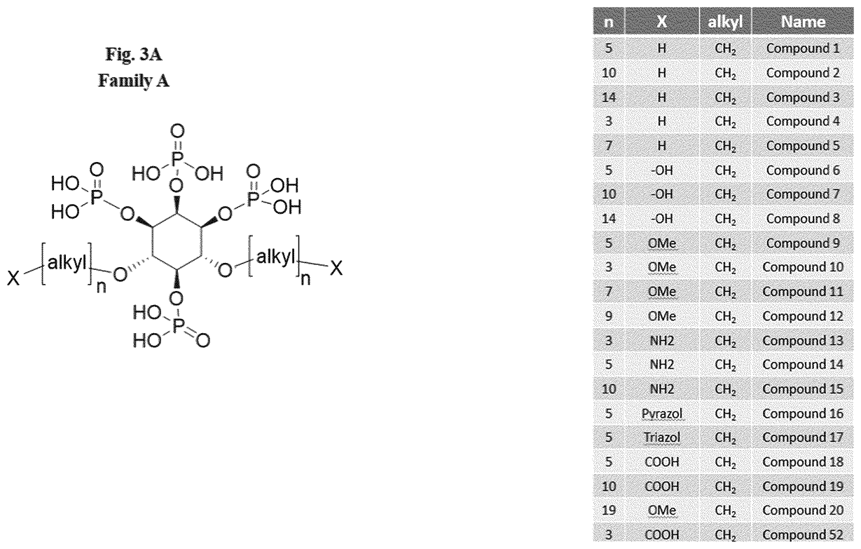

### DETAILED DESCRIPTION OF THE INVENTION

The present invention refers to IP4-4,6 substituted derivative compounds of general formula I:

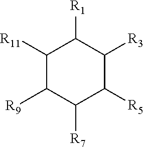

a pharmaceutically acceptable salt thereof, or a combination thereof, for use in the treatment, inhibition of progression, and prevention of ectopic calcification and the consequences thereof. In some aspects, n R₁, R₃, R₇, and R₁₁ are OPO₃²⁻ or OPSO₂²⁻, and R₅ and R₉ are identical. In some aspects, R₅ and R₉ are not identical. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt.

Also provided are methods, pharmaceutical compositions and formulations, methods of use, articles of manufacture, and kits for the treatment, inhibition of progression, and treatment of ectopic calcification and the consequences thereof and disease and/or conditions related to ectopic calcification and the consequences thereof.

In order that the present invention can be more readily understood, certain terms are first defined below. As used in this application, except as otherwise expressly provided herein, each of the following terms shall have the meaning set forth below. Additional definitions are set forth throughout the application.

### I. Definitions

The invention includes aspects in which exactly one member of the group is present in, employed in, or otherwise relevant to a given product or process. The invention includes aspects in which more than one, or all of the group members are present in, employed in, or otherwise relevant to a given product or process.

Unless defined otherwise, all technical and scientific terms used herein have the same meaning as commonly understood by one of ordinary skill in the art to which this invention is related. For example, the Concise Dictionary of Biomedicine and Molecular Biology, Juo, Pei-Show, 2^{nd} Ed., 2002, CRC Press; The Dictionary of Cell and Molecular Biology, 3^{rd} Ed., 1999, Academic Press; and the Oxford Dictionary of Biochemistry and Molecular Biology, Revised, 2000, Oxford University Press, provide one of skill with a general dictionary of many of the terms used in this invention.

Units, prefixes, and symbols are denoted in their Système International d'Unités (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention.

Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

About: The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value.

When particular values or compositions are provided in the application and claims, unless otherwise stated, the meaning of “about” should be assumed to be within an acceptable error range for that particular value or composition. When the term “about” is used in conjunction with a numerical range, it modifies that range by extending the boundaries above and below the numerical values set forth. Thus, “about 10-20” means “about 10 to about 20.” In general, the term “about” can modify a numerical value above and below the stated value by a variance of, e.g., 10 percent, up or down (higher or lower).

And/or: “And/or” where used herein is to be taken as specific invention of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Angina pectoris: As used herein, the terms “angina pectoris” and “chronic stable angina” relate to chest pain or discomfort that occurs as the result of myocardial ischemia. It is a common presenting symptom among patients with coronary artery disease (CAD). Approximately 9.8 million Americans are estimated to experience angina annually, with 500,000 new cases of angina occurring every year.

Approximately: As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1% or less in either direction (greater than or less than) of the stated reference value unless otherwise stated or otherwise evident from the context (except where such number would exceed 100% of a possible value).

Bolus administration: As used herein, the terms “bolus administration” and “bolus injection” refer a fast intravenous injection lasting less than 10 seconds, or an intravenous infusion lasting less than 3 minutes.

Calcific aortic valve stenosis: As used herein, the terms “calcific aortic valve stenosis” and “CAVS” refer to one of the most prevalent heart valve disorders in developed countries characterized by progressive fibro-calcific remodeling and thickening of the aortic valve leaflets. Over the years, this disease evolves to cause severe obstruction to cardiac outflow. In developed countries, CAVS is the third-most frequent cardiovascular disease after coronary artery disease and systemic arterial hypertension, with a prevalence of 0.4% in the general population and 1.7% in the population >65 years old (Lindman B, et al., Nat Rev Dis Primers. 2016; 2:16006).

Calciphylaxis: As used herein, the terms “calciphylaxis” and “CUA” refer to is a serious, uncommon disease in which calcium accumulates in small blood vessels of the fat and skin tissues. CUA causes blood clots, painful skin ulcers and may cause serious infections that can lead to death. CUA subjects usually suffer kidney failure and may require dialysis. The condition can also occur in people without kidney disease.

Cardiovascular disease in chronic kidney disease: As used herein, the terms “cardiovascular disease in CKD patients” refer to the higher risk of CKD patients of developing cardiovascular diseases such as e.g., coronary artery disease, heart failure, arrhythmias, and sudden cardiac death. Although the incidence and prevalence of cardiovascular events is already significantly higher in patients with early CKD stages (CKD stages 1-3) compared with the general population, patients with advanced CKD stages (CKD stages 4-5) exhibit a markedly elevated risk (Jankowski J, et al., Circulation 2021; 143 (11): 1157-1172).

Cardiovascular disease linked to aging: As used herein, the term “cardiovascular disease linked to aging” refers to the fact that adults over 65 years of age have a high risk of cardiovascular diseases such as heart failure, coronary artery diseases, hypertension, cerebrovascular disease, peripheral arterial disease, valvular disease, and cardiac arrhythmias. Aging is associated with changes in the heart and blood vessels that increase a person's risk of developing cardiovascular disease (North B, et al., Circ Res. 2012; 110 (8): 1097-1108). Age is an established independent risk factor for cardiovascular disease.

Comprising: It is understood that wherever aspects are described herein with the language “comprising,” otherwise analogous aspects described in terms of “consisting of” and/or “consisting essentially of” are also provided.

Compound: As used herein, the term “compound,” is meant to include any and all free bases, isomers, and isotopes of the structure depicted. As used herein, the term “isomer” means any geometric isomer, tautomer, zwitterion, stereoisomer, enantiomer, or diastereomer of a compound. Compounds can include one or more chiral centers and/or double bonds and can thus exist as stereoisomers, such as double-bond isomers (i.e., geometric E/Z isomers) or diastereomers (e.g., enantiomers (i.e., (+) or (−)) or cis/trans isomers). The present invention encompasses any and all isomers of the compounds described herein, including stereomerically pure forms (e.g., geometrically pure, enantiomerically pure, or diastereomerically pure) and enantiomeric and stereoisomeric mixtures (e.g., racemates). Enantiomeric and stereometric mixtures of compounds and means of resolving them into their component enantiomers or stereoisomers are well-known. A compound, salt, or complex of the present invention can be prepared in combination with solvent or water molecules to form solvates and hydrates by routine methods. In some aspects, the term compound is used to refer to an IP4-4,6 substituted derivative of the present invention.

Consequences thereof: The phrase “consequences thereof” when referring to a disease or condition disclosed herein refers to symptoms, sequelae, complications, and combinations thereof associated with the disease or condition. As used herein, the term “symptom” refers to subjective or physical sign, indication, or evidence of disease or physical disturbance observed by the subject. In general, the term refers to any morbid phenomenon or departure from the normal in structure, function, or sensation, experienced by a subject and indicative of disease. Symptoms are felt or noticed by the individual experiencing the symptom but may not easily be noticed by others. In some aspects, a symptom can be a mild symptom, a moderate symptom, or severe symptom. As used herein, the term “mild symptom” refers to a symptom that is not life threatening and does not require hospitalization or intensive care treatment (e.g., at a hospital ICU). As used herein, the term “moderate symptom” refers to a symptom that may become life threatening and may require hospitalization. As used herein, the term “severe symptom” refers to a symptom that is life threatening and requires intensive care treatment (e.g., at a hospital ICU). As used herein, the term “complication” refers to a pathological process or event occurring during a disease or condition that is not an essential part of the disease or condition; where it may result from the disease/condition or from independent causes. Accordingly, the term complication refers to medical/clinical problems that are observed in subjects diagnosed with a disease or conditions disclosed herein. In some aspects, a complication can be temporary. In some aspects, a complication can be chronic or permanent. As used herein, the term “sequela” refers to a long term, chronic, or permanent complication.

Critical limb ischemia: As used herein, the terms “critical limb ischemia” and “CLI” refer to a severe obstruction of the arteries which markedly reduces blood flow to the extremities and progresses to the point of severe pain and even skin ulcers, sores, or gangrene. Critical limb ischemia is a very severe condition of peripheral artery disease.

Ectopic calcification: As used herein, the term “ectopic calcification” refers to an inappropriate biomineralization occurring in soft tissues. Ectopic calcifications are typically composed of calcium phosphate salts, including hydroxyapatite, but can also consist of calcium oxalates and octacalcium phosphate as seen in kidney stones.

Effective amount: As used herein, the term “effective amount” of a therapeutic agent, in reference to (i) an IP4-4,6 substituted derivative of the present invention, (ii) any dosage form, pharmaceutical composition, or formulation disclosed herein comprising at least one IP4-4,6 substituted derivative of the present invention, or (iii) a combination of an IP4-4,6 substituted derivative of the present invention with one or more additional therapeutic agents), is that amount sufficient to effect beneficial or desired results. In some aspects, the beneficial or desired results are, for example, clinical results, and, as such, an “effective amount” depends upon the context in which it is being applied. The term “effective amount” can be used interchangeably with “effective dose,” “therapeutically effective amount,” or “therapeutically effective dose.”

The term effective amount relates to the specific use of an IP4-4,6 substituted derivative. For example, when an IP4-4,6 substituted derivative is used to inhibit the formation or growth of a calcium salt/crystal (e.g., a calcium phosphate, HAP), an effective amount would be an amount of the IP4-4,6 substituted derivative capable of achieving the desired effect (e.g., the reduction of HAP crystallization/formation in blood serum or plasma).

Enteral administration: As used herein, the term “enteral administration” and the related term “enterally” refer to any administration of an IP4-4,6 substituted derivative of the present invention or a pharmaceutical composition comprising said derivative via the gastrointestinal tract. Enteral administration includes, but is not limited to, the oral, sublingual, and rectal routes of administration.

General arterial calcification of infancy: As used herein, the terms “general arterial calcification of infancy” and “GACI” relates to a disorder affecting the circulatory system that becomes apparent before birth or within the first few months of life, and which is characterized by abnormal calcification of the arteries and thickening of the arterial walls. These changes lead to stenosis and stiffness of the arteries, resulting in heart failure in some affected individuals, with signs and symptoms including difficulty breathing, edema, cyanosis, hypertension, and cardiomegaly.

IP4-4,6 substituted derivatives of the present invention: As used herein, the term “IP4-4,6 substituted derivative of the present invention” and grammatical variants thereof refers to a compound of formula I:

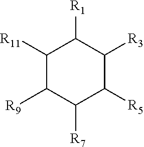

wherein R₁, R₃, R₇, and R₁₁ are OPO₃²⁻ or OPSO₂²⁻, and R₅ and R₉ may or may not be identical, and wherein the R₅ and R₉ substituents are those disclosed in compound Families A, B, C, and D described in detail below, and salts thereof (e.g., pharmaceutically acceptable salts thereof). In some aspects, the term IP4-4,6 substituted derivative of the present invention encompasses Compounds 1 to 53, any salt thereof (e.g., a sodium salt), and any combination thereof. In some aspects, the term IP4-4,6 substituted derivative of the present invention encompasses a compound of formula I which is an intermediate in the synthesis of Compounds 1 to 53, e.g., a compound selected from the group consisting of the compounds listed in Table 1, any salt thereof (e.g., a sodium salt), and any combination thereof. In some aspects, the term IP4-4,6 substituted derivative of the present invention encompasses a compound of formula I which is Compound 1 to Compound 53 and a compound selected from the group consisting of the compounds listed in Table 1, any salt thereof (e.g., a sodium salt), and any combination thereof.

Group consisting of Compound 1 to Compound 53: In the context of the present invention, references to a “group consisting of Compound 1 to Compound 53”, refers to a group of compounds that comprises Compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, and 53. In some aspects, the group consisting of Compound 1 to Compound 53 also comprises combinations thereof. In some aspects, a combination of compounds from the group consisting of Compound 1 to Compound 53 can comprise 2, 3, 4, 5, 6, 7, 8, 9, 10 or more compounds from the group consisting of Compound 1 to Compound 53.

Group consisting of the compounds listed in Table 1: In the context of the present invention, references to “Group consisting of the compounds listed in Table 1”, refers to a group of intermediate compounds used, e.g., for the synthesis of an IP4-4,6 substituted derivative of the present invention (e.g, a compound selected from the group consisting of Compound 1 to Compound 53) i.e., Intermediates II-1 to II-27, Intermediates III-1 to III-47, Intermediates IV-1 to IV-48, Intermediates VI′-1 to IV′-2, Intermediate V-1, Intermediate VI-1, Intermediates VII-1 to VII-2, Intermediates VIII-1 to VIII-5, Intermediates IX-1 to IX-5, Intermediates X-1 to X-3, Intermediates XI-1 to XI-2, Intermediates XII-1 to XII-2 and Intermediates XIII-1 to XIII-3. In some aspects, the group consisting of the compounds listed in Table 1 also comprises combinations thereof. In some aspects, a combination of compounds from the group consisting of the compounds listed in Table 1 can comprise 2, 3, 4, 5, 6, 7, 8, 9, 10 or more compounds from the group consisting of the compounds listed in Table 1.

Kidney failure-related diseases: As used herein, the term “kidney failure-related diseases” refers to disease processes of a widely diverse nature in individuals with kidney damage and can refer, but is not limited, to any disease related to calcium or calcium metabolism disorders, such as adynamic bone, bone cancer, bone mineral disease, calcific tendinitis, calcinosis cutis, calciphylaxis, renal lithiasis, cardiovascular calcification, cardiovascular disease, osteomalacia, osteoporosis, podagra, and rheumatoid arthritis. Other kidney failure-related diseases may be of the cardiovascular type including, but not limited to, aneurysm, angina pectoris, arteriosclerosis, atherosclerosis, cardiac disease, cardiovascular disease linked to aging, cerebrovascular disease, coronary disease, heart failure, hypertension, myocardial infarction, peripheral vascular disease, and thrombosis.

The patient with renal impairment may suffer concomitant cardiovascular accidents, events or diseases (e.g., ischemia, arrhythmia, myocardial infarction, stroke).

Non-bolus administration: As used herein, the terms “non-bolus type” and “non-bolus administration” refer to an intravenous injection lasting 10 or more seconds, or an intravenous infusion lasting 3 or more minutes.

Parenteral administration: As used herein, the term “parenteral administration” and the related term “parenterally” refer to the administration of an IP4-4,6 substituted derivative of the present invention characterized by the physical breaching of a tissue of a subject and the administration of the derivative through said breach in the tissue. Parenteral administration includes, but is not limited to, the administration of an IP4-4,6 substituted derivative of the present invention (e.g., a compound selected from the group consisting of Compound 1 to Compound 53) or a pharmaceutical composition comprising the derivative, by the application of the derivative or the composition through, for instance, a surgical incision or through a tissue-penetrating non-surgical wound. In particular, parenteral administration includes, but is not limited to, the epidural, intraarterial, intradermal, intrathecal, intramuscular, intraperitoneal, intrasternal injection, intravascular, intravenous, intravenous infusion, spinal, subcutaneous, and subcutaneous depot routes of administration.

Peripheral arterial disease: As used herein, the terms “peripheral arterial disease” and “PAD” refer to a narrowing and/or stiffening of the peripheral arteries to the legs (most commonly), stomach, arms, and head. Symptoms include intermittent claudication (leg pain when walking which resolves with rest), skin ulcers, bluish skin, cold skin, or poor nail and hair growth.

Prevention: As used herein, the terms “prevent”, “preventing”, and “prevention” refer to inhibiting the inception or decreasing the occurrence of a disease or condition in a subject (e.g., avert ectopic calcification or the consequence thereof in a subject.

Primary hyperoxaluria: As used herein, the terms “primary hyperoxaluria” or “PH” refer to a disorder of glyoxylate metabolism characterized by an excess of oxalate resulting in kidney stones, nephrocalcinosis and ultimately renal failure and systemic oxalosis. There are 3 types of PH. Primary hyperoxaluria type 1 (PH1) is caused by a deficiency of the liver peroxisomal enzyme alanine: glyoxylate-aminotransferase (AGT), which catalyzes the conversion of glyoxylate to glycine. When AGT activity is absent, glyoxylate is converted to oxalate, which forms insoluble calcium oxalate crystals that accumulate in the kidney and other organs. Primary hyperoxaluria type 2 (PH2), caused by mutations in the GRHPR gene, is an inherited disease in which the lack of a particular liver enzyme causes the body to accumulate excess amounts of oxalate. Primary hyperoxaluria type 3 (PH3) is characterized by recurring calcium oxalate stones beginning in childhood or adolescence and, on occasion, nephrocalcinosis or reduced kidney function.

Pseudogout: As used herein, the terms “pseudogout”, also known as “calcium pyrophosphate dihydrate (CPPD) crystal deposition disease” or “pyrophosphate arthropathy” refer to a rheumatologic disorder believed to be caused by calcium pyrophosphate crystal accumulation in connective tissues, particularly joints such as the knee joint.

Pseudoxanthoma elasticum: As used herein, the terms “Pseudoxanthoma elasticum” and “PXE” refer to a genetic metabolic disease with autosomal recessive inheritance caused by mutations in the ABCC6 gene. The lack of functional ABCC6 protein leads to ectopic calcification that is most apparent in the elastic tissues of the skin, eyes, and blood vessels. The clinical prevalence of PXE has been estimated at between 1 per 100,000 and 1 per 25,000, with slight female predominance.

Ranges: As described herein, any concentration range, percentage range, ratio range or integer range is to be understood to include the value of any integer within the recited range and, when appropriate, fractions thereof (such as one tenth and one hundredth of an integer), unless otherwise indicated.

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain aspects, the mammal is a human subject. In other aspects, a subject is a human patient. In a particular aspect, a subject is a human patient with a pathological crystallization or at risk of having pathological crystallizations.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the term “therapeutic agent” is used in a broad sense to include a composition comprising an IP4-4,6 substituted derivative of the present invention that can provide a significant therapeutic benefit to a subject in need thereof. In some aspects, the subject in need thereof is a subject suffering or at risk of developing a disease or condition associated to pathological crystallization (e.g., a calcium phosphate or HAP crystallization). Thus, in general, a therapeutic agent according to the present invention can be an IP4-4,6 substituted derivative of the present invention, alone or in combination with one or more additional therapeutic agents, that is administered in an amount sufficient to effect beneficial or desired results.

The term therapeutic agent also encompasses prophylactic, diagnostic or imaging agents comprising an IP4-4,6 substituted derivative of the present invention, wherein the therapeutic agent is administered (i.e., topically, enterally or parenterally). Therapeutic agents of the present invention include agents that inhibit the formation or growth of calcium salts/crystals (e.g., calcium phosphates, HAP) and/or can ameliorate and/or prevent any symptom associated with pathological crystallization.

Topical administration: As used herein, the term “topical administration” and the related term “topically” refer to any administration of an IP4-4,6 substituted derivative of the present invention or a pharmaceutical composition comprising said derivative by applying the derivative or composition to a particular place on or in the body, such as the skin or a mucous membrane. Topical administration includes, but is not limited to, the aural, cutaneous, nasal, transdermal, urethral, vaginal, and urethral routes of administration.

Treatment: As used herein, the terms “treat”, “treating”, and “treatment” refer to the administration of compound or pharmaceutical composition of the present invention for (i) slowing, (ii) inhibiting the progression, (iii) stopping, or (iv) reverting the progression of a disease or condition after its clinical signs have appeared. Control of the disease progression is understood to mean the beneficial or desired clinical results that include, but are not limited to, reduction of the symptoms, reduction of the duration of the disease, stabilization of pathological states (specifically to avoid additional deterioration), delaying the progression of the disease, improving the pathological state and remission (both partial and total). The control of progression of the disease also involves an extension of survival compared with the expected survival if treatment was not applied. Within the context of the present invention, the terms “treat” and “treatment” refer specifically to (a) stopping, reducing, inhibiting the progression or reverting the development of ectopic calcifications and the consequences therefor, (b) improving the mobility or walking ability (e.g., velocity, distance, endurance) in a subject administered with the compounds or pharmaceutical compositions of the present invention (c) reducing or improving the quality of life in a subject administered with the compounds or pharmaceutical compositions of the present invention.

ug, uM, uL: As used herein, the terms “ug,” “uM,” and “uL” are used interchangeably with “μg,” “μM,” and “μL” respectively.

### II. IP4-4,6 Substituted Derivatives

The present invention discloses IP4-4,6 substituted derivatives, their methods of synthesis and their uses. In some aspects, the IP4-4,6 substituted derivative is a compound of general formula I:

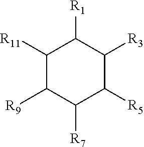

wherein R₁, R₃, R₇, and R₁₁ independently represent OPO₃²⁻ or OPSO₂²⁻, and R₅ and R₉ are substituent groups. In some aspects, R₅ and R₉ are identical substituent groups. In some aspects, R₅ and R₉ are not identical substituent groups. In some aspects, the IP4-4,6 substituted derivative of the present invention is an acceptable salt (e.g., a pharmaceutically acceptable salt) or combination thereof of a compound of formula I.

In some aspects, not all negative charges in an IP4-4,6 derivative of the present invention are compensated by charges in positively charged ions. Accordingly, an IP4-4,6 derivative of the present invention can be, for example, a tetraionic salt (e.g., tetrasodium salt), a pentaionic salt (e.g., pentasodium salt), a hexaionic salt (e.g., hexasodium salt), a heptaionic salt (e.g., heptasodium salt), an octaionic salt (e.g., octasodium salt), a nonaionic salt (e.g., nonasodium salt) or a decaionic salt (e.g., decasodium salt). In some aspects, the presence of additional negatively charges group in a IP4-4,6 substituted derivatives can lead to the formation of complexes with additional ions. In some aspects, the IP4-4,6 derivative of the present invention is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. Formula I and the rest of formulas presented in the invention are meant to encompass any isomers of the compounds covered thereby.

The term “alkenyl” or “alkenyl chain” in the context of the present invention refers to a linear or branched alkyl chain (e.g., containing between 2 and 10 carbon atoms) containing one or more double bonds, either substituted or non-substituted. Examples include, amongst others, ethenyl, 1-propenyl, 2-propenyl, isopropenyl, 1-butenyl, 2-butenyl, 3-butenyl, and 1,3-butadienyl.

The term “alkyl” or “alkyl chain” in the context of the present invention refers to a saturated hydrocarbon moiety, which can be linear, branched, cyclic or cyclic with linear or branched side chains. The term alkyl includes partially unsaturated hydrocarbons such as propenyl. Examples are methyl, ethyl, propyl, isopropyl, n- or isobutyl, n- or cyclohexyl. The term alkyl can extend to alkyl groups linked or bridged by hetero atoms. Hetero atoms in the context of the present invention are nitrogen (N), sulfur(S), oxygen (O), and halogen.

The term “alkynyl” or “alkynyl chain” in the context of the present invention refers to a linear or branched alkyl chain (e.g., containing between 2 and 10 carbon atoms) containing one or more triple bonds, either substituted or non-substituted. Examples include, amongst others, ethynyl, propynyl, 1-butynyl, and 3-butynyl.

An “amine function” or “amine group” is a function NRR′, with R and R′ selected independently, e.g., from hydrogen (—H) and an alkyl group such as an C₁-Cₙ alkyl, wherein n is and integer between 0 and 20.

A “hydroxy function” or “hydroxy group” is OH.

A “carboxylic acid function” or “carboxylic acid group” is COOH or its anion, COO⁻.

A “carboxylic amide” is CONRR′ or NCOR, with R and R′ selected independently, e.g., from hydrogen (—H) and an alkyl group such as an C₁-Cₙ alkyl, wherein n is and integer between 0 and 20.

A “carbocycle” refers to a three- to 10-membered carbocyclic ring that can be saturated, partially unsaturated or aromatic (e.g., phenol, cyclopentane, cyclopropane) and which is bound to the rest of the molecule via any available C atom. The term includes carbocyclic rings substituted with one or more heteroatoms (e.g., N, O, halogen atom).

A “heterocycle” refers to a three- to 10-membered cyclic ring containing at least one heteroatom selected from amongst N, O, and S, that can be saturated, partially unsaturated or aromatic (e.g., triazole, piperazine, pyrazole, thiophene) and which is bound to the rest of the molecule via any available C atom or N atom. The term includes heterocycle rings substituted with one or more halogen atoms.

A “Cy” refers to a cyclic linker comprising a carbocycle or a heterocycle. Examples of carbocycles and heterocycles include, amongst others, 1,3-phenyl, 1,4-phenyl, naphthyl, thienyl, furyl, pyrrolyl, thiazolyl, isothiazolyl, imidazolyl, pyrazolyl, 1,2,3-triazolyl, 1,2,4-triazolyl, tetrazolyl, 1,3,4-thiadiazolyl, 1,2,4-thiadiazolyl, pyridyl, piperazyl, pyrazinyl, pyrimidinyl, pyridazinyl, benzimidazolyl, benzofuranyl, isobenzofuranyl, indolyl, isoindolyl, benzothiophenyl, benzothiazolyl, cyclopropyl, cyclobutyl, cyclopentyl, cyclohexyl, cycloheptyl, azetidinyl, and aziridinyl.

A “halogen” group refers to fluorine, chlorine, bromine or iodine.

The term “OPO₃²⁻” in the context of the present invention refers also indistinctly to OPO₃H⁻ and OPO₃H₂.

In some aspects, the IP4-4,6 substituted derivatives of the present invention or intermediate compounds disclosed herein can be detected and/or quantified using the methods disclosed in U.S. Pat. No. 9,612,250. See also, U.S. Pat. Nos. 8,377,909, 8,778,912 and US20070066574.

The IP4-4,6 substituted derivatives of the present invention can be present in any form commonly used in pharmaceutical technology. Particular aspects include, but are not limited to, the sodium salt, magnesium salt, potassium salt, ammonium salt, free acid, or a mixture of the preceding forms. Other pharmaceutically acceptable salts are known to the skilled artisan and can be obtained by methods previously described (Haynes M, et al., J. Pharmaceutical Sci. 2005; 94:2111-2120).

In some aspects, R₁, R₃, R₇, and R₁₁ independently represent phosphate (—OPO₃²⁻) and R₅/R₉ corresponds to the formula —O-(alkyl)ₙ-X (i.e., R₅/R₉ is an alkyl chain with or without ramification), wherein n is an integer between 1 and 20, wherein the terminal group X is selected from the group consisting of —H, —OR, —NRR′, —COOR, —CONRR′, —NHCOR, —NHCOOR, —OCONR, —NHSO₂R, —NHCONRR′, halogen, —CF₃, alkyl, alkenyl, alkynyl, carbocycle (saturated or unsaturated), and heterocycle (saturated or unsaturated), and wherein R and R′ are H or an alkyl group. Throughout the present invention, this family of IP4-4,6 substituted derivatives is designated Family A.

In some aspects, R₁, R₃, R₇, and R₁₁ independently represent phosphate (—OPO₃²⁻) and R₅/R₉ corresponds to the formula —O-(alkyl)_{y}-Cy-(alkyl)_{y′}-Z (i.e., R₅/R₉ comprises two alkyl chains connected by a cyclic linker interposed between the two alkyl chains), wherein each alkyl comprises y carbon (e.g., CH₂) units, wherein y and y′ are an integer between 0 and 10, wherein the distal alkyl chain comprises a terminal group Z selected from the group consisting of alkyl, —COR, —OR, —NRR′, —COOR, —CONRR′, —NHCOR, —NHCOOR, —OCONR—NHSO₂R, —NHCONRR′, halogen, and —CF₃, and wherein R and R′ are H or an alkyl group. Throughout the present invention, this family of IP4-4,6 substituted derivatives is designated Family B.

In some aspects, R₁, R₃, R₇, and R₁₁ independently represent phosphate (—OPO₃²⁻) and R₅/R₉ corresponds to the formula —O-(alkyl)_{y}-A-(alkyl)_{y′}-Z′ (i.e., two alkyl chains connected by a linker A interposed between the two alkyl chains), wherein each alkyl chain comprises y carbon (e.g., CH₂) units, wherein y and y′ are an integer between 0 and 10, wherein the linker A is selected from the group consisting of —CONR—, —NHCOO—, —NHSO₂—, —NHCONR—, —NHCO— and —OCONR—, wherein the distal alkyl chain comprises a terminal group Z′ selected from the group consisting of —OR, —NRR′, —COOR, —CONRR′, —NHCOR, —NHCOOR, —OCONR, —NHSO₂R, —NHCONRR′, carbocycle (saturated or unsaturated), heterocycle (saturated or unsaturated), and wherein R and R′ are H or an alkyl group. Throughout the present invention, this family of IP4-4,6 substituted derivatives is designated Family C.

In some aspects, the IP4-4,6 substituted derivative of Family D is an analog of a compound from Family A, B or C in which at least one of R₁, R₃, R₇ or R₁ is thiophosphate (—OPSO₂²⁻). Throughout the present invention, this family of IP4-4,6 substituted derivatives is designated Family D.

In some aspects of the IP4-4,6 substituted derivatives of Family D, one of R₁, R₃, R₇ or R₁₁ in a compound from Family A, B or C is thiophosphate (—OPSO₂²⁻). In some aspects of the IP4-4,6 substituted derivatives of Family D, two of R₁, R₃, R₇ or R₁₁ in a compound from Family A, B or C are thiophosphate (—OPSO₂²⁻). In some aspects of the IP4-4,6 substituted derivatives of Family D, three of R₁, R₃, R₇ or Rui in a compound from Family A, B or C are thiophosphate (—OPSO₂²⁻). In some aspects of the IP4-4,6 substituted derivatives of Family D, R₁, R₃, R₇ or R₁ in a compound from Family A, B or C are thiophosphate (—OPSO₂²⁻).

In some aspects of the IP4-4,6 substituted derivatives of Family D, the Family D compound contains thiophosphate (—OPSO₂²⁻) only at position R₁. In some aspects of the IP4-4,6 substituted derivatives of Family D, the Family D compound contains thiophosphate (—OPSO₂²⁻) only at position R₃. In some aspects of the IP4-4,6 substituted derivatives of Family D, the Family D compound contains thiophosphate (—OPSO₂²⁻) only at position R₇. In some aspects of the IP4-4,6 substituted derivatives of Family D, the Family D compound contains thiophosphate (—OPSO₂²⁻) only at position R₁. In some aspects of the IP4-4,6 substituted derivatives of Family D, the Family D compound contains thiophosphate (—OPSO₂²⁻) at positions R₁, R₃, R₇, and R₁₁. In some aspects of the IP4-4,6 substituted derivatives of Family D, the Family D compound contains thiophosphate (—OPSO₂²⁻) at positions R₁ and R₃. In some aspects of the IP4-4,6 substituted derivatives of Family D, the Family D compound contains thiophosphate (—OPSO₂²⁻) at positions R₁ and R₇. In some aspects of the IP4-4,6 substituted derivatives of Family D, the Family D compound contains thiophosphate (—OPSO₂²⁻) at positions R₁ and R₁₁. In some aspects of the IP4-4,6 substituted derivatives of Family D, the Family D compound contains thiophosphate (—OPSO₂²⁻) at positions R₃ and R₇. In some aspects of the IP4-4,6 substituted derivatives of Family D, the Family D compound contains thiophosphate (—OPSO₂²⁻) at positions R₃ and R₁₁. In some aspects of the IP4-4,6 substituted derivatives of Family D, the Family D compound contains thiophosphate (—OPSO₂²⁻) at positions R₇ and R₁₁. In some aspects of the IP4-4,6 substituted derivatives of Family D, the Family D compound contains thiophosphate (—OPSO₂²⁻) at positions R₁, R₃, and R₇. In some aspects of the IP4-4,6 substituted derivatives of Family D, the Family D compound contains thiophosphate (—OPSO₂²⁻) at positions R₁, R₃, and R₁₁. In some aspects of the IP4-4,6 substituted derivatives of Family D, the Family D compound contains thiophosphate (—OPSO₂²⁻) at positions R₃, R₇, and R₁₁. In some aspects of the IP4-4,6 substituted derivatives of Family D, the Family D compound contains thiophosphate (—OPSO₂²⁻) at positions R₁, R₇, and R₁₁.

In some aspects, an IP4-4,6 substituted derivative of the present invention comprises an IP4-4,6 substituted derivative from Family A, Family B, Family C, Family D or a combination thereof.

In some aspects, an IP4-4,6 substituted derivative of the present invention comprises an IP4-4,6 substituted derivative from Family A. In some aspects, an IP4-4,6 substituted derivative of the present invention comprises an IP4-4,6 substituted derivative from Family B. In some, an IP4-4,6 substituted derivative of the present invention comprises an IP4-4,6 substituted derivative from Family C. In some aspects, an IP4-4,6 substituted derivative of the present invention comprises an IP4-4,6 substituted derivative from Family D.

In some aspects, an IP4-4,6 substituted derivative of the present invention comprises or consists of an IP4-4,6 substituted derivative selected from the group consisting of Compounds 1 to 53 and combinations thereof. In some aspects, an IP4-4,6 substituted derivative of the present invention comprises or consists of an IP4-4,6 substituted derivative selected from the group consisting of compounds 1 to 30 and combinations thereof. In some aspects, an IP4-4,6 substituted derivative of the present invention comprises or consists of an IP4-4,6 substituted derivative selected from the group consisting of compounds 31 to 44 and combinations thereof. In some aspects, an IP4-4,6 substituted derivative of the present invention comprises or consists of an IP4-4,6 substituted derivative selected from the group consisting of compounds 45 to 49 and combinations thereof. In some aspects, an IP4-4,6 substituted derivative of the present invention comprises or consists of an IP4-4,6 substituted derivative selected from the group consisting of compounds 50 to 51 and combinations thereof. In some aspects, an IP4-4,6 substituted derivative of the present invention comprises or consists of Compound 1. In some aspects, an IP4-4,6 substituted derivative of the present invention comprises or consists of Compound 2. In some aspects, an IP4-4,6 substituted derivative of the present invention comprises or consists of Compound 3. In some aspects, an IP4-4,6 substituted derivative of the present invention comprises or consists of Compound 4. In some aspects, an IP4-4,6 substituted derivative of the present invention comprises or consists of Compound 5. In some aspects, an IP4-4,6 substituted derivative of the present invention comprises or consists of Compound 6. In some aspects, an IP4-4,6 substituted derivative of the present invention comprises or consists of Compound 7. In some aspects, an IP4-4,6 substituted derivative of the present invention comprises or consists of Compound 8. In some aspects, an IP4-4,6 substituted derivative of the present invention comprises or consists of Compound 9. In some aspects, an IP4-4,6 substituted derivative of the present invention comprises or consists of Compound 10. In some aspects, an IP4-4,6 substituted derivative of the present invention comprises or consists of Compound 11. In some aspects, an IP4-4,6 substituted derivative of the present invention comprises or consists of Compound 12. In some aspects, an IP4-4,6 substituted derivative of the present invention comprises or consists of Compound 13. In some aspects, an IP4-4,6 substituted derivative of the present invention comprises or consists of Compound 14. In some aspects, an IP4-4,6 substituted derivative of the present invention comprises or consists of Compound 15. In some aspects, an IP4-4,6 substituted derivative of the present invention comprises or consists of Compound 16. In some aspects, an IP4-4,6 substituted derivative of the present invention comprises or consists of Compound 17. In some aspects, an IP4-4,6 substituted derivative of the present invention comprises or consists of Compound 18. In some aspects, an IP4-4,6 substituted derivative of the present invention comprises or consists of Compound 19. In some aspects, an IP4-4,6 substituted derivative of the present invention comprises or consists of Compound 20. In some aspects, an IP4-4,6 substituted derivative of the present invention comprises or consists of Compound 21. In some aspects, an IP4-4,6 substituted derivative of the present invention comprises or consists of Compound 22. In some aspects, an IP4-4,6 substituted derivative of the present invention comprises or consists of Compound 23. In some aspects, an IP4-4,6 substituted derivative of the present invention comprises or consists of Compound 24. In some aspects, an IP4-4,6 substituted derivative of the present invention comprises or consists of Compound 25. In some aspects, an IP4-4,6 substituted derivative of the present invention comprises or consists of Compound 26. In some aspects, an IP4-4,6 substituted derivative of the present invention comprises or consists of Compound 27. In some aspects, an IP4-4,6 substituted derivative of the present invention comprises or consists of Compound 28. In some aspects, an IP4-4,6 substituted derivative of the present invention comprises or consists of Compound 29. In some aspects, an IP4-4,6 substituted derivative of the present invention comprises or consists of Compound 30. In some aspects, an IP4-4,6 substituted derivative of the present invention comprises or consists of Compound 31. In some aspects, an IP4-4,6 substituted derivative of the present invention comprises or consists of Compound 32. In some aspects, an IP4-4,6 substituted derivative of the present invention comprises or consists of Compound 33. In some aspects, an IP4-4,6 substituted derivative of the present invention comprises or consists of Compound 34. In some aspects, an IP4-4,6 substituted derivative of the present invention comprises or consists of Compound 35. In some aspects, an IP4-4,6 substituted derivative of the present invention comprises or consists of Compound 36. In some aspects, an IP4-4,6 substituted derivative of the present invention comprises or consists of Compound 37. In some aspects, an IP4-4,6 substituted derivative of the present invention comprises or consists of Compound 38. In some aspects, an IP4-4,6 substituted derivative of the present invention comprises or consists of Compound 39. In some aspects, an IP4-4,6 substituted derivative of the present invention comprises or consists of Compound 40. In some aspects, an IP4-4,6 substituted derivative of the present invention comprises or consists of Compound 41. In some aspects, an IP4-4,6 substituted derivative of the present invention comprises or consists of Compound 42. In some aspects, an IP4-4,6 substituted derivative of the present invention comprises or consists of Compound 43. In some aspects, an IP4-4,6 substituted derivative of the present invention comprises or consists of Compound 44. In some aspects, an IP4-4,6 substituted derivative of the present invention comprises or consists of Compound 45. In some aspects, an IP4-4,6 substituted derivative of the present invention comprises or consists of Compound 46. In some aspects, an IP4-4,6 substituted derivative of the present invention comprises or consists of Compound 47. In some aspects, an IP4-4,6 substituted derivative of the present invention comprises or consists of Compound 48. In some aspects, an IP4-4,6 substituted derivative of the present invention comprises or consists of Compound 49. In some aspects, an IP4-4,6 substituted derivative of the present invention comprises or consists of Compound 50. In some aspects, an IP4-4,6 substituted derivative of the present invention comprises or consists of Compound 51. In some aspects, an IP4-4,6 substituted derivative of the present invention comprises or consists of Compound 52. In some aspects, an IP4-4,6 substituted derivative of the present invention comprises or consists of Compound 53.

In some aspects, an IP4-4,6 substituted derivative of the present invention comprises or consists of a Compound 1 analog, wherein R₁, R₃, R₇ or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 2 analog, wherein R₁, R₃, R₇ or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 3 analog, wherein R₁, R₃, R₇ or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 4 analog, wherein R₁, R₃, R₇ or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 5 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 6 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 7 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 8 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 9 analog, wherein R₁, R₃, R₇, or Rin; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 10 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 11 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 12 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 13 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 14 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 15 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 16 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 17 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 18 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 19 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 20 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 21 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 22 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 23 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 24 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 25 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 26 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 27 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 28 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 29 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 30 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 31 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 32 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 33 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 34 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 35 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 36 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 37 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 38 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 39 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 40 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 41 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 42 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 43 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 44 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 45 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 46 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 47 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 48 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 49 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 50 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 51 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 52 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁₁; R₃, R₇, and R₁₁; or R₁, R₇, and R₁₁ are thiophosphate (—OPSO₂²⁻). In some aspects, an IP4-4,6 substituted derivative of the present invention comprises or consists of a Compound 53 analog, wherein R₁, R₃, R₇, or R₁₁; R₁, R₃, R₇, and R₁₁; R₁ and R₃; R₁ and R₇; R₁ and R₁₁; R₃ and R₇; R₃ and R₁₁; R₇ and R₁₁; R₁, R₃, and R₇; R₁, R₃, and R₁; R₃, R₇, and R₁₁; or R₁, R₇, and R₁₁ are thiophosphate (—OPSO₂²⁻).

In some aspects, the alkyl moiety in the R₅ and R₉ substituent groups of the IP4-4,6 substituted derivative of the present invention includes at least one double or triple carbon bond, i.e., forming an alkenyl or alkynyl chain, respectively. In some aspects, the alkenyl chains in the R₅ and R₉ substituent groups are identical. In some aspects, the alkenyl chains in the R₅ and R₉ substituent groups are different. In some aspects, the alkynyl chains in the R₅ and R₉ substituent groups are identical. In some aspects, the alkynyl chains in the R₅ and R₉ substituent groups are different. In some aspects, the alkyl moiety in the R₅ and R₉ substituent groups includes at least one double carbon bond and at least one triple carbon bond, forming a combination of an alkenyl and an alkynyl chain. In some aspects, the combination of the alkenyl and the alkynyl chains in the R₅ and R₉ substituent groups is identical. In some aspects, the combination of the alkenyl and the alkynyl chains in the R₅ and R₉ substituent groups is different. In some aspects, the IP4-4,6 substituted derivative of the invention including at least one double or triple carbon bond in the alkyl moiety of the R₅ or R₉ substituent groups is a Family A compound. In some aspects, the Family A compound including at least one double or triple carbon bond is Compound 1 to Compound 30 and Compound 52.

In some aspects, an IP4-4,6 substituted derivative of the present invention comprises or consists of a compound of the following formula:

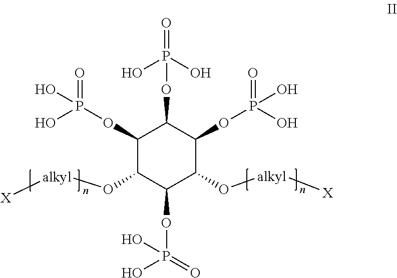

wherein n is an integer between 1 and 20, alkyl is CH₂ and X is —H. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, n is 5, alkyl is CH₂ and X is —H (Compound 1). In some aspects, n is 10, alkyl is CH₂ and X is —H (Compound 2). In some aspects, n is 14, alkyl is CH₂ and X is —H (Compound 3). In some aspects, n is 3, alkyl is CH₂ and X is —H (Compound 4). In some aspects, n is 7, alkyl is CH₂ and X is —H (Compound 5).

In some aspects, an IP4-4,6 substituted derivative of the present invention comprises or consists of a compound of the following formula:

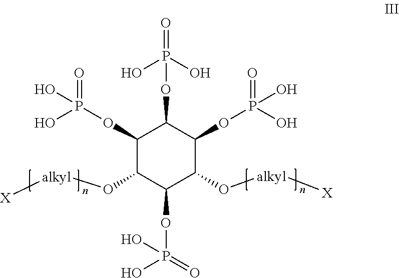

wherein n is an integer between 1 and 20, alkyl is CH₂ and X is selected from the group consisting of —OH and —OMe. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, n is 1, 2, 3, 4, 5, 6, 7, 8, 9, 10, 11, 12, 13, 14, 15, 16, 17, 18, 19 or 20. In some aspects, n is 5, alkyl is CH₂ and X is —OH (Compound 6). In some aspects, n is 10, alkyl is CH₂ and X is —OH (Compound 7). In some aspects, n is 14, alkyl is CH₂ and X is —OH (Compound 8). In some aspects, n is 5, alkyl is CH₂ and X is —OMe (Compound 9). In some aspects, n is 3, alkyl is CH₂ and X is —OMe (Compound 10). In some aspects, n is 7, alkyl is CH₂ and X is —OMe (Compound 11). In some aspects, n is 9, alkyl is CH₂ and X is —OMe (Compound 12). In some aspects, n is 19, alkyl is CH₂ and X is —OMe (Compound 20).

In some aspects, an IP4-4,6 substituted derivative of the present invention comprises or consists of a compound of the following formula:

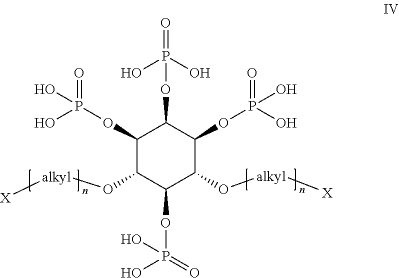

wherein n is an integer between 1 and 20, alkyl is CH₂ and X is an amine group. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is a hexasodium salt. In some aspects, n is 3, alkyl is CH₂ and X is —NH₂ (Compound 13). In some aspects, n is 5, alkyl is CH₂ and X is —NH₂ (Compound 14). In some aspects, n is 10 and X, alkyl is CH₂ is —NH₂ (Compound 15).

In some aspects, an IP4-4,6 substituted derivative of the present invention comprises or consists of a compound of the following formula:

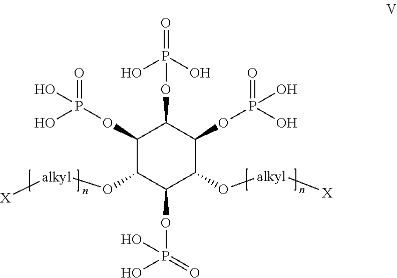

wherein n is an integer between 1 and 20, alkyl is CH₂ and X is pyrazole or triazole. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, n is 5, alkyl is CH₂ and X is pyrazol (Compound 16). In some aspects, n is 5, alkyl is CH₂ and X is triazol (Compound 17).

In some aspects, an IP4-4,6 substituted derivative of the present invention comprises or consists of a compound of the following formula:

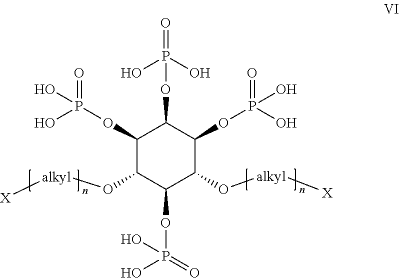

wherein n is an integer between 1 and 20, alkyl is CH₂ and X is —COOH. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt or decasodium salt. In some aspects, n is 5, alkyl is CH₂ and X is —COOH (Compound 18). In some aspects, n is 10, alkyl is CH₂ and X is —COOH (Compound 19). In some aspects, n is 3, alkyl is CH₂ and X is —COOH (Compound 52).

In some aspects, an IP4-4,6 substituted derivative of the present invention comprises or consists of a compound of the following formula:

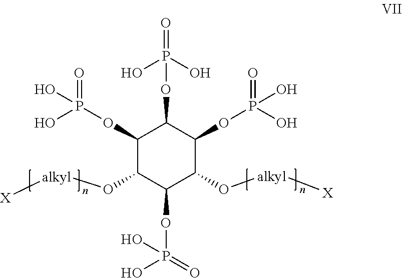

wherein n is an integer between 1 and 20, alkyl is CH₂ and X is —CONRR′, —NHCOR, —NHCOOR, —NHCONRR′ or —CF₃. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, n is 3, alkyl is CH₂ and X is —CF₃ (Compound 22). In some aspects, n is 5, alkyl is CH₂ and X is —CF₃ (Compound 23). In some aspects, n is 3, alkyl is CH₂ and X is —NHCOOMe (Compound 24). In some aspects, n is 5, alkyl is CH₂ and X is —NHCOOMe (Compound 25). In some aspects, n is 3, alkyl is CH₂ and X is —CONH₂ (Compound 26). In some aspects, n is 5, alkyl is CH₂ and X is —NHCOMe (Compound 27). In some aspects, n is 5, alkyl is CH₂ and X is —NHCONHPr (Compound 30).

In some aspects, an IP4-4,6 substituted derivative of the present invention comprises or consists of a compound of the following formula:

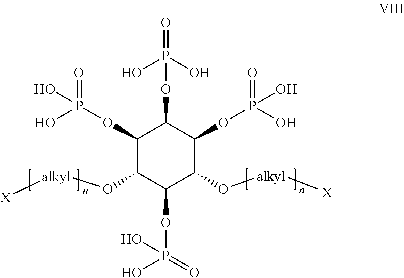

wherein n is an integer between 1 and 20, alkyl is CH₂ and X is cyclopropane or cyclopentane. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, n is 2, alkyl is CH₂ and X is cyclopropane (Compound 28). In some aspects, n is 2, alkyl is CH₂ and X is cyclopentane (Compound 29).

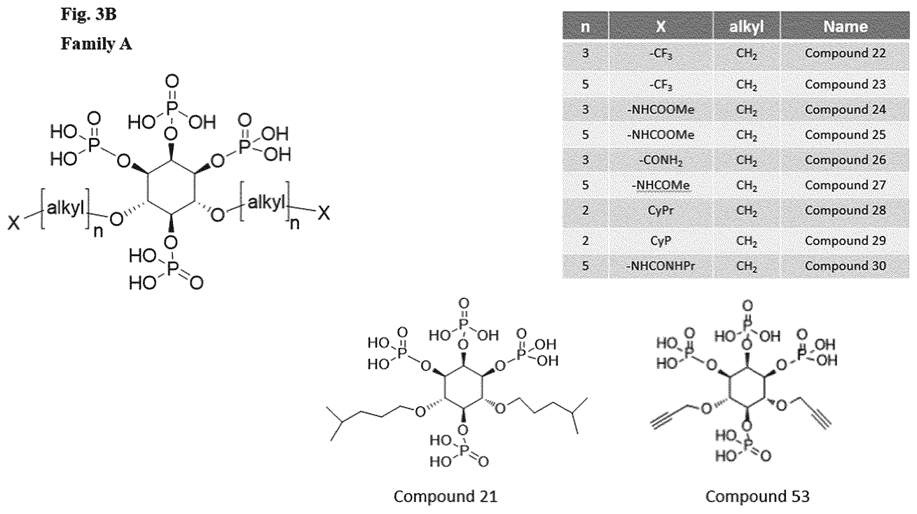

In some aspects, an IP4-4,6 substituted derivative of the present invention comprises or consists of a compound of the following formula:

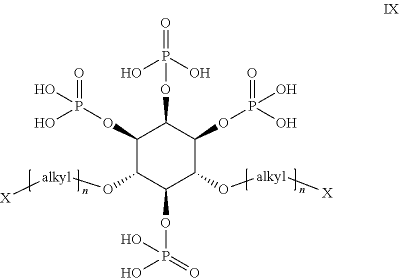

wherein n is an integer between 1 and 20, alkyl is branched chain and X is —H. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, an IP4-4,6 substituted derivative of the present invention comprises or consists of Compound 21 as shown in FIG. 3B.

In some aspects, an IP4-4,6 substituted derivative of the present invention comprises or consists of a compound of the following formula:

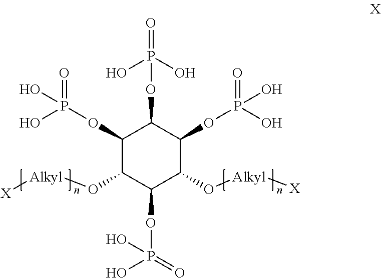

wherein n is an integer between 1 and 20, alkyl is an alkynyl chain and X is —H. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, an IP4-4,6 substituted derivative of the present invention comprises or consists of Compound 53 as shown in FIG. 3B.

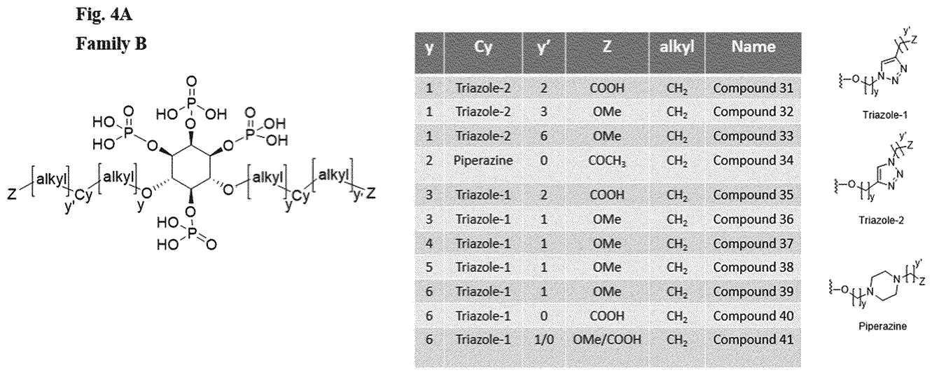

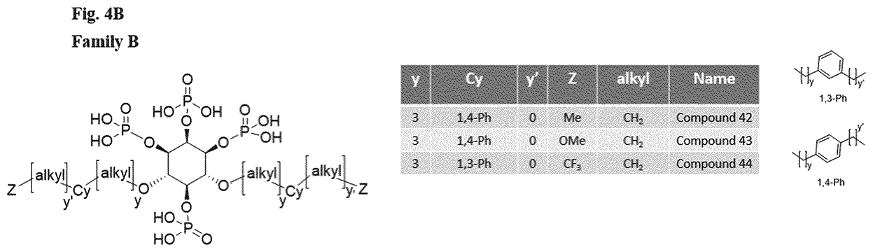

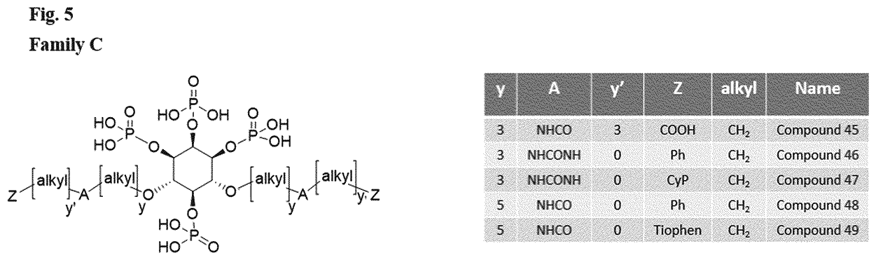

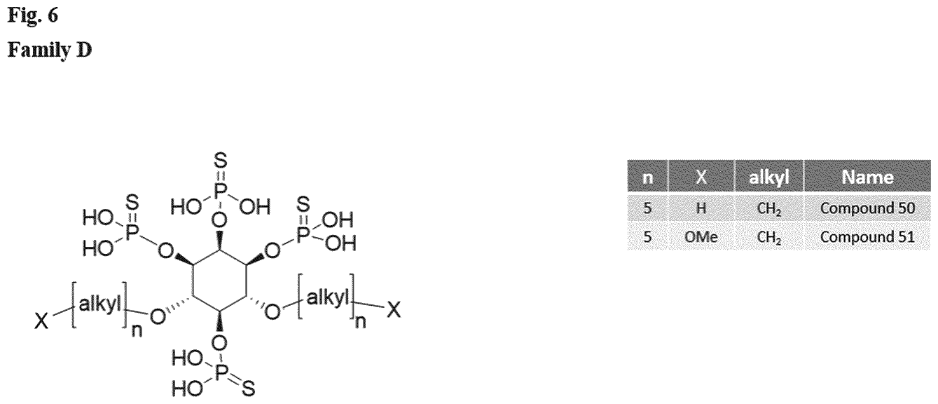

In some aspects, an IP4-4,6 substituted derivative of the present invention comprises or consists of a compound of the following formula:

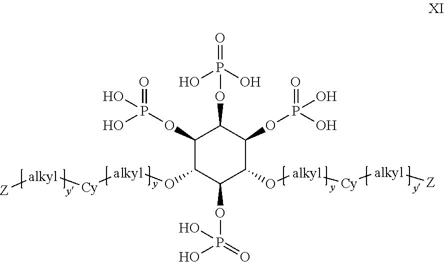

wherein y and y′ is an integer between 0 and 10, alkyl is CH₂, Cy is selected from the group consisting of piperazine, triazole-1, and triazole-2, and Z is selected from the group consisting of —COOH, —OMe, and —COCH₃. See FIG. 4A. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt or nonasodium salt. In some aspects, y or y′ is 0, 1, 2, 3, 4, 5, 6, 7, 8, 9 or 10. In some aspects, y is 1, y′ is 2, alkyl is CH₂, Cy is triazole-2, and Z is —COOH (Compound 31). In some aspects, y is 1, y′ is 3, alkyl is CH₂, Cy is triazole-2, and Z is —OMe (Compound 32). In some aspects, y is 1, y′ is 6, alkyl is CH₂, Cy is triazole-2, and Z is —OMe (Compound 33). In some aspects, y is 2, y′ is 0, alkyl is CH₂, Cy is piperazine, and Z is —COCH₃ (Compound 34). In some aspects, y is 3, y′ is 2, alkyl is CH₂, Cy is triazole-1, and Z is —COOH (Compound 35). In some aspects, y is 3, y′ is 1, alkyl is CH₂, Cy is triazole-1, and Z is —OMe (Compound 36). In some aspects, y is 4, y′ is 1, alkyl is CH₂, Cy is triazole-1, and Z is —OMe (Compound 37). In some aspects, y is 5, y′ is 1, alkyl is CH₂, Cy is triazole-1, and Z is —OMe (Compound 38). In some aspects, y is 6, y′ is 1, alkyl is CH₂, Cy is triazole-1, and Z is —OMe (Compound 39). In some aspects, y is 6, y′ is 0, alkyl is CH₂, Cy is triazole-1, and Z is —COOH (Compound 40). In some aspects, y is 6, y′ is 1/0, alkyl is CH₂, Cy is triazole-1, and Z is —OMe/—COOH (Compound 41).

In some aspects, an IP4-4,6 substituted derivative of the present invention comprises or consists of a compound of the following formula:

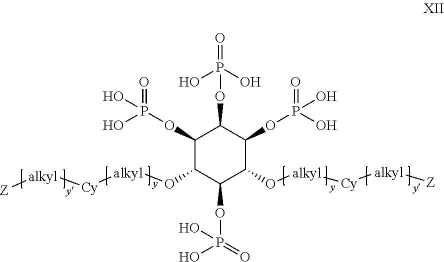

wherein y and y′ is an integer between 0 and 10, alkyl is CH₂, Cy is selected from the group consisting of 1,3-substituted phenyl and 1,4-substituted phenyl and Z is selected from the group consisting of -Me, —OMe, and —CF₃. See FIG. 4B. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, y or y′ is 0, 1, 2, 3, 4, 5, 6, 7, 8, 9 or 10. In some aspects, y is 3, y′ is 0, alkyl is CH₂, Cy is 1,4-substituted phenyl, and Z is -Me (Compound 42). In some aspects, y is 3, y′ is 0, alkyl is CH₂, Cy is 1,4-substituted phenyl, and Z is —OMe (Compound 43). In some aspects, y is 3, y′ is 0, alkyl is CH₂, Cy is 1,3-substituted phenyl, and Z is —CF₃ (Compound 44).

In some aspects, an IP4-4,6 substituted derivative of the present invention comprises or consists of a compound of the following formula:

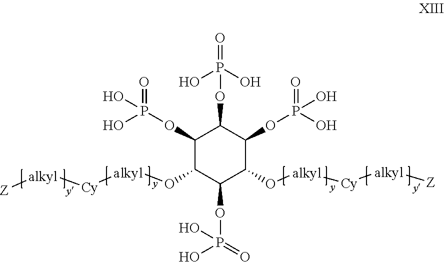

wherein y and y′ is an integer between 0 and 10, alkyl is CH₂, A is selected from the group consisting of —NHCO and —NHCONH, and Z is selected from the group consisting of —COOH, phenyl, cyclopentyl, and thiophenyl. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt or decasodium salt. In some aspects, y or y′ is 0, 1, 2, 3, 4, 5, 6, 7, 8, 9 or 10. In some aspects, y is 3, y′ is 3, alkyl is CH₂, A is —NHCO, and Z is —COOH (Compound 45). In some aspects, y is 3, y′ is 0, alkyl is CH₂, A is —NHCONH, and Z is phenyl (Compound 46). In some aspects, y is 3, y′ is 0, alkyl is CH₂, A is —NHCONH, and Z is cyclopentyl (Compound 47). In some aspects, y is 5, y′ is 0, alkyl is CH₂, A is —NHCO, and Z is phenyl (Compound 48). In some aspects, y is 5, y′ is 0, alkyl is CH₂, A is —NHCO, and Z is thiophenyl (Compound 49).

In some aspects, an IP4-4,6 substituted derivative of the present invention comprises or consists of a compound of the following formula:

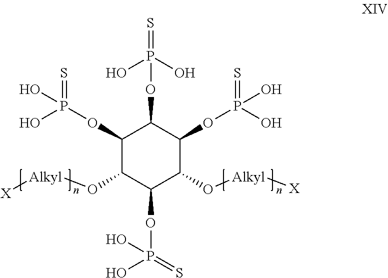

wherein n is an integer between 1 and 20, alkyl is CH₂ and X is selected from the group consisting of —H and —OMe. In some aspects, n is 1, 2, 3, 4, 5, 6, 7, 8, 9, 10, 11, 12, 13, 14, 15, 16, 17, 18, 19 or 20. In some aspects, the IP4-4,6 derivative is a sodium salt. In some aspects, the IP4-4,6 derivative is a tetrasodium salt, pentasodium salt, hexasodium salt, heptasodium salt, octasodium salt, nonasodium salt or decasodium salt. In some aspects, the IP4-4,6 derivative is a tetrasodium salt, pentasodium salt, hexasodium salt, heptasodium salt or octasodium salt. In some aspects, the IP4-4,6 derivative is a hexasodium salt, octasodium salt, nonasodium salt or decasodium salt. In some aspects, the IP4-4,6 derivative is an octasodium salt. In some aspects, n is 5, alkyl is CH₂ and X is —H (Compound 50). In some aspects, n is 5, alkyl is CH₂ and X is —OMe (Compound 51).

The IP4-4,6 derivatives of the present invention are disclosed in myo form. However, the invention is also meant to encompass all the other isomers of the inositol scaffold, such as the scyllo, muco, 1D-chiro, 1L-chiro, neo, allo, epi, and cis counterparts of the IP4-4,6 derivatives of the invention.

The present invention also provides chemical intermediate compounds useful in the preparations of IP4-4,6 substituted derivative of the present invention (e.g., Compound 1 to Compound 53). In some aspects, such intermediates are the compounds listed in Table 1. An intermediate compound disclosed herein can be converted to an IP4-4,6 substituted derivative of the present invention by utilizing the procedures described herein. Thus, the present invention provides methods to produce an IP4-4,6 substituted derivative of the present invention (e.g., a compound selected from the group consisting of Compounds 1 to 53) comprising utilizing an intermediate compound selected from the group consisting of the compounds listed in Table 1. The present invention also provides methods of producing the intermediate compounds disclosed herein. Accordingly, the present invention provides methods of producing intermediates compounds selected from the group consisting of the compounds listed in Table 1 for producing IP4-4,6 substituted derivatives of the present invention (e.g., a compound selected from the group consisting of Compounds 1 to 53).

TABLE 1
Intermediates

| Formula | Intermediate |
|---|---|
| II-1 | 4,6-O-bis(5-(Benzyloxy)pentyl)-2-O-tert-butyldimethylsilyl-1,3,5-O-methylidyne- |
| | myo-inositol |
| II-2 | 4,6-O-bis(10-(Benzyloxy)decyl)-2-O-tert-butyldimethylsilyl-1,3,5-O-methylidyne- |
| | myo-inositol |
| II-3 | 4,6-O-bis(14-(Benzyloxy)tetradecyl)-2-O-tert-butyldimethylsilyl-1,3,5-O- |
| | methylidyne-myo-inositol |
| II-4 | 2-O-tert-Butyldimethylsilyl-4,6-O-bis(3-methoxypropyl)-1,3,5-O-methylidyne-myo- |
| | inositol |
| II-5 | 2-O-tert-Butyldimethylsilyl-4,6-O-bis(5-methoxypentyl)-1,3,5-O-methylidyne-myo- |
| | inositol |
| II-6 | 2-O-tert-Butyldimethylsilyl-4,6-O-bis(9-methoxynonyl)-1,3,5-O-methylidyne-myo- |
| | inositol |
| II-7 | 2-O-tert-Butyldimethylsilyl-4,6-O-bis(7-methoxyheptyl)-1,3,5-O-methylidyne-myo- |
| | inositol |
| II-8 | 2-O-tert-Butyldimethylsilyl-1,3,5-O-methylidyne-4,6-O-dipropyl-myo-inositol |
| II-9 | 2-O-tert-Butyldimethylsilyl-1,3,5-O-methylidyne-4,6-O-dipentyl-myo-inositol |
| II-10 | 2-O-tert-Butyldimethylsilyl-4,6-O-diheptyl-1,3,5-O-methylidyne-myo-inositol |
| II-11 | 2-O-tert-Butyldimethylsilyl-4,6-O-didecyl-1,3,5-O-methylidyne-myo-inositol |
| II-12 | 2-O-tert-Butyldimethylsilyl-1,3,5-O-methylidyne-4,6-O-di(tetradecyl)-myo-inositol |
| II-13 | 2-O-tert-Butyldimethylsilyl-4,6-O-bis(5-ethoxycarbonylpentyl)-1,3,5-O- |
| | methylidyne-myo-inositol |
| II-14 | 2-O-tert-Butyldimethylsilyl-4,6-O-bis(10-ethoxycarbonyldecyl)-1,3,5-O- |
| | methylidyne-myo-inositol |
| II-15 | 2-O-tert-Butyldimethylsilyl-1,3,5-O-methylidyne-4,6-O-bis(5-(1H-pyrazol-1- |
| | yl)pentyl)-myo-inositol |
| II-16 | 2-O-tert-Butyldimethylsilyl-1,3,5-O-methylidyne-4,6-O-bis(5-(1H-1,2,4-triazol-1- |
| | yl)pentyl)-myo-inositol |
| II-17 | 4,6-O-bis(2-(4-Acetylpiperazin-1-yl)ethyl)-2-O-tert-butyldimethylsilyl-1,3,5-O- |
| | methylidyne-myo-inositol |
| II-18 | 2-O-tert-Butyldimethylsilyl-1,3,5-O-methylidyne-4,6-O-bis(4,4,4-trifluorobutyl)- |
| | myo-inositol |
| II-19 | 2-O-tert-Butyldimethylsilyl-1,3,5-O-methylidyne-4,6-O-bis(6,6,6-trifluorohexyl)- |
| | myo-inositol |
| II-20 | 2-O-tert-Butyldimethylsilyl-1,3,5-O-methylidyne-4,6-O-bis(4-methylpentyl)-myo- |
| | inositol |
| II-21 | 2-O-tert-Butyldimethylsilyl-4,6-O-bis(19-methoxynonadec-10-yn-1-yl)-1,3,5-O- |
| | methylidyne-myo-inositol |
| II-22 | 2-O-tert-Butyldimethylsilyl-4,6-O-bis(2-cyclopropylethy)-1,3,5-O-methylidyne- |
| | myo-inositol |
| II-23 | 2-O-tert-Butyldimethylsilyl-4,6-O-bis(2-cyclopentylethy)-1,3,5-O-methylidyne- |
| | myo-inositol |
| II-24 | 2-O-tert-Butyldimethylsilyl-4,6-O-bis(3-(4-methoxyphenyl)propyl)-1,3,5-O- |
| | methylidyne-myo-inositol |
| II-25 | 2-O-tert-Butyldimethylsilyl-1,3,5-O-methylidyne-4,6-O-bis(3-(3- |
| | (trifluoromethyl)phenyl)propyl)-myo-inositol |
| II-26 | 2-O-tert-Butyldimethylsilyl-1,3,5-O-methylidyne-4,6-O-bis(3-(p-tolyl)propyl)-myo- |
| | inositol |
| II-27 | 2-O-tert-Butyldimethylsilyl-4,6-O-bis(3-methoxycarbonylproyl)-1,3,5-O- |
| | methylidyne-myo-inositol |
| III-1 | 4,6-O-bis(5-(Benzyloxy)pentyl)-myo-inositol |
| III-2 | 4,6-O-bis(5-Methoxypentyl)-myo-inositol |
| III-3 | 4,6-O-bis(10-(Benzyloxy)decyl)-myo-inositol |
| III-4 | 4,6-O-bis(14-(Benzyloxy)tetradecyl)-myo-inositol |
| III-5 | 4,6-O-bis(5-Ethoxycarbonylpentyl)-myo-inositol |
| III-6 | 4,6-O-bis(10-Ethoxycarbonyldecyl)-myo-inositol |
| III-7 | 4,6-O-bis(2-cyclopropylethy)-myo-inositol |
| III-8 | 4,6-O-bis(2-cyclopentylethy)-myo-inositol |
| III-9 | 4,6-O-bis(5-(1H-Pyrazol-1-yl)pentyl)-myo-inositol |
| III-10 | 4,6-O-bis(5-(1H-1,2,4-Triazol-1-yl)pentyl)-myo-inositol |
| III-11 | 4,6-O-bis(2-(4-Acetylpiperazin-1-yl)ethyl)-myo-inositol |
| III-12 | 4,6-O-bis(3-Methoxypropyl)-myo-inositol |
| III-13 | 4,6-O-bis(7-Methoxyheptyl)-myo-inositol |
| III-14 | 4,6-O-bis(9-Methoxynonyl)-myo-inositol |
| III-15 | 4,6-O-Dipropyl-myo-inositol |
| III-16 | 4,6-O-Dipentyl-myo-inositol |
| III-17 | 4,6-O-Diheptyl-myo-inositol |
| III-18 | 4,6-O-Didecyl-myo-inositol |
| III-19 | 4,6-O-Di(tetradecyl)-myo-inositol |
| III-20 | 4,6-O-bis((1-(2-(Benzyloxycarbonyl)ethyl)-1H-1,2,3-triazol-4-yl)methyl)-myo- |
| | inositol |
| III-21 | 4,6-O-bis(3-(4-(2-(Benzyloxycarbonyl)ethyl)-1H-1,2,3-triazol-1-yl)propyl)-myo- |
| | inositol |
| III-22 | 4,6-O-bis(3-(4-(Methoxymethyl)-1H-1,2,3-triazol-1-yl)propyl)-myo-inositol |
| III-23 | 4,6-O-bis(4-(4-(Methoxymethyl)-1H-1,2,3-triazol-1-yl)butyl)-myo-inositol |
| III-24 | 4,6-O-bis(5-(4-(Methoxymethyl)-1H-1,2,3-triazol-1-yl)pentyl)-myo-inositol |
| III-25 | 4,6-O-bis(6-(4-(Methoxymethyl)-1H-1,2,3-triazol-1-yl)hexyl)-myo-inositol |
| III-26 | 4,6-O-bis((1-(3-Methoxypropyl)-1H-1,2,3-triazol-4-yl)methyl)-myo-inositol |
| III-27 | 4,6-O-bis((1-(6-Methoxyhexyl)-1H-1,2,3-triazol-4-yl)methyl)-myo-inositol |
| III-28 | 4,6-O-bis((3-(4-Ethyloxycarbonylbutanamido)propyl))-myo-inositol |
| III-29 | 4,6-O-bis(3-(4-methoxyphenyl)propyl)-myo-inositol |
| III-30 | 4,6-O-bis(3-(3-(trifluoromethyl)phenyl)propyl)-myo-inositol |
| III-31 | 4,6-O-bis(3-(p-tolyl)propyl)-myo-inositol |
| III-32 | 4,6-O-bis(4,4,4-trifluorobutyl)-myo-inositol |
| III-33 | 4,6-O-bis(6,6,6-trifluorohexyl)-myo-inositol |
| III-34 | 4,6-O-bis(4-methylpentyl)-myo-inositol |
| III-35 | 4,6-O-bis(19-methoxynonadec-10-yn-1-yl)-myo-inositol |
| III-36 | 4,6-O-bis(3-(3-phenylureido)propyl)-myo-inositol |
| III-37 | 4,6-O-bis(3-(3-cyclopentylureido)propyl)-myo-inositol |
| III-38 | 4,6-O-bis(3-((methoxycarbonyl)amino)propyl)-myo-inositol |
| III-39 | 4,6-O-bis(5-acetamidopentyl)-myo-inositol |
| III-40 | 4,6-O-bis(5-benzamidopentyl)-myo-inositol |
| III-41 | 4,6-O-bis(5-(thiophene-2-carboxamido)pentyl)-myo-inositol |
| III-42 | 4,6-O-(6-(4-(methoxycarbonyl)-1H-1,2,3-triazol-1-yl)hexyl)-myo-inositol |
| III-43 | 4-O-(6-(4-(methoxycarbonyl)-1H-1,2,3-triazol-1-yl)hexyl)-6-O-(6-(4- |
| | (Methoxymethyl)-1H-1,2,3-triazol-1-yl)hexyl)-myo-inositol |
| III-44 | 4,6-O-bis(4-amino-4-oxobutyl)-myo-inositol |
| III-45 | 4,6-O-bis(5-(3-propylureido)pentyl)-myo-inositol |
| III-46 | 4,6-O-bis(5-((methoxycarbonyl)amino)pentyl)-myo-inositol |
| III-47 | 4,6-O-bis(3-Methoxycarbonylpropyl)-myo-inositol |
| IV-1 | 4,6-O-bis(10-(Benzyloxy)decyl)-1,2,3,5-O-tetrakis(3-oxido-1,5- |
| | dihydrobenzo[e][1,3,2]dioxaphosphepin-3-yl)-myo-inositol |
| IV-2 | 4,6-O-bis(14-(Benzyloxy)tetradecyl)-1,2,3,5-O-tetrakis(3-oxido-1,5- |
| | dihydrobenzo[e][1,3,2]dioxaphosphepin-3-yl)-myo-inositol |
| IV-3 | 4,6-O-bis(3-Methoxypropyl)-1,2,3,5-O-tetrakis(3-oxido-1,5- |
| | dihydrobenzo[e][1,3,2]dioxaphosphepin-3-yl)-myo-inositol |
| IV-4 | 4,6-O-bis(5-Methoxypentyl)-1,2,3,5-O-tetrakis(3-oxido-1,5- |
| | dihydrobenzo[e][1,3,2]dioxaphosphepin-3-yl)-myo-inositol |
| IV-5 | 4,6-O-bis(7-Methoxyheptyl)-1,2,3,5-O-tetrakis(3-oxido-1,5- |
| | dihydrobenzo[e][1,3,2]dioxaphosphepin-3-yl)-myo-inositol |
| IV-6 | 4,6-O-bis(7-Methoxynonyl)-1,2,3,5-O-tetrakis(3-oxido-1,5- |
| | dihydrobenzo[e][1,3,2]dioxaphosphepin-3-yl)-myo-inositol |
| IV-7 | 1,2,3,5-O-tetrakis(3-Oxido-1,5-dihydrobenzo[e][1,3,2]dioxaphosphepin-3-yl)- 4,6- |
| | O-dipropyl-myo-inositol |
| IV-8 | 1,2,3,5-O-tetrakis(3-Oxido-1,5-dihydrobenzo[e][1,3,2]dioxaphosphepin-3-yl)- 4,6- |
| | O-dipentyl-myo-inositol |
| IV-9 | 4,6-O-Diheptyl-1,2,3,5-O-tetrakis(3-oxido-1,5- |
| | dihydrobenzo[e][1,3,2]dioxaphosphepin-3-yl)-myo-inositol |
| IV-10 | 4,6-O-Didecyl-1,2,3,5-O-tetrakis(3-oxido-1,5- |
| | dihydrobenzo[e][1,3,2]dioxaphosphepin-3-yl)-myo-inositol |
| IV-11 | 1,2,3,5-O-tetrakis(3-Oxido-1,5-dihydrobenzo[e][1,3,2]dioxaphosphepin-3-yl)- 4,6- |
| | O-di(tetradecyl)-myo-inositol |
| IV-12 | 4,6-O-bis(5-Ethoxycarbonylpentyl)-1,2,3,5-O-tetrakis(3-oxido-1,5- |
| | dihydrobenzo[e][1,3,2]dioxaphosphepin-3-yl)-myo-inositol |
| IV-13 | 4,6-O-bis(10-Ethoxycarbonyldecyl)-1,2,3,5-O-tetrakis(3-oxido-1,5- |
| | dihydrobenzo[e][1,3,2]dioxaphosphepin-3-yl)-myo-inositol |
| IV-14 | 1,2,3,5-O-tetrakis(3-Oxido-1,5-dihydrobenzo[e][1,3,2]dioxaphosphepin-3-yl)-4,6-O- |
| | bis(5-(1H-pyrazol-1-yl)pentyl)-myo-inositol |
| IV-15 | 1,2,3,5-O-tetrakis(3-Oxido-1,5-dihydrobenzo[e][1,3,2]dioxaphosphepin-3-yl)-4,6-O- |
| | bis(5-(1H-1,2,4-triazol-1-yl)pentyl)-myo-inositol |
| IV-16 | 4,6-O-bis((1-(2-(Benzyloxycarbonyl)ethyl)-1H-1,2,3-triazol-4-yl)methyl)-1,2,3,5-O- |
| | tetrakis(3-oxido-1,5-dihydrobenzo[e][1,3,2]dioxaphosphepin-3-yl)-myo-inositol |
| IV-17 | 4,6-O-bis((1-(3-Methoxypropyl)-1H-1,2,3-triazol-4-yl)methyl)-1,2,3,5-O-tetrakis(3- |
| | oxido-1,5-dihydrobenzo[e][1,3,2]dioxaphosphepin-3-yl)-myo-inositol |
| IV-18 | 4,6-O-bis((1-(6-Methoxyhexyl)-1H-1,2,3-triazol-4-yl)methyl)-1,2,3,5-O-tetrakis(3- |
| | oxido-1,5-dihydrobenzo[e][1,3,2]dioxaphosphepin-3-yl)-myo-inositol |
| IV-19 | 4,6-O-bis(3-(4-(2-(Benzyloxycarbonyl)ethyl)-1H-1,2,3-triazol-1-yl)propyl)-1,2,3,5- |
| | O-tetrakis(3-oxido-1,5-dihydrobenzo[e][1,3,2]dioxaphosphepin-3-yl)-myo-inositol |
| IV-20 | 4,6-O-bis(3-(4-(Methoxymethyl)-1H-1,2,3-triazol-1-yl)propyl)-1,2,3,5-O-tetrakis(3- |
| | oxido-1,5-dihydrobenzo[e][1,3,2]dioxaphosphepin-3-yl)-myo-inositol |
| IV-21 | 4,6-O-bis(4-(4-(Methoxymethyl)-1H-1,2,3-triazol-1-yl)butyl)-1,2,3,5-O-tetrakis(3- |
| | oxido-1,5-dihydrobenzo[e][1,3,2]dioxaphosphepin-3-yl)-myo-inositol |
| IV-22 | 4,6-O-bis(5-(4-(Methoxymethyl)-1H-1,2,3-triazol-1-yl)pentyl)-1,2,3,5-O-tetrakis(3- |
| | oxido-1,5-dihydrobenzo[e][1,3,2]dioxaphosphepin-3-yl)-myo-inositol |
| IV-23 | 4,6-O-bis(6-(4-(Methoxymethyl)-1H-1,2,3-triazol-1-yl)hexyl)-1,2,3,5-O-tetrakis(3- |
| | oxido-1,5-dihydrobenzo[e][1,3,2]dioxaphosphepin-3-yl)-myo-inositol |
| IV-24 | 4,6-O-bis(2-(4-Acetylpiperazin-1-yl)ethyl)-1,2,3,5-O-tetrakis(3-oxido-1,5- |
| | dihydrobenzo[e][1,3,2]dioxaphosphepin-3-yl)-myo-inositol |
| IV-25 | 4,6-O-bis((3-(4-Ethyloxycarbonylbutanamido)propyl))-1,2,3,5-O-tetrakis(3-oxido- |
| | 1,5-dihydrobenzo[e][1,3,2]dioxaphosphepin-3-yl)-myo-inositol |
| IV-26 | 4,6-O-bis(5-(Benzyloxy)pentyl)-1,2,3,5-O-tetrakis(3-oxido-1,5- |
| | dihydrobenzo[e][1,3,2]dioxaphosphepin-3-yl)-myo-inositol |
| IV-27 | 4,6-O-bis(2-cyclopropylethy)-1,2,3,5-O-tetrakis(3-oxido-1,5- |
| | dihydrobenzo[e][1,3,2]dioxaphosphepin-3-yl)-myo-inositol |
| IV-28 | 4,6-O-bis(2-cyclopentylethy)-1,2,3,5-O-tetrakis(3-oxido-1,5- |
| | dihydrobenzo[e][1,3,2]dioxaphosphepin-3-yl)-myo-inositol |
| IV-29 | 4,6-O-bis(3-(4-methoxyphenyl)propyl)-1,2,3,5-O-tetrakis(3-oxido-1,5- |
| | dihydrobenzo[e][1,3,2]dioxaphosphepin-3-yl)-myo-inositol |
| IV-30 | 1,2,3,5-O-tetrakis(3-oxido-1,5-dihydrobenzo[e][1,3,2]dioxaphosphepin-3-yl)-4,6-O- |
| | bis(3-(3-(trifluoromethyl)phenyl)propyl)-myo-inositol |
| IV-31 | 1,2,3,5-O-tetrakis(3-oxido-1,5-dihydrobenzo[e][1,3,2]dioxaphosphepin-3-yl)-4,6-O- |
| | bis(3-(p-tolyl)propyl)-myo-inositol |
| IV-32 | 1,2,3,5-O-tetrakis(3-oxido-1,5-dihydrobenzo[e][1,3,2]dioxaphosphepin-3-yl)-4,6-O- |
| | bis(4,4,4-trifluorobutyl)-myo-inositol |
| IV-33 | 1,2,3,5-O-tetrakis(3-oxido-1,5-dihydrobenzo[e][1,3,2]dioxaphosphepin-3-yl)-4,6-O- |
| | bis(6,6,6-trifluorohexyl)-myo-inositol |
| IV-34 | 4,6-O-bis(4-Methylpentyl)-1,2,3,5-O-tetrakis(3-oxido-1,5- |
| | dihydrobenzo[e][1,3,2]dioxaphosphepin-3-yl)-myo-inositol |
| IV-35 | 4,6-O-bis(19-Methoxynonadec-10-yn-1-yl)-1,2,3,5-O-tetrakis(3-oxido-1,5- |
| | dihydrobenzo[e][1,3,2]dioxaphosphepin-3-yl)-myo-inositol |
| IV-36 | 1,2,3,5-O-tetrakis(3-oxido-1,5-dihydrobenzo[e][1,3,2]dioxaphosphepin-3-yl)-4,6-0- |
| | bis(3-(3-phenylureido)propyl)-myo-inositol |
| IV-37 | 4,6-O-bis(3-(3-cyclopentylureido)propyl)-1,2,3,5-O-tetrakis(3-oxido-1,5- |
| | dihydrobenzo[e][1,3,2]dioxaphosphepin-3-yl)-myo-inositol |
| IV-38 | 4,6-O-bis(3-((methoxycarbonyl)amino)propyl)-1,2,3,5-O-tetrakis(3-oxido-1,5- |
| | dihydrobenzo[e][1,3,2]dioxaphosphepin-3-yl)-myo-inositol |
| IV-39 | 4,6-O-bis(5-acetamidopentyl)-1,2,3,5-O-tetrakis(3-oxido-1,5- |
| | dihydrobenzo[e][1,3,2]dioxaphosphepin-3-yl)-myo-inositol |
| IV-40 | 4,6-O-bis(5-benzamidopentyl)-1,2,3,5-O-tetrakis(3-oxido-1,5- |
| | dihydrobenzo[e][1,3,2]dioxaphosphepin-3-yl)-myo-inositol |
| IV-41 | 1,2,3,5-O-tetrakis(3-oxido-1,5-dihydrobenzo[e][1,3,2]dioxaphosphepin-3-yl)-4,6-O- |
| | bis(5-(thiophene-2-carboxamido)pentyl)-myo-inositol |
| IV-42 | 4,6-O-(6-(4-(methoxycarbonyl)-1H-1,2,3-triazol-1-yl)hexyl)-1,2,3,5-O-tetrakis(3- |
| | oxido-1,5-dihydrobenzo[e][1,3,2]dioxaphosphepin-3-yl)-myo-inositol |
| IV-43 | 4-O-(6-(4-(methoxycarbonyl)-1H-1,2,3-triazol-1-yl)hexyl)-6-O-(6-(4- |
| | (Methoxymethyl)-1H-1,2,3-triazol-1-yl)hexyl)-1,2,3,5-O-tetrakis(3-oxido-1,5- |
| | dihydrobenzo[e][1,3,2]dioxaphosphepin-3-yl)-myo-inositol |
| IV-44 | 4,6-O-bis(4-amino-4-oxobutyl)-1,2,3,5-O-tetrakis(3-oxido-1,5- |
| | dihydrobenzo[e][1,3,2]dioxaphosphepin-3-yl)-myo-inositol |
| IV-45 | 1,2,3,5-O-tetrakis(3-oxido-1,5-dihydrobenzo[e][1,3,2]dioxaphosphepin-3-yl)-4,6-O- |
| | bis(5-(3-propylureido)pentyl)-myo-inositol |
| IV-46 | 4,6-O-bis(5-((methoxycarbonyl)amino)pentyl)-1,2,3,5-O-tetrakis(3-oxido-1,5- |
| | dihydrobenzo[e][1,3,2]dioxaphosphepin-3-yl)-myo-inositol |
| IV-47 | 4,6-O-bis(3-methoxycarbonylpropyl)-1,2,3,5-O-tetrakis(3-oxido-1,5- |
| | dihydrobenzo[e][1,3,2]dioxaphosphepin-3-yl)-myo-inositol |
| IV-48 | 4,6-O-Dipropargyl-1,2,3,5-O-tetrakis(3-oxido-1,5- |
| | dihydrobenzo[e][1,3,2]dioxaphosphepin-3-yl)-myo-inositol |
| IV′-1 | 4,6-O-dipentyl-1,2,3,5-O-tetrakis(3-sulfido-1,5- |
| | dihydrobenzo[e][1,3,2]dioxaphosphepin-3-yl)-myo-inositol |
| IV′-2 | 4,6-O-bis(5-Methoxypentyl)-1,2,3,5-O-tetrakis(3-sulfido-1,5- |
| | dihydrobenzo[e][1,3,2]dioxaphosphepin-3-yl)-myo-inositol |
| V-1 | 2-O-tert-Butyldimethylsilyl-1,3,5-O-methylidyne-4,6-O-dipropargyl-myo-inositol |
| VI-1 | 4,6-O-Dipropargyl-myo-inositol |
| VII-1 | 4,6-O-bis((1-(3-(Tosyloxy)propyl)-1H-1,2,3-triazol-4-yl)methyl)-myo-inositol |
| VII-2 | 4,6-O-bis((1-(6-(Tosyloxy)hexyl)-1H-1,2,3-triazol-4-yl)methyl)-myo-inositol |
| VIII-1 | 4,6-O-bis(3-Azidopropyl)-2-O-tert-butyldimethylsilyl-1,3,5-O-methylidyne-myo- |
| | inositol |
| VIII-2 | 4,6-O-bis(5-Azidopentyl)-2-O-tert-butyldimethylsilyl-1,3,5-O-methylidyne-myo- |
| | inositol |
| VIII-3 | 4,6-O-bis(10-Azidodecyl)-2-O-tert-butyldimethylsilyl-1,3,5-O-methylidyne-myo- |
| | inositol |
| VIII-4 | 4,6-O-bis(4-Azidobutyl)-2-O-tert-butyldimethylsilyl-1,3,5-O-methylidyne-myo- |
| | inositol |
| VIII-5 | 4,6-O-bis(6-Azidohexyl)-2-O-tert-butyldimethylsilyl-1,3,5-O-methylidyne-myo- |
| | inositol |
| IX-1 | 4,6-O-bis(4-Azidobutyl)-myo-inositol |
| IX-2 | 4,6-O-bis(6-Azidohexyl)-myo-inositol |
| IX-3 | 4,6-O-bis(3-Azidopropyl)-myo-inositol |
| IX-4 | 4,6-O-bis(5-Azidopentyl)-myo-inositol |
| IX-5 | 4,6-O-bis(10-Azidodecyl)-myo-inositol |
| X-1 | 4,6-O-bis(3-Azidopropyl)-1,2,3,5-O-tetrakis(3-oxido-1,5- |
| | dihydrobenzo[e][1,3,2]dioxaphosphepin-3-yl)-myo-inositol |
| X-2 | 4,6-O-bis(5-Azidopentyl)-1,2,3,5-O-tetrakis(3-oxido-1,5- |
| | dihydrobenzo[e][1,3,2]dioxaphosphepin-3-yl)-myo-inositol |
| X-3 | 4,6-O-bis(10-Azidodecyl)-1,2,3,5-O-tetrakis(3-oxido-1,5- |
| | dihydrobenzo[e][1,3,2]dioxaphosphepin-3-yl)-myo-inositol |
| XI-1 | 4,6-O-bis(3-Aminopropyl)-2-O-tert-butyldimethylsilyl-1,3,5-O-methylidyne-myo- |
| | inositol |
| XI-2 | 4,6-O-bis(5-Aminopentyl)-2-O-tert-butyldimethylsilyl-1,3,5-O-methylidyne-myo- |
| | inositol |
| XII-1 | 4,6-O-bis(3-Aminopropyl)-myo-inositol•2TFA |
| XII-2 | 4,6-O-bis(5-Aminopentyl)-2-O-tert-butyldimethylsilyl-1,3,5-O-methylidyne-myo- |
| | inositol |
| XIII-1 | 4,6-O-bis(5-acetamidopentyl)-2-O-tert-butyldimethylsilyl-1,3,5-O-methylidyne- |
| | myo-inositol |
| XIII-2 | 4,6-O-bis(5-benzamidopentyl)-2-O-tert-butyldimethylsilyl-1,3,5-O-methylidyne- |
| | myo-inositol |
| XIII-3 | 2-O-tert-butyldimethylsilyl-4,6-O-bis(5-((methoxycarbonyl)amino)pentyl)-1,3,5-O- |
| | methylidyne-myo-inositol |

IP4-4,6 substituted derivatives of the present invention and intermediates for their synthesis can be synthesized by using the methods described herein, as well as other processes known in the field of the organic chemistry. In some aspects, the methods include, but are not limited to, the general procedures shown in the synthesis Schemes 1, 2, 3, and 4 described herein. Thus, in some aspects, the present invention provides a method to manufacture an IP4-4,6 substituted derivative of the present invention comprising applying synthetic Scheme 1 disclosed below. In some aspects, the present invention provides a method to manufacture an IP4-4,6 substituted derivative of the present invention comprising applying synthetic Scheme 2 disclosed below. In some aspects, the present invention provides a method to manufacture an IP4-4,6 substituted derivative of the present invention comprising applying synthetic Scheme 3 disclosed below. In some aspects, the present invention provides a method to manufacture an IP4-4,6 substituted derivative of the present invention comprising applying synthetic Scheme 4 disclosed below.

In some aspects, the present invention provides a method to manufacture an intermediate for the synthesis of an IP4-4,6 substituted derivative of the present invention (e.g., a compound selected from the group consisting of Compound 1 to Compound 53) comprising applying synthetic Scheme 1 disclosed below. In some aspects, the present invention provides a method to manufacture an intermediate for the synthesis of an IP4-4,6 substituted derivative of the present invention (e.g., a compound selected from the group consisting of Compound 1 to Compound 53) comprising applying synthetic Scheme 2 disclosed below. In some aspects, the present invention provides a method to manufacture an intermediate for the synthesis of an IP4-4,6 substituted derivative of the present invention (e.g., a compound selected from the group consisting of Compound 1 to Compound 53) comprising applying synthetic Scheme 3 disclosed below. In some aspects, the present invention provides a method to manufacture an intermediate for the synthesis of an IP4-4,6 substituted derivative of the present invention (e.g., a compound selected from the group consisting of Compound 1 to Compound 53) comprising applying synthetic Scheme 4 disclosed below.

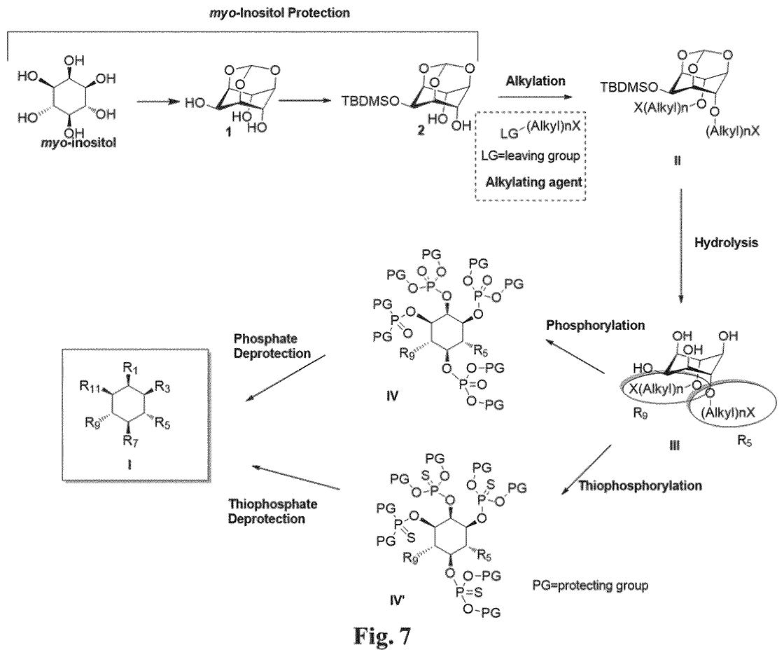

Scheme 1: IP4-4,6 substituted derivatives of the present invention can be obtained, e.g., by deprotection of intermediates of formula IV (e.g., Intermediates IV-1 to IV-48) and intermediates of formula IV′ (e.g., Intermediates IV′-1 to IV′-2). See Scheme 1, FIG. 7. The “protective group” or PG, can be, without limitation, benzyl, levulinylbenzyl, tert-butyl, o-xylenyl (e.g., by union of 2 PG in the same phosphate), 9-fluorenylmethyl, cyanethyl and other suitable protective groups in each case. Intermediates of formula IV (e.g., Intermediates IV-1 to IV-48) can be achieved, e.g., by phosphorylation of an intermediate of formula III according to procedures described in the literature, e.g., by a reaction with a phosphoroamidite derivative and subsequent oxidation with an oxidant (e.g., m-CPBA, H₂O₂, tBuOOH). Intermediates of formula IV′ (e.g., Intermediates IV′-1 to IV′-2) can be achieved, e.g., by thiophosphorylation of an intermediate of formula III according to procedures described in the art, e.g., by the reaction with a phosphoroamidite derivative and subsequent thio-oxidation with sulfur. At the same time, intermediates of formula III (e.g., Intermediates III-1 to III-47) can be obtained, e.g., by hydrolysis in acid media of intermediates of formula II (e.g., Intermediates II-1 to II-27). Finally, intermediates of formula II (e.g., Intermediates II-1 to II-27) can be obtained, e.g., by alkylation of compound (2) with al alkylating agent. Preparation of (2) was previously described in the literature (Martin S, et al., J. Org. Chem. 1994; 59:4805; Kardivel M, Org. & Biomolecular Chem. 2008; 6(11):1966-72). The “Leaving Group” or LG, may be, without limitation, chloride, bromide, iodide, toluenesulfonyl (Ts) or methylsulfonyl (Ms).

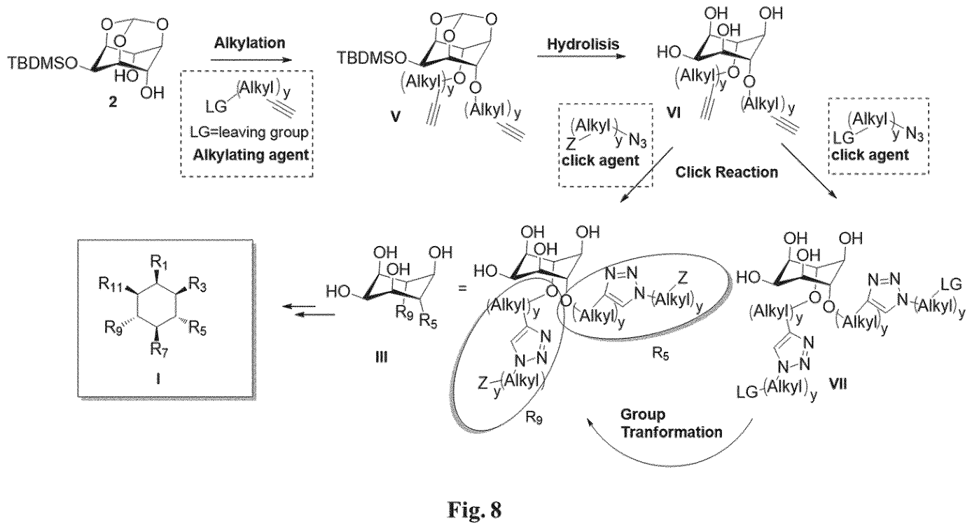

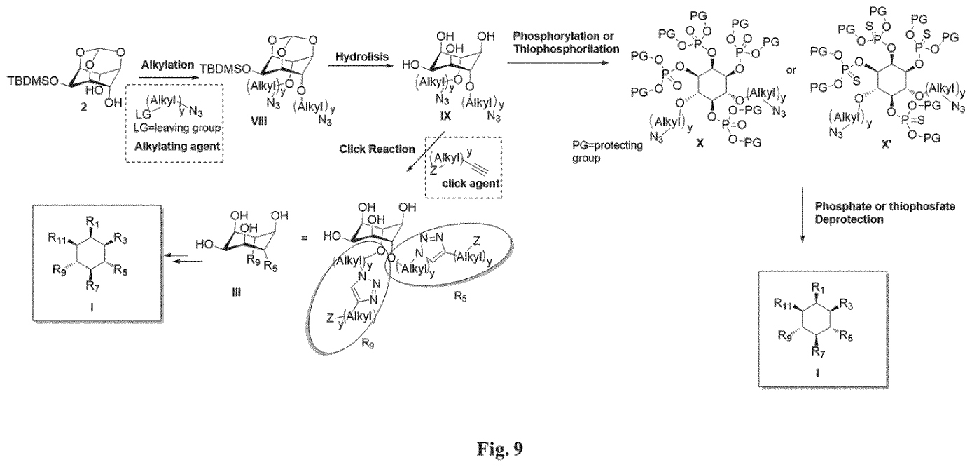

Scheme 2: As an alternative to Scheme 1, e.g., when R₅ and R₉ contain a substituted 1,2,3-triazole, compounds of formula I (i.e., the IP4-4,6 substituted derivatives of the present invention) can be obtained by following the alternative route described in Scheme 2 (FIG. 8). In this way, intermediates of formula III (e.g., Intermediates III-20, III-26, and III-27) can be obtained, e.g., via a click reaction by using as starting materials either an intermediate of formula VI (e.g., Intermediate VI-1) and an alkyl agent. Intermediates VI (e.g., Intermediate VI-1) can be achieved by alkylation and hydrolysis of intermediate 2 with an appropriate reagent.

Scheme 3: As another alternative to Scheme 1 and Scheme 2, e.g., when R₅ and R₉ contain a substituted 1,2,3-triazole, compounds of formula I (i.e., the IP4-4,6 substituted derivatives of the present invention) can be obtained by an alternative route described in Scheme 3 (FIG. 9). In this case, Intermediates of formula III (e.g., Intermediates III-21 to III-25, III-42, and III-43) can be obtained, e.g., via a click reaction by using as starting materials an Intermediate of formula IX (e.g., Intermediates IX-1 to IX5) and an alkynyl click agent. Intermediate IX can be achieved by alkylation and hydrolysis of Compound 2 with an azide alkylating agent. As another alternative to Scheme 1, when R₅ and Ry contain an amine group, compounds of formula I (i.e., the IP4-4,6 substituted derivatives of the present invention) can be obtained as shown in Scheme 3 (FIG. 9). Thus, Intermediates IX (e.g., Intermediates IX-1 to IX-5) can be phosphorylated to obtain Intermediates X (e.g., Intermediates X-1 to X3) and then Intermediates X can be deprotected to obtain compounds of formula I.

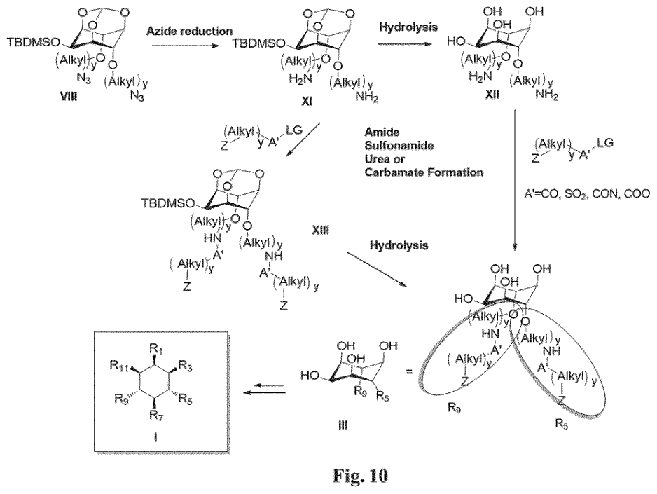

Scheme 4: As an additional alternative to Scheme 1, when R₅ and Ry contain a substituted amide, sulfonamide, carbamate or urea, intermediates III (e.g., Intermediates III-28, III-36 to III-41, and III-44 to III-46) can be obtained as shown in FIG. 10. Thus, intermediates III (e.g., Intermediates III-28, III-36 to III-41, and III-44 to III-46) can be achieved from intermediate VIII (e.g., Intermediates VIII-1 to VIII-5) via an azide reduction (e.g., Intermediates XI-1 to XI-2), subsequent deprotection (e.g., Intermediate XII-1) and, finally, the formation of an amide/sulfonamide/urea or carbamate by reaction of the intermediate XII (e.g., Intermediate XII-1) with an appropriate reagent. Alternatively, the order of the reaction can be changed, first obtaining an amide/sulfonamide/urea or carbamate from intermediate XIII (e.g., Intermediate XIII-1 to XIII-3) and then, by hydrolysis, attaining intermediate III.

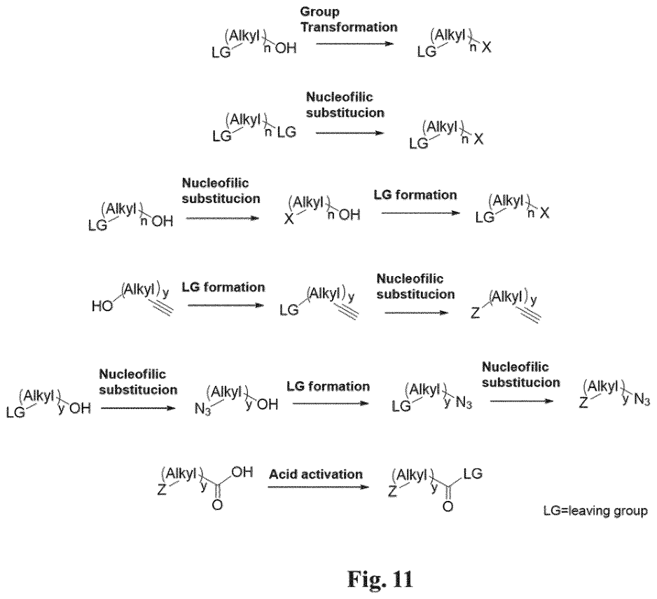

Any alkylating or click agent (included azide or alkynyl) as well as reagents use for amide, sulfonamide, urea or carbamate formation can be commercially available or may be obtained following reactions well known in the field of organic chemistry. In some aspects, methods include, but are not limited to, the general procedures shown in the Scheme 5 (FIG. 11).

Representative IP4-4,6 substituted derivatives of the present invention (e.g., a compound selected from the group consisting of Compound 1 to Compound 53) are presented herein and all of them are in the myo conformation. However, it is to be understood that any exemplary IP4-4,6 substituted derivative of the present invention in the myo conformation is not limited to the representative conformation displayed. Thus, for example, Compounds 1 to 53 and the intermediates presented herein would also encompass the corresponding equivalents in the scyllo, muco, 1D-chiro, 1L-chiro, neo, allo, epi, and cis conformations. In its most stable conformation, the myo-inositol isomer assumes the chair conformation, which moves the maximum number of hydroxyls to the equatorial position, where they are farthest apart from each other. In this conformation, the natural myo isomer has a structure in which five of the six hydroxyls (the first, third, fourth, fifth, and sixth) are equatorial, whereas the second hydroxyl group is axial.

The present invention also provides methods to manufacture a medicament for the treatment of pathological crystallization comprising using an intermediate compound selected from the group consisting of the compounds listed in Table 1. Also provided is a compound of formula I (e.g., selected from the group consisting of Compound 1 to Compound 53) for use as a medicament. Also provided is the use of a compound of formula I (e.g., selected from the group consisting of Compound 1 to Compound 53) for the manufacture of a medicament for the prevention or treatment of a disease related to pathological crystallization.

### III. Pharmaceutical Compositions

The present invention also provides pharmaceutical for use in the treatment, inhibition of progression, and prevention of (a) ectopic calcification or the consequences thereof and (b) a disease and/or condition related to ectopic calcification and the consequences thereof in a subject in need thereof, wherein the pharmaceutical composition comprises at least one IP4-4,6 substituted derivative of the present invention (e.g., a compound selected from the group consisting of Compound 1 to Compound 53). In some aspects, the pharmaceutical composition comprises an IP4-4,6 substituted derivative of the present invention (e.g., a compound selected from the group consisting of Compound 1 to Compound 53) alone or together with one or more pharmaceutically acceptable excipients or carriers.

The term “excipient” as used herein refers to a substance which helps absorption of the elements of the pharmaceutical composition, stabilizes said elements, activates or helps preparation of the composition. Thus, examples of excipients used in parenteral formulations include, but are not limited to, antimicrobial agents (e.g., benzalkonium chloride, metacresol, thimerosal), co-solvents (e.g., ethanol), buffers, tonicity agents (e.g., NaCl) and pH adjusting factors (e.g., carbonate, citrate, phosphate solutions).

As is the case for the excipient, the “pharmaceutically acceptable vehicle” is a substance used in the composition to dilute any of the components contained therein to a determined volume or weight (e.g., a 0.9% (w/v) NaCl aqueous solution). The pharmaceutically acceptable vehicle is an inert substance or a substance with an analogous action to any of the elements comprising the pharmaceutical composition of the present invention. The role of said vehicle is to allow the incorporation of other elements, allow better dosing and administration or to provide consistency and shape to the composition.

Pharmaceutical compositions can comprise from approximately 1% to approximately 95% active ingredient. In some aspects, e.g., the pharmaceutical compositions of the present invention can comprise from approximately 20% to approximately 90% active ingredient (i.e., an IP4-4,6 substituted derivative of the present invention or a combination thereof, alone or in combination, e.g., with one or more additional therapeutic agents).

Formulations of a pharmaceutical composition suitable for parenteral administration comprise the active ingredient, e.g., an IP4-4,6 substituted derivative of the present invention (e.g., a compound selected from the group consisting of Compound 1 to Compound 53), combined with a pharmaceutically acceptable carrier, such as sterile water or sterile isotonic saline (e.g., a 0.9% (w/v) NaCl aqueous solution). Such formulations can be prepared, packaged, or sold in a form suitable for bolus administration or non-bolus administration. Injectable formulations can be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations can further comprise one or more additional ingredients including, but not limited to, suspending, stabilizing, or dispersing agents.

In some aspects, in a formulation for parenteral administration, the active ingredient, e.g., an IP4-4,6 substituted derivative of the present invention (e.g., a compound selected from the group consisting of Compound 1 to Compound 53), is provided in dry (i.e., powder or granular) form for reconstitution with a suitable vehicle (e.g., sterile pyrogen-free water) prior to parenteral administration of the reconstituted composition.

The pharmaceutical compositions can be prepared, packaged, or sold in the form of a sterile injectable aqueous or oily suspension or solution. This suspension or solution can be formulated according to the known art, and may comprise, in addition to the active ingredient (e.g., an inositol phosphate of the present invention), additional ingredients such as the dispersing agents, wetting agents, or suspending agents described herein. Such sterile injectable formulations can be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides.

Other administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt. Compositions and methods of making formulations for administering the IP4-4,6 substituted derivatives of the present invention, including controlled- or sustained-release formulations containing the said active agents, are described in the art. See, e.g., Remington: The Science and Practice of Pharmacy, 23^{rd} Ed., 2021, Academic Press; U.S. Pat. Nos. 6,340,475, 6,488,962, 6,451,808, 5,972,389, 5,582,837, and 5,007,790; US20030147952, US20030104062, US20030104053, US20030044466, US20030039688, and US20020051820; WO2003035041, WO2003035040, WO2003035029, WO2003035177, WO2003035039, WO2002096404, WO2002032416, WO2001097783, WO2001056544, WO2001032217, WO1998055107, WO1998011879, WO1997047285, WO1993018755, and WO1990011757.

Medicaments according to the invention are manufactured by methods known in the art, especially by conventional mixing, coating, granulating, dissolving or lyophilizing.

The present invention also provides a compound or a combination of compounds or pharmaceutical formulation according to any of the above aspects of the invention, in the broadest definition given, or as specified in any of the aspects presented above, for use as a medicament.

The present invention also provides a compound or combination of compounds or pharmaceutical formulation according to any of the above aspects of the invention, in the broadest definition given, or as specified in any of the aspects presented above, for use in the treatment and/or prevention of a disease or condition disclosed herein.

The present invention also provides a compound or combination of compounds or pharmaceutical formulation according to any of the above aspects of the invention, in the broadest definition given, or as specified in any of the aspects presented above, for the manufacture of a medicament for the prevention and/or treatment of a disease or condition disclosed herein.

### IV. Articles of Manufacture and Kits

The present invention also provides articles of manufacture and kits. Such articles of manufacture and kits can comprise a container (e.g., a box) comprising one or more vials containing a formulation comprising (a) one or more of the IP4-4,6 substituted derivatives of the present invention (e.g., a compound selected from the group consisting of Compound 1 to Compound 53) (b) one or more pharmaceutical compositions of the invention and/or (c) solvents for their medical administration or other uses according to the methods disclosed herein.

A kit or article of manufacture provided according to this invention can also comprise brochures or instructions describing the process of medical administration and dosages disclosed herein, or the use of the IP4-4,6 substituted derivatives of the present invention (e.g., a compound selected from the group consisting of Compound 1 to Compound 53) according to the methods disclosed herein. In some aspects, kit or article of manufacture can comprise multiple vials, each one of them containing a single dose. In other aspects, kit or article of manufacture can comprise one or more vials, each one of them comprising more than one dose.

In some aspects, the article of manufacture is a bag containing a solution of an IP4-4,6 substituted derivative of the present invention (e.g., a compound selected from the group consisting of Compound 1 to Compound 53). In other aspects, the article of manufacture is a bottle (e.g., a glass bottle or a plastic bottle) containing a solution of an IP4-4,6 substituted derivative of the present invention (e.g., a compound selected from the group consisting of Compound 1 to Compound 53). In some aspects, the article of manufacture is a bag containing an IP4-4,6 substituted derivative of the present invention (e.g., a compound selected from the group consisting of Compound 1 to Compound 53) in powder form for reconstitution in an appropriate solvent. In other aspects, the article of manufacture is a bottle (e.g., a glass bottle or a plastic bottle) containing an IP4-4,6 substituted derivative of the present invention (e.g., a compound selected from the group consisting of Compound 1 to Compound 53) in powder form for reconstitution in an appropriate solvent.

The kits and articles of manufacture can include instructions for carrying out one or more administrations of the IP4-4,6 substituted derivative of the present invention (e.g., a compound selected from the group consisting of Compound 1 to Compound 53) according to the methods and dosages disclosed herein.

Instructions included in the kits and articles of manufacture can be affixed to packaging material or can be included as a package insert. While the instructions are typically written or printed materials, they are not limited to such. Any medium capable of storing such instructions and communicating them to an end user is contemplated. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### V. Uses of IP4-4,6 Substituted Derivatives of Formula I

In one aspect, the present invention refers to IP4-4,6 substituted derivative compounds of general formula I (e.g., a compound selected from the group consisting of Compound 1 to Compound 53) for use in the treatment, inhibition of progression, and prevention of ectopic calcification or the consequences thereof in a subject in need thereof. In some aspects, the present invention relates to a method for the treatment, inhibition of progression, and prevention of ectopic calcification or the consequences thereof in a subject in need thereof, which comprises administering a therapeutically effective amount of the IP4-4,6 substituted derivative compound of the invention. In some aspects, the invention refers to the use of an IP4-4,6 substituted derivative compound of the invention for the manufacture of a medicament for the treatment, inhibition of progression, and prevention of ectopic calcification or the consequences thereof in a subject in need thereof. In some aspects, the IP4-4,6 substituted derivative is a compound of general formulas II to XIV or any combination thereof. In some aspects, the IP4-4,6 substituted derivative is Compound 1, Compound 3, Compound 6, Compound 27 or any combination thereof. In some aspects, a therapeutically effective amount of the IP4-4,6 substituted derivative of the invention is administered to the subject in need thereof.

In some aspects, the IP4-4,6 substituted derivatives of the present invention (e.g., a compound selected from the group consisting of Compound 1 to Compound 53) can be administered by the topical, enteral or parenteral route of administrations. In some aspects, the parenteral administration is via the intravenous, intraperitoneal, intramuscular, intraarterial or subcutaneous route of administration. In some aspects, the compound can be administered as a component of a hemodialysis, hemofiltration or peritoneal dialysis solution.

In some aspects, the IP4-4,6 substituted derivative of the present invention (e.g., a compound selected from the group consisting of Compound 1 to Compound 53) can be administered by any appropriate method, e.g., a method that provokes a non-bolus type release or effect, such as intravascular (e.g., intravenous) infusion, other parenteral (e.g., subcutaneous, subcutaneous depot, intraperitoneal, intramuscular, intradermal, intrathecal, epidural, spinal or others known to a person skilled in the art), topical (e.g., intranasal, inhalation, intravaginal, transdermal or others known to a person skilled in the art), enteral (e.g. oral, sublingual, rectal) administrations, oral, spinal, intraperitoneal preparations or others known to a person skilled in the art.

In the particular case of patients treated with dialysis, an appropriate method of administration consists of an administration (e.g, a non-bolus type) of an IP4-4,6 substituted derivative of the present invention (e.g., a compound selected from the group consisting of Compound 1 to Compound 53) via a dialysis apparatus (before or after the filter) instead of directly injecting the IP4-4,6 substituted derivative of the present invention into the patient intravenously. Thus, blood can be treated with the IP4-4,6 substituted derivative of the present invention as it leaves the patient and circulates through the dialysis circuit and, when the blood containing the IP4-4,6 substituted derivative of the present invention returns to the body, the IP4-4,6 substituted derivative has been introduced into the blood in a manner that presents a series of advantages. In the case of dialysis patients, administration of an IP4-4,6 substituted derivative of the present invention (e.g., a compound selected from the group consisting of Compound 1 to Compound 53) via the dialysis apparatus allows the blood to equilibrate with the dialysis fluid prior to returning to the body.

In some aspects, an IP4-4,6 substituted derivative of the present invention (e.g., a compound selected from the group consisting of Compound 1 to Compound 53) is administered intravenously via intravenous infusion. In another aspect, an IP4-4,6 substituted derivative of the present invention is administered subcutaneously. In yet another aspect, an IP4-4,6 substituted derivative of the present invention is administered topically. In some aspects, when an IP4-4,6 substituted derivative of the present invention is administered to a patient undergoing dialysis such administration (e.g., intravenous administration via infusion) can occur during a dialysis treatment. In some aspects, the IP4-4,6 substituted derivative of the present invention is administered before a dialysis treatment. In some aspects, the IP4-4,6 substituted derivative of the present invention is administered after a dialysis treatment.

In some aspects, the present invention refers also to a pharmaceutical composition for use in the treatment, inhibition of progression, and prevention of ectopic calcification or the consequences thereof in a subject in need thereof comprising an IP4-4,6 substituted derivative of the present invention or any combination thereof, and at least one pharmaceutically acceptable excipient. In some aspects, the IP4-4,6 substituted derivative is a compound of general formulas II to XIV or any combination thereof. In some aspects, the IP4-4,6 substituted derivative is Compound 1, Compound 3, Compound 6, Compound 27 or any combination thereof. In some aspects, a therapeutically effective amount of the pharmaceutical composition of the invention is administered to the subject in need thereof.

The present invention also provides methods to manufacture a medicament for the treatment, inhibition of progression, and prevention of ectopic calcification or the consequences thereof which comprises using an intermediate compound selected from the group consisting of the compounds listed in Table 1.

Ectopic calcifications (e.g., cutaneous, subcutaneous calcifications) are related to the pathological crystallization of calcium and arise as complications in numerous diseases and conditions. Ectopic calcifications are often damaging in soft tissues such as aorta, brain, carotid, femoral, heart, heart valves, joints, kidney, and lungs. This is often taken for granted as an age-dependent process, but recent data suggest that a myriad of molecules might modulate this process in an active fashion (Nitschke Y, et al., Trends Cardiovasc Med. 2012; 22(6):145-149). The IP4-4,6 substituted derivatives of the present invention are selective calcification inhibitors that work by binding to the growth sites of hydroxyapatite (HAP) crystals, thereby selectively inhibiting the final common step in the pathway of ectopic calcification, including vascular calcification. Since the IP4-4,6 substituted derivatives of the present invention are effective in inhibiting vascular calcification in various types of soft tissue, they could be useful for treating diseases and/or conditions associated to the ectopic calcification of such specific types of soft tissue.

In one aspect, the present invention refers to a IP4-4,6 substituted derivatives compound or pharmaceutical composition of the invention for use in the treatment, inhibition of progression, and prevention of a disease and/or condition related to ectopic calcification or the consequences thereof in a subject in need thereof.

In some aspects, the disease and/or condition related to ectopic calcification or the consequences thereof according to the invention include, but are not limited to, adrenal and intracranial calcification in familial cerebral cavernous malformations, adynamic bone, age-related macular degeneration (AMD) related to calcium deposits, bone cancer, bone mineral disease, breast calcification, calcific band keratopathy, calcific tendinitis, calcification in osteoarthritis, calcification of articular cartilage in osteoarthritis, calcification of joints and arteries (CALJA), calcification of the seminal vesicles, calcinosis cutis, calciphylaxis (CUA), calcium pyrophosphate deposition disease (CPPD), cardiovascular diseases and/or associated conditions, chondrocalcinosis, colon cancer, diabetic kidney disease, dystrophic calcification, failure of renal transplant grafts, familial cerebral cavernous malformations (FCCM), fibrodysplasia ossificans progressiva (FOP), hyperostosis-hyperphosphatemia syndrome (HHS), hyperphosphatemic familial tumoral calcinosis (HFTC), idiopathic brain calcification (Fahr's disease), idiopathic mesenteric phlebosclerosis (IMP), kidney stones (i.e., renal lithiasis), metastatic calcification, nephrocalcinosis, neurocysticercosis-related calcification, osteomalacia, osteoporosis, pineal calcification, phlebosclerotic colitis, podagra, primary familial brain calcification (PFBC), primary hyperoxaluria (PH), pseudoxantoma elasticum (PXE), rheumatoid arthritis, sialolithiasis, Sjorgen's syndrome parotid glands calcification, seminal vesicle calculi, skin cancer, soft tissue calcification owing to sarcoidosis, Wolman's disease adrenal calcification, and wound healing related to diabetic ulcers.

In some aspects, the cardiovascular diseases and/or associated conditions related to ectopic calcification or the consequences thereof according to the invention include, but are not limited to acute ischemic stroke (ACS), aneurysm, angina pectoris (chronic stable angina), aortic artery calcification, aortic calcification, aortic stenosis, aortic valve calcification, arrhythmia, arteriosclerosis, arterial stiffness, arteriovenous fistula (AVF) failure, atherosclerosis, calcific aortic valve stenosis (CAVS), cardiac death, cardiac disease, cardiovascular calcification, cardiovascular disease in chronic kidney disease (CKD) patients, cardiovascular disease linked to aging, cardiovascular mortality, cerebrovascular disease, congestive heart failure, coral reef aorta (CRA), coronary artery calcification, coronary artery disease, coronary disease, critical limb ischemia (CLI), electrocardiogramormalities, general arterial calcification of infancy (GACI), heart failure, hypertension, ischemia, left ventricular hypertrophy, major adverse cardiovascular events (MACE) in hemodialysis (HD) patients, Mönckeberg's medial sclerosis (MMS), myocardial calcification, myocardial infarction, myocardial ischemia, pericardial calcification, peripheral arterial disease (PAD), peripheral vascular disease (PVD), porcelain aorta and calcification of anastomosis site after coronary artery bypass grafting (CABG), portal vein calcification, stroke, thrombosis, valvular calcification, and vascular calcification.

In some aspects, the present invention refers to IP4-4,6 substituted derivative compounds of general formula I (e.g., a compound selected from the group consisting of Compound 1 to Compound 53) or the pharmaceutical compositions of the invention for use in the treatment, inhibition of progression, and prevention of ectopic calcification or the consequences thereof in a subject in need thereof, wherein the ectopic calcification occurs or may occur in aorta, brain, carotid, femoral, heart, heart valve, joint, kidney or lung tissue or any combination thereof. Examples of diseases and/or conditions related to ectopic calcification or the consequences thereof in aorta tissue include, but are not limited to, aortic artery calcification, aortic calcification, aortic stenosis, aortic valve calcification, arteriosclerosis, arterial stiffness, atherosclerosis, calcific aortic valve stenosis (CAVS), cardiovascular disease linked to aging, cardiovascular mortality, coral reef aorta (CRA), electrocardiogramormalities, general arterial calcification of infancy (GACI), heart failure, peripheral arterial disease (PAD), and peripheral vascular disease (PVD). Examples of diseases and/or conditions related to ectopic calcification or the consequences thereof in brain tissue include, but are not limited to, adrenal and intracranial calcification in familial cerebral cavernous malformations, cerebrovascular disease, familial cerebral cavernous malformations (FCCM), idiopathic brain calcification (Fahr's disease), pineal calcification, and primary familial brain calcification (PFBC). Examples of diseases and/or conditions related to ectopic calcification or the consequences thereof in carotid tissue include, but are not limited to ischemia, peripheral arterial disease (PAD), peripheral vascular disease (PVD), stroke, and thrombosis. Examples of diseases and/or conditions related to ectopic calcification or the consequences thereof in femoral tissue include, but are not limited to, arteriosclerosis, arterial stiffness, atherosclerosis, cardiovascular calcification, cardiovascular disease in chronic kidney disease (CKD) patients, cardiovascular disease linked to aging, cardiovascular mortality, critical limb ischemia (CLI), general arterial calcification of infancy (GACI), ischemia, peripheral arterial disease (PAD), peripheral vascular disease (PVD), thrombosis, and vascular calcification. Examples of diseases and/or conditions related to ectopic calcification or the consequences thereof in heart and heart valve tissues include, but are not limited to, aneurysm, angina pectoris (chronic stable angina), aortic valve calcification, arrhythmia, arteriosclerosis, arterial stiffness, atherosclerosis, calcific aortic valve stenosis (CAVS), cardiac death, cardiac disease, cardiovascular calcification, cardiovascular disease in chronic kidney disease (CKD) patients, cardiovascular disease linked to aging, cardiovascular mortality, cerebrovascular disease, congestive heart failure, coronary artery calcification, coronary artery disease, coronary disease, electrocardiogramormalities, general arterial calcification of infancy (GACI), heart failure, hypertension, ischemia, left ventricular hypertrophy, myocardial infarction, and myocardial ischemia. Examples of diseases and/or conditions related to ectopic calcification or the consequences thereof in joint tissue include, but are not limited to, calcific tendinitis, calcification in osteoarthritis, calcification of articular cartilage in osteoarthritis, calcification of joints and arteries (CALJA), dystrophic calcification, ad pseudogout. Examples of diseases and/or conditions related to ectopic calcification or the consequences thereof in kidney tissue include, but are not limited to, failure of renal transplant grafts, kidney stones (i.e., renal lithiasis), nephrocalcinosis, and primary hyperoxaluria (PH). Examples of diseases and/or conditions related to ectopic calcification or the consequences thereof in lung tissue include, but are not limited to, dystrophic calcification, and metastatic calcification. Examples of diseases and/or conditions related to ectopic calcification or the consequences thereof in skin tissue include, but are not limited to, calciphylaxis (CUA) and Pseudoxanthoma elasticum (PXE). In some aspects, the IP4-4,6 substituted derivative is a compound of general formulas II to XIV or any combination thereof. In some aspects, the IP4-4,6 substituted derivative is Compound 1, Compound 3, Compound 6, Compound 27 or any combination thereof.

As indicated above, the administration of the IP4-4,6 substituted derivatives of the present invention to subject inhibits the formation and/or growth of hydroxyapatite (HAP) crystals and their deposition in ectopic calcifications. This mechanism of action could mediate the therapy of many diseases and/conditions related to ectopic calcification and the consequences therefor. For instance, as HAP formation is inhibited, arterial stiffening would be reduced, thus improving the perfusion of coronary arteries during diastole and relieving any associated symptoms of ischemia, such as angina pectoris. Similarly, the IP4-4,6 substituted derivatives of the present invention may also be effective in reducing the risk of cardiovascular events in patients. By inhibiting HAP formation, the IP4-4,6 substituted derivatives of the present invention would slow down the progression of coronary artery calcification (CAC). Since CAC is associated with increased cardiovascular mortality, a reduction in its rate of progression may reduce the risk of cardiovascular events. Moreover, the IP4-4,6 substituted derivatives of the present invention may additionally slow down the calcification of aortic valve leaflets, in view of their inhibitory properties over HAP formation. Pseudoxanthoma elasticum (PXE) is characterized by ectopic mineralization and fragmentation of elastic fibers in the skin, eye, vascular, and gastrointestinal system. As the IP4-4,6 substituted derivatives of the present invention inhibit HAP formation, the eyesight of PXE patients, for example, could be stabilized or improved.

In some aspects, the present invention refers to IP4-4,6 substituted derivative compounds of general formula I (e.g., a compound selected from the group consisting of Compound 1 to Compound 53) or the pharmaceutical compositions of the invention for use in the treatment, inhibition of progression, and prevention of a disease and/or condition related to ectopic calcification or the consequences thereof in a subject in need thereof, wherein (i) a therapeutically effective amount of the IP4-4,6 substituted derivative or pharmaceutical composition of the invention is administered to the subject and (ii) the disease and/or condition is selected from the group consisting of angina pectoris (chronic stable angina), calcific aortic valve stenosis, calciphylaxis, cardiovascular disease in CKD patients, peripheral arterial disease, critical limb ischemia, general arterial calcification of infancy, pseudogout, primary hyperoxaluria, and Pseudoxanthoma elasticum. In some aspects, the IP4-4,6 substituted derivative is a compound of general formulas II to XIV or any combination thereof. In some aspects, the IP4-4,6 substituted derivative is Compound 1, Compound 3, Compound 6, Compound 27 or any combination thereof.

In some aspects, the present invention relates to a method for the treatment, inhibition of progression, and prevention of ectopic calcification or the consequences thereof in a subject in need thereof, which comprises administering a therapeutically effective amount of the IP4-4,6 substituted derivative or pharmaceutical composition of the invention. In some aspects, an IP4-4,6 substituted derivatives of formulas II and VII, or any combination thereof, is administered to the subject. In some further aspects, Compound 1, Compound 3, Compound 6, and Compound 27 or any combination thereof, is administered to the subject.

In some aspects, the consequence of the ectopic calcification is, e.g., (i) a functional complication, (ii) pain, (iii) a trophic complication, (iv) an infection, or (v) a combination thereof. In some aspects, the function complication is, e.g., a limitation of range of motion and/or joint function. In some aspects, the trophic complication is, e.g., ischemia and/or a lesion. In some aspects, the lesion is, e.g., necrosis of the cutaneous and/or subcutaneous tissues.

In some aspects, administration of the dosage of IP4-4,6 substituted derivative or pharmaceutical composition of the present invention to a subject in need thereof causes a reduction in lesions, e.g., as determined by the Bates-Jensen Wound Assessment tool or other methods known in the art (Bates-Jensen B, Decubitus 1992; 5 (6): 20-28). In some aspects, the reduction in lesions comprises, e.g., a reduction in the severity of the lesions, a reduction in the size of the lesions, and reduction in the duration of the lesions, or a combination thereof. In some aspects, administration of the dosage of IP4-4,6 substituted derivative or pharmaceutical composition of the present invention to the subject causes an improvement in lesion healing. In some aspects, the administration of the IP4-4,6 substituted derivative or pharmaceutical composition of the present invention to the subject causes a reduction in pain. In some aspects, the subject has renal failure. In some aspects, the subject is on hemodialysis. In some aspects, subject is human.

In some aspects, administration of the IP4-4,6 substituted derivative or pharmaceutical composition of the present invention to a subject in need thereof an improvement on global wound quality of life (QoL) as determined by using a validated wound-associated QoL questionnaire or other methods known in the art (Augustin M, et al., Int Wound J. 2017; 14(6):1299-1304). In some aspects, the subject has renal failure. In some aspects, the subject is on hemodialysis. In some aspects, subject is human.

Kidney failure, also known as renal impairment or kidney disease, is a disease that causes a progressive loss of kidney function, with a concomitant decrease in the glomerular filtration rate (GFR) or index. Renal impairment, together with treatment of the disease, leads to hypercalcemia and hyperphosphatemia. Hypercalcemia and hyperphosphatemia may cause cardiovascular calcification, although a deficiency of repressor factors (e.g., matrix Gla protein, osteopontin, fetuin, or vitamin K) or an imbalance in promoting factors (e.g., vitamin D, FGF23, inflammatory cytokines, lipid deposits, apoptotic bodies, nucleational complexes) may delay or accelerate the process. Patients with renal impairment are commonly described as patients with CKD-MBD (chronic kidney disease-mineral bone disease) as altered kidney function provokes a cascade of effects that also affect bone remodeling.

In some aspects, the IP4-4,6 substituted derivatives and pharmaceutical compositions of the present invention can be used for treating, inhibiting the progression, and preventing some of the diseases and/or conditions related to ectopic calcification or the consequences thereof, as listed above, in patients with kidney failure. In some aspects, the disease and/or condition related to ectopic calcification or the consequences thereof is a kidney failure-related disease. In some aspects, the patient with kidney failure is on dialysis. In some aspects, the IP4-4,6 substituted derivatives and pharmaceutical compositions of the invention are administered to the patient with kidney failure via intravenous infusion. In some aspects the intravenous infusion is performed using a dialysis apparatus. In some aspects, the IP4-4,6 substituted derivative is a compound of general formulas II to XIV or any combination thereof. In some aspects, the IP4-4,6 substituted derivative is Compound 1, Compound 3, Compound 6, Compound 27 or any combination thereof. In some aspects, a therapeutically effective amount of the IP4-4,6 substituted derivative of the invention is administered to the patient with kidney failure.

In some aspects, the IP4-4,6 substituted derivatives of the present invention can be used in dialysis liquids, e.g., during hemodialysis. Accordingly, the present invention also provides dialysis liquids, e.g., hemodialysis liquids comprising an IP4-4,6 substituted derivative of the present invention (e.g., a compound selected from the group consisting of Compound 1 to Compound 53), a pharmaceutically acceptable salt thereof, or any combination thereof, wherein the administration of the IP4-4,6 substituted derivative, pharmaceutically acceptable salt thereof, or combination thereof to the subject in need thereof is effective in treating, inhibiting the progression, and preventing (i) ectopic calcification and the consequences thereof or (ii) a disease and/or condition related to ectopic calcification or the consequences thereof.

### A. Synthesis of Protected Myo-Inositol Agents, Alkylating or Click Agents and Activated Acids

### A.1. Synthesis of Protected Myo-Inositol Agents (1) and (2)

1,3,5-O-Methylidyne-myo-inositol (1): The synthesis of (1) was previously described in the literature (Martin S, et al., J. Org. Chem. 1994; 59(17):4805-4820)

2-O-tert-Butyldimethylsilyl-1,3,5-O-methylidyne-myo-inositol (2): The synthesis of (2) was previously described in the literature (Kadirvel M, et al., Org. Biomol. Chem. 2008; 6 (11): 1966-1972).

### A.2. Alkylating or Click Agents' Synthesis

(((10-Bromodecyl)oxy)methyl)benzene (4): The synthesis of (4) was previously described in the literature (Hanbali M., et al., Bioorg. Med. Chem. Letters 2006; 16(10):2637-2640).

(((14-Bromotetradecyl)oxy)methyl)benzene (5): The synthesis of (5) was previously described in the literature (Hitosugi S, et al., Organic Letters 2014; 16(3):844-847).

1-Bromo-7-methoxyheptane (6): A mixture of 1,7-dibromoheptane (3.27 mL, 19.38 mmol) and sodium methoxide 5N (3.88 mL, 19.38 mmol) was stirred for 4 h at 40° C. The reaction mixture was filtered and concentrated in vacuum. The residue was purified by flash chromatography (silica gel, hexane (Hex):ethyl acetate (EtOAc) 10:1) to afford 1.4 g of (6) (34.5%). ¹H NMR (400 MHz, Chloroform-d) δ 3.40 (t, J=6.9 Hz, 2H), 3.36 (t, J=6.6 Hz, 2H), 3.33 (s, 3H), 1.89-1.82 (m, 2H), 1.60-1.53 (m, 2H), 1.47-1.40 (m, 2H), 1.39-1.32 (m, 4H).

### 9-Methoxynonyl 4-methylbenzenesulfonate (7)

Step 1: 9-Methoxynonan-1-ol (3): A mixture of 9-bromononan-1-ol (0.5 g, 2.24 mmol) and sodium methoxide 4N (25 mL, 100 mmol)) was stirred for 18 h at 40° C. The reaction mixture was filtered and concentrated in vacuum. The residue was purified by flash chromatography (silica gel, Hex:EtOAc 4:1) to afford 324 mg of (3) (83% yield). ¹H NMR (400 MHz, Chloroform-d) δ 3.63 (t, J=6.6 Hz, 2H), 3.36 (t, J=6.6 Hz, 2H), 3.33 (s, 3H), 1.59-1.52 (m, 4H), 1.29 (m, 10H).

Step 2: 9-Methoxynonyl 4-methylbenzenesulfonate (7): To a solution of (3) (1.5 g, 8.61 mmol) in dichloromethane (DCM, 28.7 mL) triethylamine (TEA (1.80 mL, 12.91 mmol) and tosyl chloride (Ts-Cl, 2.13 g, 11.19 mmol) were added. The reaction mixture was stirred for 24 h at rt then quenched with water and washed with brine. The organic layer was dried over with Na₂SO₄, filtered and concentrated in vacuum. The residue was purified by flash chromatography (silica gel, Hex:EtOAc 4:1) to afford 2.15 g of (7) (76%). ¹H NMR (400 MHz, Chloroform-d) δ 7.78 (d, J=8.3 Hz, 2H), 7.34 (d, J=8.3 Hz, 2H), 4.01 (t, J=6.5 Hz, 2H), 3.35 (t, J=6.6 Hz, 2H), 3.32 (s, 3H), 2.45 (s, 3H), 1.61 (dt, J=8, 6.6 Hz, 2H), 1.56-1.51 (m, 2H), 1.32-1.22 (m, 10H).

3-Azidopropyl 4-methylbenzenesulfonate (8): To a solution of 3-azidopropan-1-ol (1 g, 9.89 mmol) in DCM (0.2M) at 0° C., TEA (2.07 mL, 14.84 mmol) and Ts-Cl (2.26 g, 11.87 mmol) were added. The reaction mixture was stirred for 24 h at rt then quenched with water and washed with brine. The organic layer was dried over with Na₂SO₄, filtered and concentrated in vacuum. The residue was purified by flash chromatography (silica gel, Hex:EtOAc 4:1) to afford 1.27 g of (8) (50%). ¹H NMR (400 MHz, Chloroform-d) δ 7.80 (d, J=8.3 Hz, 2H), 7.36 (dd, J=8.3, 0.7 Hz, 2H), 4.11 (t, J=6.3 Hz, 2H), 3.38 (t, J=6.3 Hz, 2H), 2.46 (s, 3H), 1.89 (p, J=6.3 Hz, 2H).

### 5-Azidopentyl 4-methylbenzenesulfonate (10)

Step 1: 5-Azidopentan-1-ol (9): A solution of 5-bromopentan-1-ol (0.10 mL, 0.89 mmol) and sodium azide (63 mg, 0.98 mmol) in dimethylformamide (DMF, 0.2 M) was stirred for 18 h at 80° C. Then, the reaction mixture was quenched with water/EtOAc and was washed with brine (3×). The organic layer was dried over with Na₂SO₄, filtered, and concentrated in vacuum to afford 57 mg of (9) (50%). ¹H NMR (400 MHz, Chloroform-d) δ 3.63 (t, J=6.8 Hz, 2H), 3.26 (t, J=6.8 Hz, 2H), 1.61 (p, J=7.2 Hz, 2H), 1.58 (p, J=6.8 Hz, 2H), 1.52-1.36 (m, 2H).

Step 2: 5-Azidopentyl 4-methylbenzenesulfonate (10): To a solution of (9) (54 mg, 0.42 mmol) in DCM (0.2 M) at 0° C., TEA (87 μL, 0.63 mmol) and Ts-Cl (80 mg, 0.42 mmol) were added. The reaction mixture was stirred for 60 h at rt, and then quenched with water/DCM and washed with brine. The organic layer was dried over with Na₂SO₄, filtered and concentrated in vacuum to afford 92 mg of (10) (78%). HPLC-MS (Condition A): rt=3.00 min; m/z: 284 [M+1]⁺.

### 10-Azidodecyl 4-methylbenzenesulfonate (12)

Step 1: 10-Azidodecan-1-ol (11): A solution of 10-bromodecan-1-ol (0.42 mL, 2.11 mmol) and sodium azide (151 mg, 2.32 mmol) in DMF (0.2 M) was stirred for 18 h at 80° C. Then, reaction mixture was quenched with water/EtOAc and washed with brine (3×). The organic layer was dried over with Na₂SO₄, filtered and concentrated in vacuum to afford 388 mg of (11) (92%). ¹H NMR (400 MHz, Chloroform-d) δ 3.64 (t, J=6.8 Hz, 2H), 3.25 (t, J=6.8 Hz, 2H), 1.59 (p, J=6.8 Hz, 2H), 1.56 (p, J=6.8 Hz, 2H), 1.44-1.19 (m, 12H).

Step 2: 10-Azidodecyl 4-methylbenzenesulfonate (12): To a solution of (11) (388 mg, 1.95 mmol) in DCM (0.2M) at 0° C., TEA (407 μL, 2.92 mmol) and Ts-Cl (371 mg, 1.95 mmol) were added. The reaction mixture was stirred for 60 h at rt then, quenched with water/DCM and washed with brine. The organic layer was dried over with Na₂SO₄, filtered, and concentrated in vacuum. The residue was purified by flash chromatography (silica gel Hex:EtOAc 1:1) to afford 234 mg of (12) (34%). ¹H NMR (400 MHz, Chloroform-d) δ 7.79 (d, J=8.1 Hz, 2H), 7.34 (d, J=8.1 Hz, 2H), 4.02 (t, J=6.4 Hz, 2H), 3.25 (t, J=6.8 Hz, 2H), 2.45 (s, 3H), 1.63 (p, J=6.4 Hz, 2H), 1.59 (p, =6.8 Hz, 2H), 1.35-1.15 (m, 12H).

1-(5-Bromopentyl)-1H-pyrazole (13): To a mixture of 1H-pyrazole (0.78 g, 11.49 mmol) and Cs₂CO₃ (3.74 mg, 11.49 mmol) in ACN (57 mL) 1,5-dibromopentane (1.56 mL, 11.49 mmol) was added. The mixture was stirred for 18 h at rt. The reaction mixture was filtered and concentrated in vacuum. The residue was purified by flash chromatography (silica gel, Hex:EtOAc 1:1) to afford 1.1 g of (13) (45%). HPLC-MS (Condition A): rt=3.00 min; m/z: 218, 220 [M+1, M+3]⁺.

1-(5-Bromopentyl)-1H-1,2,4-triazole (14): To a mixture of 1H-1,2,4-triazole (0.79 g, 11.50 mmol) and Cs₂CO₃ (3.74 mg, 11.50 mmol) in ACN (57 mL) 1,5-dibromopentane (1.56 mL, 11.49 mmol) was added. The mixture was stirred for 18 h at rt. The reaction mixture was filtered and concentrated in vacuum. The residue was purified by flash chromatography silica gel, Hexane:EtOAc (1:5) to afford 0.55 g of (14) (22%). HPLC-MS (Condition A): rt=2.95 min; m/z: 218, 220 [M, M+2]⁺.

Benzyl 3-azidopropanoate (15): To a solution 3-azidopropanoic acid (1.62 mL, 17.38 mmol) in DMF (1 M), (bromomethyl)benzene (4.1 mL, 34.8 mmol) followed by drop wise addition of TEA (7.3 mL, 52.1 mmol) were added. After stirring for 1 day at rt, the mixture was concentrated, diluted with EtOAc, washed with Na₂CO₃ (1×), water (3×), and brine (1×), dried over with Na₂SO₄, and filtered. The solvents were removed in vacuo to afford 2.90 g of (15) (81%). ¹H NMR (400 MHz, Chloroform-d) δ 7.46-7.29 (m, 5H), 5.17 (s, 2H), 3.60 (t, J=6.5 Hz, 2H), 2.63 (t, J=6.5 Hz, 2H).

### 6-Azidohexyl 4-methylbenzenesulfonate (17)

Step 1: 6-Azidohexan-1-ol (16): A solution of 6-bromohexan-1-ol (428 μL, 3.27 mmol) and sodium azide (850 mg, 13.08 mmol) in DMF (0.8 M) was stirred for 18 h at 80° C. Then, the reaction mixture was quenched with water/EtOAc, and washed with brine (3×). The organic layer was dried over with Na₂SO₄, filtered, and concentrated in vacuum to afford 470 mg of (16) (>99%). ¹H NMR (400 MHz, Chloroform-d) δ 3.65 (t, J=6.8 Hz, 2H), 3.27 (t, J=6.8 Hz, 2H), 1.70-1.56 (m, 4H), 1.40 (m, 4H).

Step 2: 6-Azidohexyl 4-methylbenzenesulfonate (17): At 0° C., p-Ts-Cl (720 mg, 3.78 mmol) was added to a solution of (16) (515 mg, 3.60 mmol) and TEA (1.5 mL, 10.79 mmol) in dry DCM (0.6 M). The reaction mixture was stirred overnight at rt. It was diluted with EtOAc and washed with 10% aqueous solution of NaHSO₄. The aqueous phase was extracted with EtOAc (3×). The combined layers were washed with saturated aqueous solution of NaHCO₃ and dried over anhydrous Na₂SO₄. The solvent was removed in vacuo and the residue was purified by flash chromatography (silica gel Hex:EtOAc 6:1) giving 666 mg of (17) (62%). ¹H NMR (400 MHz, Chloroform-d) δ 7.79 (d, J=8.0 Hz, 2H), 7.35 (d, J=8.0 Hz, 2H), 4.03 (t, J=6.4 Hz, 2H), 3.23 (t, J=6.8 Hz, 2H), 2.45 (s, 3H), 1.66 (p, J=6.4 Hz, 2H), 1.55 (p, J=6.4 Hz, 2H), 1.41-1.18 (m, 4H).

### 4-Azidobutyl 4-methylbenzenesulfonate (19)

Step 1: 4-Azidobutan-1-ol (18): A solution of 4-bromobutan-1-ol (298 μL, 3.27 mmol) and sodium azide (850 mg, 13.08 mmol) in DMF (0.8 M) was stirred for 18 h at 80° C. Then, reaction mixture was quenched with water/EtOAc and was washed with Brine (3×). The organic layer was dried over with Na₂SO₄, filtered, and concentrated in vacuo to afford 213 mg of (18) (56%). ¹H NMR (400 MHz, Chloroform-d) δ 3.69 (t, J=5.9 Hz, 2H), 3.33 (t, J=6.4 Hz, 2H), 1.76-1.59 (m, 4H).

Step 2: 4-Azidobutyl 4-methylbenzenesulfonate (19): At 0° C., Ts-Cl (370 mg, 1.94 mmol) was added to a solution of (18) (213 mg, 1.85 mmol) and TEA (774 μL, 5.55 mmol) in dry DCM (0.6 M). The reaction mixture was stirred overnight at rt. It was diluted with EtOAc and washed with 10% aqueous solution of NaHCO₃. The aqueous phase was extracted with EtOAc (3×). The combined layers were dried over with anhydrous Na₂SO₄. The solvent was removed in vacuo and the residue was purified by flash chromatography (silica gel, Hex:EtOAc 6:1) giving 271 mg of (19) (54%). ¹H NMR (400 MHz, Chloroform-d) δ 7.79 (d, J=8 Hz, 2H), 7.36 (d, J=8 Hz, 2H), 4.06 (td, J=6.1, 0.8 Hz, 2H), 3.26 (t, J=6.6 Hz, 2H), 2.46 (s, 3H), 1.81-1.68 (m, 2H), 1.68-1.60 (m, 2H).

1-(4-(2-Chloroethyl) piperazin-1-yl) ethanone (20): To a solution of 1-(4-(2-hydroxyethyl) piperazin-1-yl) ethanone (1.74 g, 10.10 mmol) in DCM (0.2M) at 0° C., TEA (2.1 mL, 15.15 mmol) and Ts-Cl (2.31 g, 12.12 mmol) were added. The reaction mixture was stirred for 72 h at rt and concentrated in vacuo. The residue was purified by flash chromatography (silica gel, DCM: MeOH 95:5) giving 1.28 g of (20) (66%). HPLC-MS (Condition A): rt=0.24 min; m/z: 191, 193 [M, M+2]⁺.

2-Cyclopropylethyl 4-methylbenzenesulfonate (21): To a solution of 2-cyclopropylethanol (3 mL, 34.0 mmol) in DCM (85 mL), TEA (7.10 mL, 50.9 mmol) and Ts-Cl (7.77 g, 40.8 mmol) were added, and the obtained mixture was stirred for 18 h. Water was added to the reaction mixture and extracted with DCM (2×). The combined organic layer was washed with brine, dried over Na₂SO₄, and concentrated in vacuo. The residue was purified by column chromatography on silica gel (Hex-EtOAc, 4:1) yielding 6.23 g (76%) of (21). ¹H NMR (400 MHz, Chloroform-d) δ 7.78 (d, J=8.2 Hz, 2H), 7.32 (d, J=8.2 Hz, 2H), 4.06 (t, J=6.8 Hz, 2H), 2.43 (s, 3H), 1.51 (q, J=6.8 Hz, 2H), 0.63 (m, 1H), 0.38 (ddd, J=8, 6, 4 Hz, 2H), -0.01 (dt, J=6, 4 Hz, 2H).

2-Cyclopentylethyl 4-methylbenzenesulfonate (22): To a solution of 2-cyclopentylethanol (1.09 mL, 8.76 mmol) in DCM (22 mL), TEA (1.83 mL, 13.1 mmol) and Ts-Cl (2.00 g, 10.5 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.53 g (65%) of (22). ¹H NMR (400 MHz, Chloroform-d) δ 7.81 (d, J=8 Hz, 2H), 7.37 (d, J=8 Hz, 2H), 4.06 (t, J=6.7 Hz, 2H), 2.47 (s, 3H), 1.89-1.76 (m, 1H), 1.76-1.43 (m, 8H), 1.10-0.99 (m, 2H).

3-(4-Methoxyphenyl)propyl 4-methylbenzenesulfonate (23): To a solution of 3-(4-methoxyphenyl) propan-1-ol (0.96 mL, 6.02 mmol) in DCM (15 mL), TEA (1.26 mL, 9.02 mmol) and Ts-Cl (1.38 g, 7.22 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.59 g (82%) of (23). ¹H NMR (400 MHz, Chloroform-d) 8 7.81 (d, J=8 Hz, 2H), 7.37 (d, J=8 Hz, 2H), 7.00 (d, J=8.6 Hz, 2H), 6.80 (d, J=8.6 Hz, 2H), 4.04 (t, J=6 Hz, 2H), 3.80 (s, 3H), 2.61 (dd, J=8.2, 6.8 Hz, 2H), 2.48 (s, 3H), 2.03-1.86 (ddt, J=8.2, 6.8, 6 Hz, 2H).

3-(3-(Trifluoromethyl)phenyl)propyl 4-methylbenzenesulfonate (24): To a solution of 3-(3-(trifluoromethyl)phenyl) propan-1-ol (0.91 mL, 4.90 mmol) in DCM (12 mL), TEA (1.02 mL, 7.35 mmol) and Ts-Cl (1.12 g, 5.88 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.53 g (87%) of (24). ¹H NMR (400 MHz, Chloroform-d) δ 7.63 (d, J=8.5 Hz, 2H), 7.28 (d, J=7.8 Hz, 1H), 7.21-7.16 (m. 2H), 7.19 (d, J=8.5 Hz, 2H), 7.12 (J=7.8 Hz, 1H), 3.88 (t, J=6.2 Hz, 2H), 2.56 (dd, 8.0, 7.6 Hz, 2H), 2.29 (s, 3H), 1.81 (ddt, J=8.0, 7.6, 6.2 Hz, 2H).

3-(p-Tolyl)propyl 4-methylbenzenesulfonate (25): To a solution of 3-(p-tolyl) propan-1-ol (1 g, 6.66 mmol) in DCM (17 mL), TEA (1.39 mL, 9.99 mmol) and Ts-Cl (1.52 g, 7.99 mmol) were added, and the obtained mixture was stirred for 18 h. Water was added to the reaction mixture and extracted with DCM (2×). The combined organic layer was washed with brine, dried over with Na₂SO₄, and concentrated in vacuo. The residue was purified by column chromatography on silica gel (Hex-EtOAc, 4:1) yielding 1.85 g (87%) of (25). ¹H NMR (400 MHz, Chloroform-d) δ 7.81 (d, J=8.0 Hz, 2H), 7.37 (d, J=8.0 Hz, 2H), 7.07 (d, J=8.0 Hz, 2H), 6.98 (d, J=8.0 Hz, 2H), 4.05 (t, J=6.4 Hz, 2H), 2.62 (t, J=7.5 Hz, 2H), 2.48 (s, 3H), 2.32 (s, 3H), 1.95 (tt, J=7.5, 6.4 Hz, 2H).

### 19-Methoxynonadec-10-yn-1-yl 4-methylbenzenesulfonate (31)

Step 1: 2-((9-Bromononyl)oxy)tetrahydro-2H-pyran (26): To a solution of 9-bromononan-1-ol (6.17 g, 27.6 mmol) and 2,3-dihydro-2H-pyran (3.78 mL, 23 mmol), p-TSA, (105 mg, 0.55 mmol) were added. The mixture was stirred for 18 hours at rt. The mixture was diluted with Na₂CO₃ 1N, extracted with ethyl ether (2×), dried over with Na₂SO₄, filtered, and the solvents removed in vacuo. The filtrate was concentrated and purified by column chromatography (0 to 5% EtOAc in Hex) yielding 6.31 g (74%) of (26). ¹H NMR (400 MHz, Chloroform-d) δ 4.56 (dd, J=4.3, 2.7 Hz, 1H), 3.85 (td, J=7.5, 3.8 Hz, 1H), 3.71 (dt, J=9.6, 6.9 Hz, 1H), 3.52-3.32 (m, 4H), 1.88-1.24 (m, 20H).

Step 2: Dec-9-yn-1-yl methanesulfonate (27): To a stirred solution of dec-9-yn-1-ol (3.45 mL, 19.5 mmol) in THF (56 mL), TEA (3.52 mL, 25.3 mmol) and methanesulfonyl chloride (1.97 mL, 25.3 mmol) were added dropwise at 0° C. under a nitrogen atmosphere. The reaction was left stirring for 18 h at rt. The solvent was evaporated; water was added to the reaction mixture and then extracted with DCM. The organic layer was dried over with Na₂SO₄, filtered, and evaporated under reduced pressure to obtain 4.45 g (98%) of (27). HPLC-MS (Condition A): rt=3.58 min.

Step 3: 10-Methoxydec-1-yne (28): To a stirred solution of sodium methoxide 5M in MeOH, (27) (4.45 g, 19.1 mmol) was added and left stirring at 45° C. for 18 h. Then, the suspension was dissolved in water and extracted with Et₂O (2×). The organic layer was dried over with Na₂SO₄ and evaporated to dryness. The crude product was purified by column chromatography (5% EtOAc in Hex) to give (28) (2.58 g, 80%). ¹H NMR (400 MHz, Chloroform-d) δ 3.34 (t, J=5.2 Hz, 2H), 3.31 (s, 3H), 2.16 (td, J=7.2, 2.1 Hz, 2H), 1.91 (t, J=3.2 Hz, 1H), 1.53-1.48 (m, 4H), 1.40-1.27 (m, 8H).

Step 4: 2-((19-Methoxynonadec-10-yn-1-yl)oxy)tetrahydro-2H-pyran (29): To a solution of (28) (4 g, 23.8 mmol) in 30 mL of THF and 25 mL of HMPA cooled at −40° C., n-BuLi (15.5 mL, 24.8 mmol) was slowly added, followed by stirring at the same temperature for 30 minutes, and further stirring at 0° C. for 30 minutes. After cooling to −20° C., a solution of (26) (6.09 g, 19.81 mmol) in 25 mL of HMPA was slowly added. After stirring at the same temperature for 10 minutes, the temperature was increased to rt. Stirring was conducted at the same temperature for 18 hours. Under ice cooling (at 4° C.), an aqueous 10% HCl solution was added, and extracted with tBuMeO (3×). The ethereal layer was washed with saturated brine and further drying over anhydrous MgSO₄. After filtration, the solvent was distilled off under reduced pressure to give a yellow oil. The residue was purified by column chromatography with EtOAc/Hex (1:20), to give (29) (2.36 g, 30%). ¹H NMR (400 MHz, Chloroform-d) δ 4.55 (dd, J=4.4, 2.8 Hz, 1H), 3.86-3.74 (m, 1H) 3.70 (dt, J=9.6, 6.8 Hz, 1H), 3.52-3.46 (m, 1H), 3.39-3.33 (m, 3H), 3.31 (s, 3H), 2.11 (td, J=7.2, 2.1 Hz, 2H), 1.86-1.78 (m, 1H), 1.74-1.65 (m, 1H), 1.53-1.26 (m, 31H), 0.86-0.83 (m, 1H).

Step 4: 19-Methoxynonadec-10-yn-1-ol (30): To a solution of (29) (2.36 g, 6 mmol) in MeOH (10 mL), p-TSA (68 mg, 0.36 mmol) was added portionwise. The resulting solution was stirred at 60° C. for 16 h and then concentrated in vacuo. The residue was dissolved in sat. aq. NaHCO₃ and extracted with tBuMeO (2×). The combined organic extracts were washed with brine solution and dried with Na₂SO₄. No further purification was necessary, yielding 1.32 g (71%) of (30). ¹H NMR (400 MHz, Chloroform-d) δ 3.62 (t, J=6.6 Hz, 2H), 3.34 (t, J=6.7 Hz, 2H), 3.31 (s, 3H), 2.15-2.09 (m, 4H), 1.54 (m, 6H), 1.50-1.40 (m, 5H), 1.40-1.22 (m, 16H).

Step 6: 19-Methoxynonadec-10-yn-1-yl 4-methylbenzenesulfonate (31): To a solution of (30) (1.32 g, 4.25 mmol) in THF (8.5 mL), Ts-Cl (973 mg, 5.10 mmol) and NaOH 5M (1.020 mL, 5.10 mmol) were added and the obtained mixture was stirred for 18 h. Water was added to the reaction mixture and extracted with tBuMeO (2×). The combined organic layer was washed with water, brine, dried over with Na₂SO₄, and concentrated in vacuo. The residue was purified by column chromatography on silica gel (Hex-EtOAc, 6:1) yielding 1.69 g (86%) of (31). ¹H NMR (400 MHz, Chloroform-d) 8 7.65 (d, J=8.2 Hz, 2H), 7.20 (d, J=8.2 Hz, 2H), 3.87 (t, J=6.5 Hz, 2H), 3.22 (t, J=6.7 Hz, 2H), 3.18 (s, 3H), 2.31 (s, 3H), 2.00-1.96 (m, 4H), 1.52-1.45 (m, 2H), 1.44-1.38 (m, 2H), 1.34-1.27 (m, 4H), 1.27-1.05 (m, 18H).

### A.3. Activated Acid Synthesis

Benzyl (2,5-dioxopyrrolidin-1-yl) glutarate (32): To a solution of 5-(benzyloxy)-5-oxopentanoic acid (285 mg, 1.28 mmol) and (2,5-dioxopyrrolidin-1-yl) carbonate (600 mg, 2.34 mmol) in DCM (0.05 M), TEA (357 μL, 2.56 mmol) was added. The reaction mixture was stirred for 24 h at rt, then quenched with water, and washed with brine. The organic layer was dried over with Na₂SO₄, filtered and concentrated in vacuum. The residue was purified by flash chromatography (silica gel, Hex:EtOAc 1:1) to afford 254 mg of (32) (64%). HPLC-MS (Condition B): rt=2.55 min; m/z: 320 [M+1]⁺, 342 [M+23]⁺.

2,5-Dioxopyrrolidin-1-yl thiophene-2-carboxylate (33): To a solution of thiophene-2-carboxylic acid (300 mg, 2.34 mmol) and di(N-succinimidyl) carbonate (600 mg, 2.34 mmol) in DCM (4 mL), TEA (653 μL, 4.68 mmol) was added. The mixture was stirred at rt for 18 h. Water was added to the reaction mixture and extracted with DCM (2×). The combined organic layer was washed with brine, dried over with Na₂SO₄, and concentrated in vacuo. The residue was used without further purification. ¹H NMR (400 MHz, Chloroform-d) δ 8.06 (dd, J=4.8, 0.8 Hz, 1H), 7.80 (dd, J=4.8, 0.8 Hz, 1H), 7.23 (dd, J=4.8 Hz, 1H), 2.88 (br, 4H).

### B. General Procedures

### B.1. Alkylation Procedures

Procedure A: To a solution of (2) (1 eq) in DMF (0.2 M) at 0° C., NaH (2.15 eq) was added. When the addition was complete, the mixture was stirred for 5 minutes at rt. Finally, the alkylating agent was added. The reaction was allowed to stir overnight, then quenched with water, and extracted with EtOAc. The organic layer was dried over with Na₂SO₄, filtered, and concentrated in vacuum. The residue was purified by flash chromatography (silica gel) to afford pure compounds.

Procedure B: To a solution of (2) (1 eq) in DMF (0.2 M) at 0° C., LiH was added. When the addition was complete, the mixture was stirred for 5 minutes at rt. Finally, the alkylating agent was added. The reaction was allowed to stir during different times and temperatures depending on the alkylating agent, then quenched with water, and extracted with EtOAc. The organic layer was dried over with Na₂SO₄, filtered, and concentrated in vacuum. The residue was purified by flash chromatography (silica gel) to afford pure compounds.

### B.2. Hydrolysis Procedures

Procedure C: Intermediate (II), (V), (VIII), (XI) or (XIII) (1 eq) was dissolved in a mixture of methanol/water/DCM/trifluoroacetic acid (3:1:1:1, 0.1 M) and the solution was stirred at rt overnight. Finally, the solvent and excess of TFA was removed under vacuum to afford the desired compounds.

Procedure D: Intermediate (II), (V), (VIII), (XI) or (XIII) (1 eq) was dissolved in a mixture of methanol/water/DCM/trifluoroacetic acid (3:1:1:1, 0.1 M) and the solution was stirred at rt overnight. Then, the solvent and excess of TFA was removed under vacuum. Finally, the residue was dissolved in methanol (MeOH), treated with IRA-400 resin until pH was basic, filtered, and the solvent was removed under vacuum to afford the desired compounds.

Procedure E: To a solution of Intermediate (II), (V), (VIII), (XI) or (XIII) (1 eq) in THF (c=0.1 M), a solution 0.1 M of TBAF in THF (1.2 eq) was added. The reaction mixture was stirred for 18 h at rt, quenched with water, and extracted with EtOAc. The organic layer was dried over with Na₂SO₄, filtered, and concentrated in vacuum. The residue was dissolved in a mixture of MeOH/water/DCM/TFA (3:1:1:1, 0.1 M) and the solution was stirred at rt overnight. Then, the solvent and excess of TFA was removed under vacuum. Finally, the residue was dissolved in methanol (MeOH), treated with IRA-400 resin until pH was basic, filtered, and the solvent was removed under vacuum to afford the desired compounds.

Procedure F: Intermediate (II), (V), (VIII), (XI) or (XIII) (1 eq) was dissolved in a mixture of DCM/TFA (4:1, 0.14 M) and the solution was stirred at rt 1 h. Then, water was added (final concentration 0.1 M) and the reaction mixture was stirred at rt 18 h. Finally, the layers were separated, and the aqueous layer was concentrated in vacuum to afford the desired compound.

Procedure S: Intermediate (II), (V), (VIII), (XI) or (XIII) (1 eq) was dissolved in a mixture of MeOH/HCl 1N (10:1, 0.1 M) and the solution was stirred at 60° C. overnight. Finally, the solvent and excess of HCl were removed under vacuum to afford the desired compounds.

### B.3. Click Reaction Procedures

Procedure K: To a suspension of Intermediate (VI) (1 eq) and CuI (0.03 eq) in DCM (0.1 M), a solution of azide reagent (2 eq), DIPEA (0.07 eq) and AcOH (0.07 eq) in DCM/MeOH (1M) was added. The reaction mixture was stirred at 35° C. for 20 h under inert atmosphere and concentrated in vacuum to afford the desired compound.

Procedure L: To a solution of Intermediate (VI) (1 eq), CuSO_{4·}5H₂O (0.4 eq) and sodium ascorbate (0.6 eq) in water (0.07M), a solution of azide reagent (2 eq) in THF (0.35 M) was added. The reaction mixture was stirred at rt for 20 h under inert atmosphere, filtered over celite, and concentrated in vacuo. The residue was treated with water/DCM. Then, the organic layer was dried over with Na₂SO₄, filtered, and concentrated in vacuum to afford the desired compound.

Procedure M: To a solution of Intermediate (IX) (1 eq), CuSO_{4·}5H₂O (0.4 eq) and sodium ascorbate (0.6 eq) in a mixture water (0.07 M), a solution of alkynyl reagent (2 eq) in THF (0.35 M) was added. The reaction mixture was stirred at rt for 24 h under inert atmosphere, filtered over celite, and concentrated in vacuum. The residue was dissolved in MeOH, treated with IRA-400 resin until pH was basic, filtered, and the solvent was removed under vacuum to afford the desired compounds.

### B.4. Group Transformation Procedure

Procedure N: A mixture of Intermediate (VII) (1 eq) and sodium methoxide 25% (3 eq) in MeOH (0.1 M) was stirred for 18 h at 40° C. The reaction mixture diluted in water/DCM, the organic layer was dried over with Na₂SO₄, filtered, and concentrated in vacuum to afford the desired compound.

### B.5. Azide Reduction Procedure

Procedure Q: To a solution of Intermediate (VIII) (1 eq) in a mixture of THF/water (9:1, 0.08M), DIPEA (3 eq) and PPh₃ (2.6 eq) was added. The reaction mixture was stirred for 4 h at 60° C., then quenched with water, and washed with brine. The organic layer was dried over with Na₂SO₄, filtered, and concentrated in vacuum to afford the desired compound.

### B.6. Amide Formation Procedure

Procedure P: To a solution of Intermediate (XI) or (XII) (1 eq) in DMF (0.1 M), TEA (4 eq) and activated acid (2 eq) were added. The reaction mixture was stirred for 4 h at rt and concentrated in vacuum. The residue was dissolved in MeOH, treated with IRA-400 resin until pH was basic, filtered, and the solvent was removed under vacuum. Finally, the residue was purified by flash chromatography (silica gel) to afford pure compounds.

### B.7. Urea Formation Procedure

Procedure H: Intermediate (XI) or (XII) (1 eq) was dissolved in MeOH and treated with IRA-400 resin until pH was basic, filtered, and the solvent was removed under vacuum. The residue was dissolved in EtOH (0.1 M) and isocyanate reagent (2 eq) was added. The reaction mixture was stirred for 24 h at 60° C. and concentrated in vacuum to afford pure compounds.

### B.8. Carbamate Formation Procedure

Procedure V: Intermediate (XI) or (XII) (1 eq) was dissolved in MeOH and treated with IRA-400 resin until pH was basic. Then, the solution was filtered and the solvent was removed under vacuum. The residue was dissolved in MeOH (1 M) and dimethyl carbonate (40 eq), tetrabutylammonium bromide (0.1 eq) and L-proline (0.1 eq) were added. The reaction mixture was stirred for 48 h at 60° C. and concentrated. The residue was treated with water and DCM and the aqueous layer was concentrated in vacuum. The residue was dissolved in MeOH, treated with DOWEX resin until pH was acid. Then, the solution was filtered and the solvent was removed under vacuum to afford the desired compound.

### B.9. Phosphorylation or Thiophosphorylation Procedures

Procedure G: Intermediate (III), (VI-1) or (IX) (1 eq) was dissolved in DCM (0.02 M) and a solution on tetrazole in ACN (0.43 M) (10-14.4 eq) was added. The mixture was stirred 30 min at rt. Then N,N-diethyl-1,5-dihydrobenzo[e][1,3,2]-dioxaphosphepin-3-amine (5-7.2 eq) was added, and the mixture was stirred at rt overnight. Finally, the reaction mixture was cooled at 0° C. and a solution of tert-butyl hydroperoxide in hexane (5.5 M) (15-19.2 eq) was added. The solution was brought to rt and stirred for 1 h. The mixture was washed with dilute sodium sulfite and extracted with DCM. The organic layer was dried over with Na₂SO₄, filtered, and concentrated in vacuum. The residue was purified by flash chromatography (silica gel) or by crystallization to afford pure compounds.

Procedure T: Intermediate (III) or (IX) (1 eq) was dissolved in DCM (0.02 M) and a solution on tetrazole in ACN (0.43 M) (10-14.4 eq) was added. The mixture was stirred 30 min at rt. Then N,N-diethyl-1,5-dihydrobenzo[e][1,3,2]-dioxaphosphepin-3-amine (5-7.2 eq) was added, and the mixture was stirred at rt overnight. Finally, pyridine (40 eq) and sulfur (40 eq) were added, and the solution was stirred at rt 24 h. The mixture was washed with dilute sodium sulfite and extracted with DCM. The organic layer was dried over with Na₂SO₄, filtered, and concentrated in vacuum. The residue was purified by flash chromatography (silica gel) or by crystallization to afford pure compounds.

### B.10. Phosphate or Thiophosphate Deprotection Procedures

Procedure I: Phosphorylated compound (IV) or (X) was dissolved in a mixture of THF/MeOH/water (3:1:1, 0.01 M) followed by addition of excess palladium hydroxide on carbon. The mixture was placed under hydrogen atmosphere and stirred 2 days at rt. The mixture was then purged with nitrogen, filtered, and concentrated. The compound was brought at pH 9-10 by addition of dilute aqueous NaOH (1 N), and the residue was purified in a sephadex column (PD-10, G-25-M) by eluting with water. All fractions were lyophilized and analyzed by ¹H-RMN. The fractions containing product were purified further in a reverse phase cartridge (Sep-Pack® C18 cartridge, 1 g, Waters Corp., Milford, MA, USA) by eluting with water. All fractions were lyophilized and analyzed by 1H-RMN.

Procedure J: Phosphorylated compound (IV) or (X) was dissolved in a mixture of THF/MeOH/water (3:1:1, 0.01 M) followed by addition of excess palladium hydroxide on carbon. The mixture was placed under hydrogen atmosphere and stirred 2 days at rt. The mixture was then purged with nitrogen, filtered, and concentrated. The compound was brought at pH 10 by addition of dilute aqueous NaOH (1 N), and the solution was stirred for 24-48 h. Finally, the solution was purified on a sephadex column (PD-10, G-25-M) by eluting with water. All fractions were lyophilized and analyzed by ¹H-RMN. The fractions containing product were purified further in a reverse phase cartridge (Sep-Pack® C18 cartridge, 1 g, Waters Corp., Milford, MA, USA) by eluting with water. All fractions were lyophilized and analyzed by ¹H-RMN.

Procedure U: Phosphorylated or thiophosphorylated compound (IV), (IV′) or (X) was treated with thiophenol (40 eq), m-cresol (40 eq) in TFA (0.045 M). Then TBMSBr (40 eq) was added slowly and the mixture was stirred at rt for 4h, quenched with water, and extracted with DCM (3×). The aqueous layer was concentrated in vacuum. The residue was brought at pH 9-10 by addition of water and dilute aqueous NaOH (1N), and the compound was purified on a sephadex column (PD-10, G-25-M) by eluting with water. All fractions were lyophilized and analyzed by ¹H-RMN. The fractions containing product were purified further in a reverse phase cartridge (Sep-Pack® C18 cartridge, 1 g, Waters Corp., Milford, MA, USA) by eluting with water. All fractions were lyophilized and analyzed by ¹H-RMN.

### C. Synthesis of Intermediates II-XIII

### C.1. Intermediates II

4,6-O-bis(5-(Benzyloxy) pentyl)-2-O-tert-butyldimethylsilyl-1,3,5-O-methylidyne-myo-inositol (II-1): According to general alkylation Procedure A, from 1.04 mL (3 eq) of (((5-bromopentyl)oxy)methyl)benzene, 202 mg of (II-1) were obtained (18.3%). HPLC-MS (Condition A): rt=6.30 min; m/z: 657 [M+1]⁺, 679 [M+23]⁺.

4,6-O-bis(10-(Benzyloxy) decyl)-2-O-tert-butyldimethylsilyl-1,3,5-O-methylidyne-myo-inositol (II-2): According to general alkylation Procedure A, from 408 mg (2 eq, in 2 mL of DMF) of (4), 75 mg of (II-2) were obtained (16%). ¹H NMR (400 MHz, Chloroform-d) § 7.39-7.25 (m, 10H), 5.53 (d, J=1.2 Hz, 1H), 4.50 (s, 4H), 4.36 (dt, J=3.6, 1.6 Hz, 1H), 4.25 (q, J=1.6 Hz, 1H), 4.16 (t, J=3.6 Hz, 2H), 4.10 (dt, J=3.6, 1.6 Hz, 2H), 3.53 (dt, J=8.8, 6.4 Hz, 2H), 3.46 (t, J=6.8 Hz, 4H), 3.50 (dt, J=8.8, 6.4 Hz, 2H), 1.61 (p, J=6.8 Hz, 4H), 1.54-1.48 (m, 4H), 1.44-1.16 (m, 24H), 0.94 (s, 9H), 0.14 (s, 6H).

4,6-O-bis(14-(Benzyloxy)tetradecyl)-2-O-tert-butyldimethylsilyl-1,3,5-O-methylidyne-myo-inositol (II-3): According to general alkylation Procedure A, from 312 mg (2 eq, in 1.4 mL of DMF) of (5), 37 mg of (II-3) were obtained (10%). ¹H NMR (400 MHz, Chloroform-d) & 7.34-7.21 (m, 10H), 5.53 (d, J=1.6 Hz, 1H), 4.50 (s, 4H), 4.36 (h, J=1.6 Hz, 1H), 4.25 (q, J=1.6 Hz, 1H), 4.16 (t, J=4 Hz, 2H), 4.11 (dd, J=4, 1.6 Hz, 2H), 3.54 (dt, 8.8, 6.4 Hz, 2H), 3.56 (t, J=6.8 Hz, 4H), 3.45 (dt, J=8.8, 6.4 Hz, 2H), 1.61 (q, J=6.8 Hz, 4H) 1.56-1.59 (m, 4H), 1.30-1.25 (m, 40H), 0.94 (s, 9H), 0.14 (s, 6H).

2-O-tert-Butyldimethylsilyl-4,6-O-bis(3-methoxypropyl)-1,3,5-O-methylidyne-myo-inositol (II-4): According to general alkylation Procedure A, from 0.55 mL (5 eq) of 1-bromo-3-methoxypropane, 232 mg of (II-4) were obtained (52%). HPLC-MS (Condition A): rt=4.58 min; m/z: 449 [M+1]⁺, 471 [M+23]⁺.

2-O-tert-Butyldimethylsilyl-4,6-O-bis(5-methoxypentyl)-1,3,5-O-methylidyne-myo-inositol (II-5): According to general alkylation Procedure A, from 241 mg (2.2 eq, in 3 mL of DMF) of 1-bromo-5-methoxypentane, 21 mg of (II-5) were obtained (7%). HPLC-MS (Condition B): rt=3.80 min; m/z: 505 [M+1]⁺, 527 [M+23]⁺.

2-O-tert-Butyldimethylsilyl-4,6-O-bis(9-methoxynonyl)-1,3,5-O-methylidyne-myo-inositol (II-6): According to general alkylation Procedure A, from 803 mg (2.5 eq, in 1.2 mL of DMF) of (7), 194 mg of (II-6) were obtained (32%). ¹H NMR (400 MHz, Chloroform-d) δ 5.53 (d, J=1.2 Hz, 1H), 4.36 (tt, J=3.2, 1.6 Hz, 1H), 4.24 (dd, J=3.2, 2.4 Hz, 1H), 4.16 (t, J=3.6 Hz, 2H), 4.10 (dt, J=4, 1.6 Hz, 2H), 3.53 (dt, J=8.8, 6.4 Hz, 2H), 3.44 (dt, J=8.8, 6.4 Hz, 2H), 3.36 (t, J=6.4 Hz, 4H), 3.33 (s, 6H), 1.59-1.48 (m, 8H), 1.35-1.26 (m, 20H), 0.94 (s, 9H), 0.14 (s, 6H).

2-O-tert-Butyldimethylsilyl-4,6-O-bis(7-methoxyheptyl)-1,3,5-O-methylidyne-myo-inositol (II-7): According to general alkylation Procedure A, from 780 mg (2.5 eq, in 1.5 mL of DMF) of (6), 398 mg of (II-7) were obtained (47.6%). HPLC-MS (Condition A): rt=6.11 min; m/z: 561 [M+1]⁺, 583 [M+23]⁺.

2-O-tert-Butyldimethylsilyl-1,3,5-O-methylidyne-4,6-O-dipropyl-myo-inositol (II-8): According to general alkylation Procedure A, from 481 μL (5 eq) of 1-iodopropane, 79.14 mg of (II-8) were obtained (21%). ¹H NMR (400 MHz, Chloroform-d) δ 5.52 (d, J=1.4 Hz, 1H), 4.37 (h, J=1.6 Hz, 1H), 4.26 (q, J=1.6 Hz, 1H), 4.16 (t, J=4 Hz, 2H), 4.11 (dt, J=4, 1.6 Hz, 2H), 3.51 (dt, J=8.8, 6.8 Hz, 2H), 3.41 (dt, J=8.8, 6.8 Hz, 3H), 1.55 (h, J=6.8 Hz, 4H), 0.93 (s, 9H), 0.90 (t, J=6.8 Hz, 6H), 0.13 (s, 6H).

2-O-tert-Butyldimethylsilyl-1,3,5-O-methylidyne-4,6-O-dipentyl-myo-inositol (II-9): According to general alkylation Procedure A, from 377 μL (3 eq) of 1-bromopentane, 255 mg of (II-9) were obtained (58%). HPLC-MS (Condition A): rt=6.38 min; m/z: 445 [M+1]⁺.

2-O-tert-Butyldimethylsilyl-4,6-O-diheptyl-1,3,5-O-methylidyne-myo-inositol (II-10): According to general alkylation Procedure A, from 560 μL (5 eq) of 1-bromoheptane, 250 mg of (II-10) were obtained (70%). ¹H NMR (400 MHz, Chloroform-d) δ 5.53 (d, J=1.2 Hz, 1H), 4.36 (h, J=1.6 Hz, 1H), 4.25 (d, J=1.6 Hz, 1H), 4.16 (t, J=4 Hz, 2H), 4.11 (dt, J=4, 1.6 Hz, 2H), 3.54 (dt, J=8.8, 6.4 Hz, 2H), 3.44 (dt, J=8.8, 6.4 Hz, 2H), 1.57-1.49 (m, 4H), 1.37-1.17 (m, 16H), 0.94 (s, 9H), 0.88 (t, J=6.8 Hz, 6H), 0.14 (s, 6H).

2-O-tert-Butyldimethylsilyl-4,6-O-didecyl-1,3,5-O-methylidyne-myo-inositol (II-11): According to general alkylation Procedure A, from 511 μL (3 eq) of 1-bromodecane, 272.6 mg of (II-11) were obtained (57%). ¹H NMR (400 MHz, Chloroform-d) δ 5.53 (d, J=1.2 Hz, 1H), 4.37 (h, J=1.6 Hz, 1H), 4.25 (q, J=1.6 Hz, 1H), 4.16 (t, J=4 Hz, 2H), 4.11 (dt, J=4.0, 1.6 Hz, 2H), 3.54 (dt, J=8.8, 6.4 Hz, 2H), 3.44 (dt, J=8.8, 6.4 Hz, 2H), 1.55-1.50 (m, 4H), 1.37-1.22 (m, 28H), 0.94 (s, 9H), 0.88 (t, J=7.2 Hz 6H), 0.14 (s, 6H).

2-O-tert-Butyldimethylsilyl-1,3,5-O-methylidyne-4,6-O-di(tetradecyl)-myo-inositol (II-12): According to general alkylation Procedure A, from 670 μL (3 eq) of 1-bromotetradecane, 350 mg of (II-12) were obtained (61%). ¹H NMR (400 MHz, Chloroform-d) δ 5.53 (d, J=1.6 Hz, 1H), 4.36 (h, J=1.6 Hz, 1H), 4.25 (q, J=1.6 Hz, 1H), 4.16 (t, J=4 Hz, 2H), 4.11 (dt, J=4, 1.6 Hz, 2H), 3.54 (dt, J=8.8, 6.4 Hz, 2H), 3.44 (dt, J=8.8, 6.4 Hz, 2H), 1.60-1.45 (m, 4H), 1.32-126 (m, 44H), 0.94 (s, 9H), 0.88 (t, J=6.8, 6H), 0.14 (s, 6H).

2-O-tert-Butyldimethylsilyl-4,6-O-bis(5-ethoxycarbonylpentyl)-1,3,5-O-methylidyne-myo-inositol (II-13): According to general alkylation Procedure A, from 351 μL of ethyl 6-bromohexanoate (3 eq), 90 mg of (II-13) were obtained (23%). HPLC-MS (Condition A): rt-5.46 min; m/z: 589 [M+1]⁺, 611 [M+23]⁺.

2-O-tert-Butyldimethylsilyl-4,6-O-bis(10-ethoxycarbonyldecyl)-1,3,5-O-methylidyne-myo-inositol (II-14): According to general alkylation Procedure A, from 455 μL of ethyl 11-bromoundecanoate (2.5 eq), 169 mg of (II-14) were obtained (34%). ¹H NMR (400 MHz, Chloroform-d) δ 5.50 (d, J=1.6 Hz, 1H), 4.34 (h, J=1.6 Hz, 1H), 4.23 (q, J=1.6 Hz, 1H), 4.15-4.07 (m, 8H), 3.52 (dt, J=8.8, 6.4 Hz, 2H), 3.42 (dt, J=8.8, 6.4 Hz, 2H), 2.26 (t, J=7.2 Hz, 4H), 1.59 (p, J=7.2 Hz, 4H), 1.50 (p, J=6.4 Hz, 4H), 1.37-1.17 (m, 30H), 0.92 (s, 9H), 0.12 (s, 6H).

2-O-tert-Butyldimethylsilyl-1,3,5-O-methylidyne-4,6-O-bis(5-(1H-pyrazol-1-yl) pentyl)-myo-inositol (II-15): According to general alkylation Procedure A, from 450 mg of (12), 101 mg of (II-15) were obtained (12%). HPLC-MS (Condition A): rt=4.56 min; m/z: 578 M+1]⁺.

2-O-tert-Butyldimethylsilyl-1,3,5-O-methylidyne-4,6-O-bis(5-(1H-1,2,4-triazol-1-yl) pentyl)-myo-inositol (II-16): According to general alkylation Procedure A, from 500 mg of (14), 60 mg of (II-16) were obtained (6%). HPLC-MS (Condition A): rt=3.70 min; m/z: 579 [M+1]⁺.

### 4,6-O-bis(2-(4-Acetylpiperazin-1-yl)ethyl)-2-O-tert-butyldimethylsilyl-1,3,5-O-methylidyne-myo-inositol (II-17)

Step 1: rac-4-O-(2-(4-Acetylpiperazin-1-yl)ethyl)-2-O-tert-butyldimethylsilyl-1,3,5-O-methylidyne-myo-inositol (34): According to general alkylation Procedure A, from 700 mg (2.5 eq in 7.3 mL of DMF) of (20) (2.5 eq, in 7.3 mL of DMF), 460 mg of (34) were obtained (68%). HPLC-MS (Condition A): rt=1.90 min; m/z: 459 [M+1]⁺.

Step 2: 4,6-O-bis(2-(4-Acetylpiperazin-1-yl)ethyl)-2-O-tert-butyldimethylsilyl-1,3,5-O-methylidyne-myo-inositol (II-17): To a solution of (34) (279 mg, 0.61 mmol) in DMF (0.2 M) at 0° C., NaH (55.7 mg, 1.39 mmol) was added. When the addition was complete, the mixture was stirred for 5 min at rt. Finally, a solution of (20) (309 mg, 1.62 mmol) in DMF (3.2 mL) was added. The reaction was warmed at 90° C., stirred overnight, and concentrated in vacuo. The residue was purified by flash chromatography (silica gel, DCM: MeOH 95:5, 1% NH₃) to afford 89 mg of (II-17) (22%). HPLC-MS (Condition A): rt=1.56 min; m/z: 613 [M+1]⁺.

2-O-tert-Butyldimethylsilyl-1,3,5-O-methylidyne-4,6-O-bis(4,4,4-trifluorobutyl)-myo-inositol (II-18): According to general alkylation Procedure B, from 4.2 mL (3 eq) of 4-bromo-1,1,1-trifluorobutane and 934 mg (10 eq) of LiH, 3.61 g of (II-18) were obtained (60% yield). HPLC-MS (Condition A): rt=5.23 min; m/z: 525 [M+1]⁺.

2-O-tert-Butyldimethylsilyl-1,3,5-O-methylidyne-4,6-O-bis(6,6,6-trifluorohexyl)-myo-inositol (II-19): According to general alkylation Procedure B, from 1.16 g (2.5 eq) of 6-bromo-1,1,1-trifluorohexane and 43 mg (2.5 eq) of LiH, 594 mg of (II-19) were obtained (48%). HPLC-MS (Condition A): rt-6.87 min; m/z: 573 [M+1]⁺, 595 [M+23]⁺.

2-O-tert-Butyldimethylsilyl-1,3,5-O-methylidyne-4,6-O-bis(4-methylpentyl)-myo-inositol (II-20): According to general alkylation Procedure B, from 1 mL (2.5 eq) of 1-bromo-4-methylpentane and 58 mg (2.5 eq) of LiH, 273 mg of (II-20) were obtained (20%). HPLC-MS (Condition A): rt=5.77 min; m/z: 581 [M+1]⁺.

2-O-tert-Butyldimethylsilyl-4,6-O-bis(19-methoxynonadec-10-yn-1-yl)-1,3,5-O-methylidyne-myo-inositol (II-21): According to general alkylation Procedure A, from 2.93 g (2.5 eq) of (31), 1.236 g of (II-21) were obtained (55%). ¹H NMR (400 MHz, Chloroform-d) δ 5.53 (d, J=1.3 Hz, 1H), 4.36 (tt, J=3.6, 1.6 Hz, 1H), 4.25 (q, J=1.6 Hz, 1H), 4.16 (t, J=3.6 Hz, 2H), 4.11-4.09 (m, 2H), 3.53 (dt, J=8.8, 6.4 Hz, 2H), 3.44 (dt, J=8.8, 6.4 Hz, 2H), 3.36 (t, J=6.8 Hz, 4H), 3.33 (s, 6H), 2.19-2.07 (m, 8H), 1.55-1.43 (m, 14H), 1.38-1.26 (m, 38H), 0.94 (s, 9H), 0.14 (s, 6H).

2-O-tert-Butyldimethylsilyl-4,6-O-bis(2-cyclopropylethyl)-1,3,5-O-methylidyne-myo-inositol (II-22): According to general alkylation Procedure B, from 6.24 g (2.2 eq) of (21) and 213 mg (2.2 eq) of LiH, 3.1 g of (II-22) were obtained (59.6%). HPLC-MS (Condition A): rt-5.94 min; 441 [M+1]⁺, 563 [M+23]⁺.

2-O-tert-Butyldimethylsilyl-4,6-O-bis(2-cyclopentylethyl)-1,3,5-O-methylidyne-myo-inositol (II-23): According to general alkylation Procedure B, from 1.53 g (2.5 eq) of (22) and 43 mg (2.5 eq) of LiH, 267 mg of (II-23) were obtained (23.5%). HPLC-MS (Condition A): rt=7.22 min; 497 [M+1]⁺, 519 [M+23]⁺.

2-O-tert-Butyldimethylsilyl-4,6-O-bis(3-(4-methoxyphenyl)propyl)-1,3,5-O-methylidyne-myo-inositol (II-24): According to general alkylation Procedure B, from 1.59 g (2.5 eq) of (23) and 41 mg (2.5 eq) of LiH, 60 mg of (II-24) were obtained (5%). HPLC-MS (Condition A): rt=5.80 min; 601.5 [M+1], 623.5 [M+23]⁺.

2-O-tert-Butyldimethylsilyl-1,3,5-O-methylidyne-4,6-O-bis(3-(3-(trifluoromethyl)phenyl)propyl)-myo-inositol (II-25): According to general alkylation Procedure B, from 1.53 g (2.5 eq) of (24) and 35 mg (2.5 eq) of LiH, 35 mg of (II-25) were obtained (3%). HPLC-MS (Condition A): rt-6.19 min; 677.4 [M+1]⁺, 699.4 [M+23]⁺.

2-O-tert-Butyldimethylsilyl-1,3,5-O-methylidyne-4,6-O-bis(3-(p-tolyl)propyl)-myo-inositol (II-26): According to general alkylation Procedure B, from 1.85 g (2.5 eq) of (25) and 50 mg (2.5 eq) of LiH, 261 mg of (II-26) were obtained (19%). HPLC-MS (Condition A): rt=6.45 min; 569.4 [M+1]⁺, 591.4 [M+23]⁺.

2-O-tert-Butyldimethylsilyl-4,6-O-bis(3-methoxycarbonylproyl)-1,3,5-O-methylidyne-myo-inositol (II-27): According to general alkylation Procedure B, from 2.65 mL (6 eq) of methyl 4-bromobutanoate and 288 mg (10 eq) of LiH, 264 mg of (II-27) were obtained (15%). HPLC-MS (Condition A): rt-4.52 min; 505 [M+1]⁺.

### C.2. Intermediates III

4,6-O-bis(5-(Benzyloxy) pentyl)-myo-inositol (III-1): According to general hydrolysis Procedure C, from 202 mg of (II-1), 163 mg of (III-1) were obtained (>99%). HPLC-MS (Condition A): rt=3.59 min; m/z: 533 [M+1]⁺.

4,6-O-bis(5-Methoxypentyl)-myo-inositol (III-2): According to general hydrolysis Procedure C, from 510 mg of (II-5), 348 mg of (III-2) were obtained (>99%). HPLC-MS (Condition A): rt=2.69 min; m/z: 381 [M+1]⁺, 403 [M+23]⁺.

4,6-O-bis(10-(Benzyloxy) decyl)-myo-inositol (III-3): According to general hydrolysis Procedure D, from 75 mg of (II-2), 51 mg of (III-3) were obtained (80%). 1H NMR (400 MHz, Methanol-d₄) δ 7.33-7.24 (m, 10H), 4.46 (s, 4H), 3.89 (t, J=2.4 Hz, 1H), 3.77 (td, J=6.8, 2.4 Hz, 4H), 3.46 (t, J=6.8 Hz, 4H) 3.40 (t, J=8.4 Hz, 2H), 3.38 (dd, J=8.4, 2.4 Hz, 2H), 3.28 (t, J=8.4 Hz, 1H), 1.65-1.52 (m, 8H), 1.26-1.27 (m, 24H).

4,6-O-bis(14-(Benzyloxy)tetradecyl)-myo-inositol (III-4): According to general hydrolysis Procedure D, from 16.5 mg of (II-3), 14 mg of (III-4) were obtained (>99%). ¹H NMR (400 MHz, Methanol-d₄) δ 7.33-7.27 (m, 10H), 4.46 (s, 4H), 3.90 (t, J=2.4 Hz, 1H), 3.77 (td, J=6.6, 2.8 Hz, 4H), 3.48 (t, J=6.6 Hz, 4H), 3.41 (t, J=9.2 Hz, 2H), 3.35 (dd, J=9.2, 2.8 Hz, 2H), 3.28-3.23 (m, 1H), 1.63-1.56 (m, 8H), 1.45-1.17 (m, 40H).

4,6-O-bis(5-Ethoxycarbonylpentyl)-myo-inositol (III-5): According to general hydrolysis Procedure D, from 114 mg of (II-13), 87.7 mg of (III-5) were obtained (>99%). HPLC-MS (Condition A): rt=2.95 min; m/z: 465 [M+1]⁺.

4,6-O-bis(10-Ethoxycarbonyldecyl)-myo-inositol (III-6): According to general hydrolysis Procedure D, from 166 mg of (II-14), 129 mg of (III-6) were obtained (94%). HPLC-MS (Condition A): rt=4.47 min; m/z: 605 [M+1]⁺.

4,6-O-bis(2-Cyclopropylethyl)-myo-inositol (III-7): According to general hydrolysis Procedure S, from 3.10 g (II-22), 2.23 g of (III-7) were obtained (100%). HPLC-MS (Condition A): rt=2.82 min; m/z: 317 [M+1]⁺, 339 [M+23]⁺.

4,6-O-bis(2-Cyclopentylethyl)-myo-inositol (III-8): According to general hydrolysis Procedure S, from 267 mg of (II-23), 201 mg of (III-8) were obtained (>99%). HPLC-MS (Condition A): rt=3.50 min; m/z: 373 [M+1]⁺, 395 [M+23]⁺.

4,6-O-bis(5-(1H-Pyrazol-1-yl) pentyl)-myo-inositol (III-9): According to general hydrolysis Procedure D, from 90 mg of (II-15), 67 mg (III-9) were obtained (95%). HPLC-MS (Condition A): rt=2.79 min; m/z: 453 [M+1]⁺.

4,6-O-bis(5-(1H-1,2,4-Triazol-1-yl) pentyl)-myo-inositol (III-10): According to general hydrolysis Procedure D, from 19 mg (II-16), 15 mg of (III-10) were obtained (>99%). HPLC-MS (Condition A): rt=2.29 min; m/z: 455 [M+1]⁺.

4,6-O-bis(2-(4-Acetylpiperazin-1-yl)ethyl)-myo-inositol (III-11): According to general hydrolysis Procedure C, from 45 mg of (II-17), 36 mg of (III-11) were obtained (>99%). HPLC-MS (Condition B): rt=0.23 min; m/z: 489 [M+1]⁺, 511 [M+23]⁺.

4,6-O-bis(3-Methoxypropyl)-myo-inositol (III-12): According to general hydrolysis Procedure E, from 232 mg of (II-4), 23 mg of (III-12) were obtained (13%). HPLC-MS (Condition A): rt=1.83 min; m/z: 325 [M+1]⁺.

4,6-O-bis(7-Methoxyheptyl)-myo-inositol (III-13): According to general hydrolysis Procedure E, from 389 mg of (II-7), 321 mg of (III-13) were obtained (>99%). HPLC-MS (Condition A): rt=3.14 min; m/z: 437 [M+1]⁺.

4,6-O-bis(9-Methoxynonyl)-myo-inositol (III-14): According to general hydrolysis Procedure E, from 194 mg of (II-6), 156 mg of (III-14) were obtained (>99%). HPLC-MS (Condition A): rt=3.76 min; m/z: 493 [M+1]⁺, 515 [M+23]⁺.

4,6-O-Dipropyl-myo-inositol (III-15): According to general hydrolysis Procedure E, from 79 mg of (II-8), 26.3 mg of (III-15) were obtained (49%). HPLC-MS (Condition A): rt=1.85 min; m/z: 265 [M+1]⁺, 287 [M+23]⁺.

4,6-O-Dipentyl-myo-inositol (III-16): According to general hydrolysis Procedure E, from 255 mg of (II-9), 107 mg of (III-16) were obtained (59%). HPLC-MS (Condition A): rt=3.00 min; m/z: 321 [M+1]⁺.

4,6-O-Diheptyl-myo-inositol (III-17): According to general hydrolysis Procedure E, from 250 mg of (II-10), 149 mg of (III-17) were obtained (77%). HPLC-MS (Condition A): rt=3.89 min; m/z: 377 [M+1]⁺, 399 [M+23]⁺.

4,6-O-Didecyl-myo-inositol (III-18): According to general hydrolysis Procedure E, from 272 mg of (II-11), 185 mg of (III-18) were obtained (84%). HPLC-MS (Condition A): rt=5.36 min; m/z: 461 [M+1]⁺.

4,6-O-Di(tetradecyl)-myo-inositol (III-19): According to general hydrolysis Procedure E, from 360 mg of (II-12), 212 mg of (III-19) were obtained (69%). ¹H NMR (400 MHz, Chloroform-d) δ 4.19-4.12 (m, 1H), 3.83 (dt, J=9.2, 6.8 Hz, 2H), 3.65 (dt, J=9.2, 6.8 Hz, 2H), 3.52-3.44 (m, 4H), 3.43-3.36 (m, 1H), 1.59 (p, J=6.8 Hz, 4H), 1.33-1.25 (m, 44H), 0.88 (t, J=6.8 Hz, 6H).

4,6-O-bis((1-(2-(Benzyloxycarbonyl)ethyl)-1H-1,2,3-triazol-4-yl)methyl)-myo-inositol (IIII-20): According to general click reaction Procedure K, from 615 mg of (15), 983 mg of (III-20) were obtained (98%). HPLC-MS (Condition A): rt=3.18 min; m/z: 667 [M+1]⁺.

4,6-O-bis(3-(4-(2-(Benzyloxycarbonyl)ethyl)-1H-1,2,3-triazol-1-yl)propyl)-myo-inositol (III-21): According to general click reaction Procedure M, from 70 mg of (IX-3) and 76 mg of benzyl pent-4-ynoate, 98 mg of (III-21) were obtained (67%). In this case, flash chromatography was employed. HPLC-MS (Condition B): rt=2.37 min; m/z: 723 [M+1]⁺

4,6-O-bis(3-(4-(Methoxymethyl)-1H-1,2,3-triazol-1-yl)propyl)-myo-inositol (III-22): According to general click reaction Procedure M, from 41 mg of (IX-3) and 19 μL of 3-methoxyprop-1-yne, 35 mg of (III-22) were obtained (61%). HPLC-MS (Condition A): rt=2.32 min; m/z: 487 [M+1]⁺

4,6-O-bis(4-(4-(Methoxymethyl)-1H-1,2,3-triazol-1-yl)butyl)-myo-inositol (III-23): According to general click reaction Procedure M, from 43 mg of (IX-1) and 19 μL of 3-methoxyprop-1-yne, 13 mg of (III-23) were obtained (22%). HPLC-MS (Condition A): rt=2.40 min; m/z: 515 [M+1]⁺

4,6-O-bis(5-(4-(Methoxymethyl)-1H-1,2,3-triazol-1-yl) pentyl)-myo-inositol (III-24): According to general click reaction Procedure M, from 58 mg of (IX-4) and 12 μL of 3-methoxyprop-1-yne, 50 mg of (III-24) were obtained (64%). HPLC-MS (Condition A): rt=2.48 min; m/z: 543 [M+1]⁺

4,6-O-bis(6-(4-(Methoxymethyl)-1H-1,2,3-triazol-1-yl) hexyl)-myo-inositol (III-25): According to general click reaction Procedure M, from 2 g of (IX-2) and 1.16 mL of 3-methoxyprop-1-yne, 321 mg of (III-25) were obtained (12%). HPLC-MS (Condition A): rt=2.65 min; m/z: 571 [M+1]⁺. Other 2 compounds were isolated in this reaction: 278 mg of 4,6-O-(6-(4-(methoxycarbonyl)-1H-1,2,3-triazol-1-yl) hexyl)-myo-inositol (III-42, 10%) and 454 mg of 4-O-(6-(4-(methoxycarbonyl)-1H-1,2,3-triazol-1-yl) hexyl)-6-O-(6-(4-(methoxymethyl)-1H-1,2,3-triazol-1-yl) hexyl)-myo-inositol (III-43, 17%). III-42: HPLC-MS (Condition A): rt=2.78 min; m/z: 599 [M+1]. III-43: HPLC-MS (Condition A): rt=2.70 min; m/z: 585 [M+1].

4,6-O-bis((1-(3-Methoxypropyl)-1H-1,2,3-triazol-4-yl)methyl)-myo-inositol (III-26): According to general group transformation Procedure N, from 110 mg of (VII-1), 38 mg of (III-26) were obtained (54%). HPLC-MS (Condition A): rt=2.42 min; m/z: 487 [M+1]⁺

4,6-O-bis((1-(6-Methoxyhexyl)-1H-1,2,3-triazol-4-yl)methyl)-myo-inositol (III-27): According to general group transformation Procedure N, from 160 mg of (VII-2), 59 mg of (III-27) were obtained (55%). HPLC-MS (Condition A): rt=2.88 min; m/z: 571 [M+1]⁺

4,6-O-bis((3-(4-Ethyloxycarbonylbutanamido)propyl))-myo-inositol (III-28): According to general amide formation Procedure P, from 160 mg of (XII-1) and 196 mg of (32), 57 mg of (III-28) were obtained (34%). HPLC-MS (Condition B): rt=1.45 min; m/z: 551 [M+1]⁺, 573 [M+23]⁺.

4,6-O-bis(3-(4-Methoxyphenyl)propyl)-myo-inositol (III-29): According to general hydrolysis Procedure S, from 60 mg of (II-24), 47 mg of (III-29) were obtained (>99%). HPLC-MS (Condition A): rt-3.31 min; m/z: 477 [M+1]⁺.

4,6-O-bis(3-(3-(Trifluoromethyl)phenyl)propyl)-myo-inositol (III-30): According to general hydrolysis Procedure S, from 262 mg of (II-25), 212 mg of (III-30) were obtained (84%). HPLC-MS (Condition A): rt=3.88 min; m/z: 553 [M+1]⁺, 575 [M+23]⁺.

4,6-O-bis(3-(p-Tolyl)propyl)-myo-inositol (III-31): According to general hydrolysis Procedure S, from 85 mg of (II-26), 57 mg of (III-31) were obtained (85%). HPLC-MS (Condition A): rt=3.88 min; m/z: 445 [M+1]⁺, 467 [M+23]⁺.

4,6-O-bis(4,4,4-Trifluorobutyl)-myo-inositol (III-32): According to general hydrolysis Procedure S, from 3.61 g of (II-18), 2.73 g of (III-32) were obtained (99%). HPLC-MS (Condition A): rt=3.01 min; m/z: 401 [M+1]⁺, 423 [M+23]⁺.

4,6-O-bis(6,6,6-Trifluorohexyl)-myo-inositol (III-33): According to general hydrolysis Procedure S, from 594 mg of (II-19), 457 mg of (III-33) were obtained (98%). HPLC-MS (Condition A): rt-3.40 min; m/z: 457 [M+1]⁺.

4,6-O-bis(4-Methylpentyl)-myo-inositol (III-34): According to general hydrolysis Procedure S, from 273 mg of (II-20), 193 mg of (III-34) were obtained (96%). HPLC-MS (Condition A): rt=3.40 min; m/z: 349 [M+1]⁺.

4,6-O-bis(19-Methoxynonadec-10-yn-1-yl)-myo-inositol (III-35): According to general hydrolysis Procedure S, from 1.2 g of (II-21), 1.03 g of (III-35) were obtained (>99%). HPLC-MS (Condition A): rt=7.18 min; m/z: 765.6 [M+1]⁺, 787.7 [M+23]⁺.

4,6-O-bis(3-(3-Phenylureido)propyl)-myo-inositol (III-36): According to general urea formation Procedure H, from 89 mg of (XII-1) and 39 μL of phenyl isocyanate, 90 mg of (III-36) were obtained (>99%). HPLC-MS (Condition A): rt=2.70 min; m/z: 533 [M+1]⁺.

4,6-O-bis(3-(3-Cyclopentylureido)propyl)-myo-inositol (III-37): According to general urea formation Procedure H, from 89 mg of (XII-1) and 40.2 μL of isocyanatocyclopentane, 80 mg of (III-37) were obtained (91%). HPLC-MS (Condition A): rt=2.79 min; m/z: 517 [M+1]⁺.

4,6-O-bis(3-((Methoxycarbonyl)amino)propyl)-myo-inositol (III-38): According to general carbamate formation Procedure V, from 74 mg of (XII-1), 7 mg of (III-38) were obtained (12%). HPLC-MS (Condition B): rt-0.25 min; m/z: 411 [M+1]⁺, 433 [M+23]⁺.

4,6-O-bis(5-Acetamidopentyl)-myo-inositol (III-39): According to general hydrolysis Procedure S, from 93 mg of (XIII-1), 52 mg of III-39) were obtained (72%). HPLC-MS (Condition B): rt=0.25 min; m/z: 435 [M+1]⁺, 457 [M+23]⁺.

4,6-O-bis(5-Benzamidopentyl)-myo-inositol (III-40): According to general hydrolysis Procedure S, from 43 mg of (XIII-2), 35 mg of (III-40) were obtained (>99%). HPLC-MS (Condition A): rt=2.92 min; m/z: 559 [M+1]⁺.

4,6-O-bis(5-(Thiophene-2-carboxamido) pentyl)-myo-inositol (III-41): According to general amide formation Procedure P, from 121 mg of (XII-2) and 129 mg of (33), 158 mg of (III-41) were obtained (97%). HPLC-MS (Condition B): rt-2.14 min; m/z: 571 [M+1]⁺.

4,6-O-bis(4-Amino-4-oxobutyl)-myo-inositol (III-44): 185 mg of (III-47) were dissolved 1 mL of ammonia (37% Aq) and the solution was stirred 24 h at 60° C. Then the solution was concentrated in vacuum to afford 98 mg of (III-44) (57%). HPLC-MS (Condition A): rt=2.44 min; m/z: 381 [M+1]⁺, 403 [M+23]⁺.

4,6-O-bis(5-(3-Propylureido) pentyl)-myo-inositol (III-45): According to general urea formation Procedure H, from 89 mg of (XII-1) and 50 μL of 1-isocyanatopropane, 120 mg of (III-45) were obtained (91%). HPLC-MS (Condition A): rt=2.72 min; m/z: 521 [M+1]⁺.

4,6-O-bis(5-((Methoxycarbonyl)amino) pentyl)-myo-inositol (III-46): According to general hydrolysis Procedure S, from 54 mg of (XIII-3), 43 mg of (III-46) were obtained (>99%). HPLC-MS (Condition A): rt=2.62 min; m/z: 467 [M+1]⁺, 490 [M+23]⁺.

4,6-O-bis(3-Methoxycarbonylpropyl)-myo-inositol (III-47): According to general hydrolysis Procedure S, from 274 mg of (II-27), 200 mg of (III-47) were obtained (97%). ¹H NMR (400 MHz, Methanol-d₄) δ 3.79 (br, 1H), 3.74-3.60 (m, 4H), 3.31-3.25 (m, 4H), 3.22 (s, 6H), 2.26-2.19 (m, 4H), 1.76 (p, J=6.8 Hz, 4H).

### C.3. Intermediates IV and IV′

4,6-O-bis(5-(Benzyloxy) pentyl)-1,2,3,5-O-tetrakis(3-oxido-1,5-dihydrobenzo[e][1,3,2]dioxaphosphepin-3-yl)-myo-inositol (IV-26): According to general phosphorylation Procedure G, from 164 mg of (III-1), 173 mg of (IV-26) were obtained (44.6%). HPLC-MS (Condition A): rt=4.85 min; m/z: 1261 [M+1]⁺.

4,6-O-bis(10-(Benzyloxy) decyl)-1,2,3,5-O-tetrakis(3-oxido-1,5-dihydrobenzo[e][1,3,2]dioxaphosphepin-3-yl)-myo-inositol (IV-1): According to general phosphorylation Procedure G, from 50 mg of (III-3), 50 mg of (IV-1) were obtained (48%). HPLC-MS (Condition B): rt=4.21 min; m/z: 1401 [M+1]⁺.

4,6-O-bis(14-(Benzyloxy)tetradecyl)-1,2,3,5-O-tetrakis(3-oxido-1,5-dihydrobenzo[e][1,3,2]dioxaphosphepin-3-yl)-myo-inositol (IV-2): According to general phosphorylation Procedure G, from 16 mg (III-4), 8 mg of (IV-2) were obtained (26%).

4,6-O-bis(3-Methoxypropyl)-1,2,3,5-O-tetrakis(3-oxido-1,5-dihydrobenzo[e][1,3,2]dioxaphosphepin-3-yl)-myo-inositol (IV-3): According to general phosphorylation Procedure G, from 37 mg of (III-12), 25 mg of (IV-3) were obtained (21%). HPLC-MS (Condition A): rt=3.78 min; m/z: 1053 [M+1]⁺.

4,6-O-bis(5-Methoxypentyl)-1,2,3,5-O-tetrakis(3-oxido-1,5-dihydrobenzo[e][1,3,2]dioxaphosphepin-3-yl)-myo-inositol (IV-4): According to general phosphorylation Procedure G, from 444 mg of (III-2), 326 mg of (IV-4) were obtained (25%). HPLC-MS (Condition A): rt=4.03 min; m/z: 1109 [M+1]⁺.

4,6-O-bis(7-Methoxyheptyl)-1,2,3,5-O-tetrakis(3-oxido-1,5-dihydrobenzo[e][1,3,2]dioxaphosphepin-3-yl)-myo-inositol (IV-5): According to general phosphorylation Procedure G, from 365 mg of (III-13), 191 mg of (IV-5) were obtained (19.6%). HPLC-MS (Condition A): rt=4.49 min; m/z: 1165 [M+1]⁺.

4,6-O-bis(7-Methoxynonyl)-1,2,3,5-O-tetrakis(3-oxido-1,5-dihydrobenzo[e][1,3,2]dioxaphosphepin-3-yl)-myo-inositol (IV-6): According to general phosphorylation Procedure G, from 219 mg of (III-14), 71 mg of (IV-6) were obtained (13.1%). HPLC-MS (Condition A): rt=4.98 min; m/z: 1221.6 [M+1]⁺.

1,2,3,5-O-tetrakis(3-Oxido-1,5-dihydrobenzo[e][1,3,2]dioxaphosphepin-3-yl)-4,6-O-dipropyl-myo-inositol (IV-7): According to general phosphorylation Procedure G, from 26.3 mg of (III-15), 24 mg of (IV-7) were obtained (24%). HPLC-MS (Condition A): rt=3.99 min; m/z: 993 [M+1]⁺.

1,2,3,5-O-tetrakis(3-Oxido-1,5-dihydrobenzo[e][1,3,2]dioxaphosphepin-3-yl)-4,6-O-dipentyl-myo-inositol (IV-8): According to general phosphorylation Procedure G, from 105 mg of (III-16), 143 mg of (IV-8) were obtained (42%). HPLC-MS (Condition A): rt-4.39 min; m/z: 1049 [M+1]⁺.

4,6-O-Diheptyl-1,2,3,5-O-tetrakis(3-oxido-1,5-dihydrobenzo[e][1,3,2]dioxaphosphepin-3-yl)-myo-inositol (IV-9): According to general phosphorylation Procedure G, from 150 mg of (III-17), 244 mg of (IV-9) were obtained (55%). HPLC-MS (Condition A): rt=5.04 min; m/z: 1105 [M+1]⁺.

4,6-O-Didecyl-1,2,3,5-O-tetrakis(3-oxido-1,5-dihydrobenzo[e][1,3,2]dioxaphosphepin-3-yl)-myo-inositol (IV-10): According to general phosphorylation Procedure G, from 180 mg of (III-18), 64 mg of (IV-10) were obtained (14%). ¹H NMR (400 MHz, Chloroform-d) δ 7.35-7.09 (m, 16H), 5.41-4.97 (m, 17H), 4.43 (t, J=8.8 Hz, 2H), 4.37 (dt, J=11.2, 4.8 Hz, 1H), 3.81 (t, J=8.8 Hz, 2H), 3.64 (t, J=7.2 Hz, 4H), 1.54 (p, J=7.2 Hz, 4H), 1.25-1.11 (m, 28H), 0.79 (t, J=7.2 Hz, 6H).

1,2,3,5-O-tetrakis(3-Oxido-1,5-dihydrobenzo[e][1,3,2]dioxaphosphepin-3-yl)-4,6-O-di(tetradecyl)-myo-inositol (IV-11): According to general phosphorylation Procedure G, from 134 mg of (III-19), 193 mg of (IV-11) were obtained (63%). HPLC-MS (Condition B): rt-5.25 min; m/z: 1301 [M+1]⁺.

4,6-O-bis(5-Ethoxycarbonylpentyl)-1,2,3,5-O-tetrakis(3-oxido-1,5-dihydrobenzo[e][1,3,2]dioxaphosphepin-3-yl)-myo-inositol (IV-12): According to general phosphorylation Procedure G, from 90 mg of (III-5), 35.5 mg of (IV-12) were obtained (15%). HPLC-MS (Condition A): rt=4.31 min; m/z: 1193 [M+1]⁺.

4,6-O-bis(10-Ethoxycarbonyldecyl)-1,2,3,5-O-tetrakis(3-oxido-1,5-dihydrobenzo[e][1,3,2]dioxaphosphepin-3-yl)-myo-inositol (IV-13): According to general phosphorylation Procedure G, from 129 mg of (III-6), 161 mg of (IV-13) were obtained (57%). HPLC-MS (Condition A): rt=5.38 min; m/z: 1333 [M+1]⁺.

1,2,3,5-O-tetrakis(3-Oxido-1,5-dihydrobenzo[e][1,3,2]dioxaphosphepin-3-yl)-4,6-O-bis(5-(1H-pyrazol-1-yl) pentyl)-myo-inositol (IV-14): According to general phosphorylation Procedure G, from 70 mg of (III-9), 26 mg of (IV-14) were obtained (14%). HPLC-MS (Condition A): rt=3.89 min; m/z: 1182 [M+1]⁺.

1,2,3,5-O-tetrakis(3-Oxido-1,5-dihydrobenzo[e][1,3,2]dioxaphosphepin-3-yl)-4,6-O-bis(5-(1H-1,2,4-triazol-1-yl) pentyl)-myo-inositol (IV-15): According to general phosphorylation Procedure G, from 19 mg of (III-10), 8.8 mg of (IV-15) were obtained. HPLC-MS (Condition A): rt=3.46 min; m/z: 1184 [M+1]⁺.

4,6-O-bis((1-(2-(Benzyloxycarbonyl)ethyl)-1H-1,2,3-triazol-4-yl)methyl)-1,2,3,5-O-tetrakis(3-oxido-1,5-dihydrobenzo[e][1,3,2]dioxaphosphepin-3-yl)-myo-inositol (IV-16): According to general phosphorylation Procedure G, from 423 mg of (III-20), 445 mg of (IV-16) were obtained (50%). HPLC-MS (Condition A): rt=4.16 min; m/z: 1395 [M+1]⁺.

4,6-O-bis((1-(3-Methoxypropyl)-1H-1,2,3-triazol-4-yl)methyl)-1,2,3,5-O-tetrakis(3-oxido-1,5-dihydrobenzo[e][1,3,2]dioxaphosphepin-3-yl)-myo-inositol (IV-17): According to general phosphorylation Procedure G, from 62 mg of (III-26), 64 mg of (IV-17) were obtained (41%). HPLC-MS (Condition A): rt=3.64 min; m/z: 1215 [M+1]⁺.

4,6-O-bis((1-(6-Methoxyhexyl)-1H-1,2,3-triazol-4-yl)methyl)-1,2,3,5-O-tetrakis(3-oxido-1,5-dihydrobenzo[e][1,3,2]dioxaphosphepin-3-yl)-myo-inositol (IV-18): According to general phosphorylation Procedure G, from 59 mg of (III-27), 15 mg of (IV-18) were obtained (11%). HPLC-MS (Condition A): rt=3.95 min; m/z: 1299 [M+1]⁺.

4,6-O-bis(3-(4-(2-(Benzyloxycarbonyl)ethyl)-1H-1,2,3-triazol-1-yl)propyl)-1,2,3,5-O-tetrakis(3-oxido-1,5-dihydrobenzo[e][1,3,2]dioxaphosphepin-3-yl)-myo-inositol (IV-19): According to general phosphorylation Procedure G, from 100 mg of (III-21), 86 mg of (IV-19) were obtained (43%). HPLC-MS (Condition B): rt=3.18 min; m/z: 1451 [M+1]⁺.

4,6-O-bis(3-(4-(Methoxymethyl)-1H-1,2,3-triazol-1-yl)propyl)-1,2,3,5-O-tetrakis(3-oxido-1,5-dihydrobenzo[e][1,3,2]dioxaphosphepin-3-yl)-myo-inositol (IV-20): According to general phosphorylation Procedure G, from 40 mg of (III-22), 7.2 mg of (IV-20) were obtained (7%). HPLC-MS (Condition A): rt=3.48 min; m/z: 1215 [M+1]⁺.

4,6-O-bis(4-(4-(Methoxymethyl)-1H-1,2,3-triazol-1-yl)butyl)-1,2,3,5-O-tetrakis(3-oxido-1,5-dihydrobenzo[e][1,3,2]dioxaphosphepin-3-yl)-myo-inositol (IV-21): According to general phosphorylation Procedure G, from 20 mg of (III-23), 11 mg of (IV-21) were obtained (23%). HPLC-MS (Condition A): rt=3.53 min; m/z: 1243 [M+1]⁺.

4,6-O-bis(5-(4-(Methoxymethyl)-1H-1,2,3-triazol-1-yl) pentyl)-1,2,3,5-O-tetrakis(3-oxido-1,5-dihydrobenzo[e][1,3,2]dioxaphosphepin-3-yl)-myo-inositol (IV-22): According to general phosphorylation Procedure G, from 54 mg of (III-24), 9.7 mg of (IV-22) were obtained (8%). HPLC-MS (Condition A): rt=3.59 min; m/z: 1271 [M+1]⁺.

4,6-O-bis(6-(4-(Methoxymethyl)-1H-1,2,3-triazol-1-yl) hexyl)-1,2,3,5-O-tetrakis(3-oxido-1,5-dihydrobenzo[e][1,3,2]dioxaphosphepin-3-yl)-myo-inositol (IV-23): According to general phosphorylation Procedure G, from 85 mg of (III-25), 35.5 mg of (IV-23) were obtained (18%). HPLC-MS (Condition A): rt=3.66 min; m/z: 1299 [M+1]⁺.

4,6-O-bis(2-(4-Acetylpiperazin-1-yl)ethyl)-1,2,3,5-O-tetrakis(3-oxido-1,5-dihydrobenzo[e][1,3,2]dioxaphosphepin-3-yl)-myo-inositol (IV-24): According to general phosphorylation Procedure G, from 81.5 mg of (III-11), 16 mg of (IV-24) were obtained (8%). HPLC-MS (Condition B): rt=1.92 min; m/z: 1217 [M+1]⁺.

4,6-O-bis((3-(4-Ethyloxycarbonylbutanamido)propyl))-1,2,3,5-O-tetrakis(3-oxido-1,5-dihydrobenzo[e][1,3,2]dioxaphosphepin-3-yl)-myo-inositol (IV-25): According to general phosphorylation Procedure G, from 57 mg of (III-28), 28 mg of (IV-25) were obtained (21%). HPLC-MS (Condition A): rt=2.66 min; m/z: 1279 [M+1]⁺.

4,6-O-bis(2-Cyclopropylethyl)-1,2,3,5-O-tetrakis(3-oxido-1,5-dihydrobenzo[e][1,3,2]dioxaphosphepin-3-yl)-myo-inositol (IV-27): According to general phosphorylation Procedure G, from 2.3 g of (III-7), 3 g of (IV-27) were obtained (39%). HPLC-MS (Condition A): rt=4.30 min; m/z: 1045 [M+1]⁺.

4,6-O-bis(2-Cyclopentylethy)-1,2,3,5-O-tetrakis(3-oxido-1,5-dihydrobenzo[e][1,3,2]dioxaphosphepin-3-yl)-myo-inositol (IV-28): According to general phosphorylation Procedure G, from 200 mg of (III-8), 164 mg of (IV-28) were obtained (28%). HPLC-MS (Condition A): rt-4.74 min; m/z: 1101 [M+1]⁺.

4,6-O-bis(3-(4-Methoxyphenyl)propyl)-1,2,3,5-O-tetrakis(3-oxido-1,5-dihydrobenzo[e][1,3,2]dioxaphosphepin-3-yl)-myo-inositol (IV-29): According to general phosphorylation Procedure G, from 60 mg of (III-29), 36 mg of (IV-29) were obtained (24%). HPLC-MS (Condition A): rt=4.20 min; m/z: 1206 [M+1]⁺.

1,2,3,5-O-tetrakis(3-Oxido-1,5-dihydrobenzo[e][1,3,2]dioxaphosphepin-3-yl)-4,6-O-bis(3-(3-(trifluoromethyl)phenyl)propyl)-myo-inositol (IV-30): According to general phosphorylation Procedure G, from 214 mg of (III-30), 64 mg of (IV-30) were obtained (13%). HPLC-MS (Condition A): rt=4.83 min; m/z: 1281 [M+1]⁺.

1,2,3,5-O-tetrakis(3-Oxido-1,5-dihydrobenzo[e][1,3,2]dioxaphosphepin-3-yl)-4,6-O-bis(3-(p-tolyl)propyl)-myo-inositol (IV-31): According to general phosphorylation Procedure G, from 54.7 mg of (III-31), 19.1 mg of (IV-31) were obtained (13%). HPLC-MS (Condition A): rt=4.76 min; m/z: 1274 [M+1]⁺.

1,2,3,5-O-tetrakis(3-Oxido-1,5-dihydrobenzo[e][1,3,2]dioxaphosphepin-3-yl)-4,6-O-bis(4,4,4-trifluorobutyl)-myo-inositol (IV-32): According to general phosphorylation Procedure G, from 1.62 g of (III-32), 2.34 g of (IV-32) were obtained (51%). HPLC-MS (Condition A): rt=4.29 min; m/z: 1129 [M+1]⁺.

1,2,3,5-O-tetrakis(3-Oxido-1,5-dihydrobenzo[e][1,3,2]dioxaphosphepin-3-yl)-4,6-O-bis(6,6,6-trifluorohexyl)-myo-inositol (IV-33): According to general phosphorylation Procedure G, from 457 mg of (III-33), 414 mg of (IV-33) were obtained (35%). HPLC-MS (Condition A): rt=4.53 min; m/z: 1185 [M+1]⁺.

4,6-O-bis(4-Methylpentyl)-1,2,3,5-O-tetrakis(3-oxido-1,5-dihydrobenzo[e][1,3,2]dioxaphosphepin-3-yl)-myo-inositol (IV-34): According to general phosphorylation Procedure G, from 457 mg of (III-34), 168 mg of (IV-34) were obtained (12%). HPLC-MS (Condition A): rt=4.568 min; m/z: 1077 [M+1]⁺.

4,6-O-bis(19-Methoxynonadec-10-yn-1-yl)-1,2,3,5-O-tetrakis(3-oxido-1,5-dihydrobenzo[e][1,3,2]dioxaphosphepin-3-yl)-myo-inositol (IV-35): According to general phosphorylation Procedure G, from 1.03 g of (III-35), 939 mg of (IV-35) were obtained (45%). HPLC-MS (Condition A): rt=7.19 min; m/z: 1494 [M+1]⁺.

1,2,3,5-O-tetrakis(3-Oxido-1,5-dihydrobenzo[e][1,3,2]dioxaphosphepin-3-yl)-4,6-O-bis(3-(3-phenylureido)propyl)-myo-inositol (IV-36): According to general phosphorylation Procedure G, from 96 mg of (III-36), 187 mg of (IV-36) were obtained (82%). HPLC-MS (Condition A): rt=4.02 min; m/z: 1262 [M+1]⁺.

4,6-O-bis(3-(3-Cyclopentylureido)propyl)-1,2,3,5-O-tetrakis(3-oxido-1,5-dihydrobenzo[e][1,3,2]dioxaphosphepin-3-yl)-myo-inositol (IV-37): According to general phosphorylation Procedure G, from 80 mg of (III-37), 181 mg of (IV-37) were obtained (94%). HPLC-MS (Condition A): rt=3.83 min; m/z: 1245 [M+1]⁺.

4,6-O-bis(3-((Methoxycarbonyl)amino)propyl)-1,2,3,5-O-tetrakis(3-oxido-1,5-dihydrobenzo[e][1,3,2]dioxaphosphepin-3-yl)-myo-inositol (IV-38): According to general phosphorylation Procedure G, from 10 mg of (III-38), 3 mg of (IV-38) were obtained (11%). HPLC-MS (Condition B): rt=2.90 min; m/z: 1139 [M+1]⁺.

4,6-O-bis(5-Acetamidopentyl)-1,2,3,5-O-tetrakis(3-oxido-1,5-dihydrobenzo[e][1,3,2]dioxaphosphepin-3-yl)-myo-inositol (IV-39): According to general phosphorylation Procedure G, from 52 mg of (III-39), 110 mg of (IV-39) were obtained (79%). HPLC-MS (Condition B): rt=2.69 min; m/z: 1163 [M+1]⁺.

4,6-O-bis(5-Benzamidopentyl)-1,2,3,5-O-tetrakis(3-oxido-1,5-dihydrobenzo[e][1,3,2]dioxaphosphepin-3-yl)-myo-inositol (IV-40): According to general phosphorylation Procedure G, from 39 mg of (III-40), 38 mg of (IV-40) were obtained (42%). HPLC-MS (Condition A): rt=4.04 min; m/z: 1288 [M+1]⁺.

1,2,3,5-O-tetrakis(3-Oxido-1,5-dihydrobenzo[e][1,3,2]dioxaphosphepin-3-yl)-4,6-O-bis(5-(thiophene-2-carboxamido) pentyl)-myo-inositol (IV-41): According to general phosphorylation Procedure G, from 158 mg of (III-41), 196 mg of (IV-41) were obtained (54%). HPLC-MS (Condition B): rt=3.13 min; m/z: 1299 [M+1]⁺.

4,6-O-(6-(4-(Methoxycarbonyl)-1H-1,2,3-triazol-1-yl) hexyl)-1,2,3,5-O-tetrakis(3-oxido-1,5-dihydrobenzo[e][1,3,2]dioxaphosphepin-3-yl)-myo-inositol (IV-42): According to general phosphorylation Procedure G, from 278 mg of (III-42), 34 mg of (IV-42) were obtained (5.5%). HPLC-MS (Condition A): rt=3.86 min; m/z: 1327 [M+1]⁺.

4-O-(6-(4-(Methoxycarbonyl)-1H-1,2,3-triazol-1-yl) hexyl)-6-O-(6-(4-(methoxymethyl)-1H-1,2,3-triazol-1-yl) hexyl)-1,2,3,5-O-tetrakis(3-oxido-1,5-dihydrobenzo[e][1,3,2]dioxaphosphepin-3-yl)-myo-inositol (IV-43): According to general phosphorylation Procedure G, from 454 mg of (III-43), 101 mg of (IV-43) were obtained (10%). HPLC-MS (Condition A): rt=3.76 min; m/z: 1313 [M+1]⁺.

4,6-O-bis(4-Amino-4-oxobutyl)-1,2,3,5-O-tetrakis(3-oxido-1,5-dihydrobenzo[e][1,3,2]dioxaphosphepin-3-yl)-myo-inositol (IV-44): According to general phosphorylation Procedure G, from 97.5 mg (III-44), 179 mg of (IV-44) were obtained (59%). HPLC-MS (Condition A): rt=3.29 min; m/z: 1079.5 [M+1]⁺.

1,2,3,5-O-tetrakis(3-Oxido-1,5-dihydrobenzo[e][1,3,2]dioxaphosphepin-3-yl)-4,6-O-bis(5-(3-propylureido) pentyl)-myo-inositol (IV-45): According to general phosphorylation Procedure G, from 120 mg of (III-45), 210 mg of (IV-45) were obtained (73%). HPLC-MS (Condition A): rt=3.73 min; m/z: 1250.5 [M+1]⁺.

4,6-O-bis(5-((Methoxycarbonyl)amino) pentyl)-1,2,3,5-O-tetrakis(3-oxido-1,5-dihydrobenzo[e][1,3,2]dioxaphosphepin-3-yl)-myo-inositol (IV-46): According to general phosphorylation Procedure G, from 50 mg of (III-46), 64 mg of (IV-46) were obtained (50%). HPLC-MS (Condition A): rt=3.86 min; m/z: 1195 [M+1]⁺.

4,6-O-bis(3-methoxycarbonylpropyl)-1,2,3,5-O-tetrakis(3-oxido-1,5-dihydrobenzo[e][1,3,2]dioxaphosphepin-3-yl)-myo-inositol (IV-47): According to general phosphorylation Procedure G, from 70 mg of (III-47), 40 mg of (IV-47) were obtained (19%). HPLC-MS (Condition A): rt=3.77 min; m/z: 1109 [M+1]⁺.

4,6-O-Dipropargyl-1,2,3,5-O-tetrakis(3-oxido-1,5-dihydrobenzo[e][1,3,2]dioxaphosphepin-3-yl)-myo-inositol (IV-48): According to general phosphorylation Procedure G, from 3.92 g of (VI-1), 4.69 g of (IV-48) were obtained (32%). HPLC-MS (Condition A): rt=3.69 min; m/z: 985 [M+1]⁺.

4,6-O-Dipentyl-1,2,3,5-O-tetrakis(3-sulfido-1,5-dihydrobenzo[e][1,3,2]dioxaphosphepin-3-yl)-myo-inositol (IV′-1): According to general thiophosphorylation Procedure T, from 64 mg of (III-16), 28 mg of (IV′-1) were obtained (13%). HPLC-MS (Condition A): rt=5.94 min; m/z: 1113 [M+1]⁺.

4,6-O-bis(5-Methoxypentyl)-1,2,3,5-O-tetrakis(3-sulfido-1,5-dihydrobenzo[e][1,3,2]dioxaphosphepin-3-yl)-myo-inositol (IV′-2): According to general thiophosphorylation Procedure T, from 76 mg of (III-2), 70 mg of (IV′-2) were obtained (30%). HPLC-MS (Condition A): rt=5.49 min; m/z: 1173 [M+1]⁺.

### C.4. Intermediates V

2-O-tert-Butyldimethylsilyl-1,3,5-O-methylidyne-4,6-O-dipropargyl-myo-inositol (V-1): According to general alkylation Procedure A, from 1.0 mL of 3-bromoprop-1-yne in toluene (2.1 eq, 80%), 1.2 g of (V-1) were obtained (>99%). HPLC-MS (Condition A): rt=4.36 min; m/z: 381 [M+1]⁺.

### C.5. Intermediates VI

4,6-O-Dipropargyl-myo-inositol (VI-1): According to general hydrolysis Procedure D, from 1.2 g of (V-1), 740 mg of (VI-1) were obtained (92%). ¹H NMR (400 MHz, Methanol-d₄) δ 4.48 (d, J=2.4 Hz, 4H), 3.89 (t, J=2.8 Hz, 1H), 3.56 (t, J=9.6 Hz, 2H), 3.39 (dd, J=9.6, 2.8 Hz, 2H), 3.33 (d, J=9.6 Hz, 1H), 2.80 (t, J=2.4 Hz, 2H).

### C.6. Intermediates VII

4,6-O-bis((1-(3-(Tosyloxy)propyl)-1H-1,2,3-triazol-4-yl)methyl)-myo-inositol (VII-1): According to general click reaction Procedure L, from 100 mg of (8), 110 mg of (VII-1) were obtained (73%). HPLC-MS (Condition A): rt=3.30 min; m/z: 767 [M+1]⁺.

4,6-O-bis((1-(6-(Tosyloxy) hexyl)-1H-1,2,3-triazol-4-yl)methyl)-myo-inositol (VII-2): According to general click reaction Procedure L, from 116 mg of (17), 162 mg of (VII-2) were obtained (98%). HPLC-MS (Condition A): rt=3.71 min.

### C.7. Intermediates VIII

4,6-O-bis(3-Azidopropyl)-2-O-tert-butyldimethylsilyl-1,3,5-O-methylidyne-myo-inositol (VIII-1): According to general alkylation Procedure A, from 1.27 g (2.1 eq in 5.9 mL DMF) of (8), 249 mg of (VIII-1) were obtained (22%). HPLC-MS (Condition A): rt=5.12 min; m/z: 471 [M+1]⁺.

4,6-O-bis(5-Azidopentyl)-2-O-tert-butyldimethylsilyl-1,3,5-O-methylidyne-myo-inositol (VIII-2): According to general alkylation Procedure A, from 2 g (2.1 eq in 8.4 mL DMF) of (10), 900 mg of (VIII-2) were obtained (51%). HPLC-MS (Condition A): rt=5.62 min; m/z: 527 [M+1], 549 [M+23]⁺.

4,6-O-bis(10-Azidodecyl)-2-O-tert-butyldimethylsilyl-1,3,5-O-methylidyne-myo-inositol (VIII-3): According to general alkylation Procedure A, from 234 mg (in 2 mL DMF, 2.05 eq) of (12), 57.5 mg of (VIII-3) were obtained (27%). ¹H NMR (400 MHz, Chloroform-d) δ 5.52 (d, J=1.6 Hz, 1H), 4.36 (h, J=1.6 Hz, 1H), 4.24 (q, J=1.6 Hz, 1H), 4.16 (t, J=4 Hz, 2H), 4.10 (dt, J=4, 1.6 Hz, 2H), 3.53 (dt, J=8.8, 6.4 Hz, 2H), 3.45 (dt, J=8.8, 6.4 Hz, 2H), 3.25 (t, J=7.0 Hz, 4H), 1.59 (p, J=7.0 Hz, 4H), 1.57-1.46 (m, 4H), 1.38-1.26 (m, 24H), 0.94 (s, 9H), 0.14 (s, 6H).

4,6-O-bis(4-Azidobutyl)-2-O-tert-butyldimethylsilyl-1,3,5-O-methylidyne-myo-inositol (VIII-4): According to general alkylation Procedure A, from 245 mg (2 eq in 1.2 mL of DMF) of (19), 17 mg of (VIII-4) were obtained (7%). HPLC-MS (Condition A): rt=5.33 min; m/z: 499 [M+1]⁺, 521 [M+1]⁺.

4,6-O-bis(6-Azidohexyl)-2-O-tert-butyldimethylsilyl-1,3,5-O-methylidyne-myo-inositol (VIII-5): According to general alkylation Procedure A, from 200 mg (2 eq in 1.6 mL of DMF) of (17), 74 mg of (VIII-5) were obtained (20%). HPLC-MS (Condition A): rt=5.97 min; m/z: 555 [M+1]⁺, 577 [M+1]⁺.

### C.8. Intermediates IX

4,6-O-bis(4-Azidobutyl)-myo-inositol (IX-1): According to general hydrolysis Procedure C introducing a slight modification (aqueous work-up), from 16.7 mg of (VIII-4), 8 mg of (IX-1) were obtained (64%). HPLC-MS (Condition A): rt=2.76 min; m/z: 375 [M+1], 397 [M+23]⁺.

4,6-O-bis(6-Azidohexyl)-myo-inositol (IX-2): According to general hydrolysis Procedure C introducing a slight modification (aqueous work-up), from 74.7 mg of (VIII-5), 40 mg of (IX-2) were obtained (69%). HPLC-MS (Condition A): rt=3.29 min; m/z: 431 [M+1]⁺, 453 [M+23]⁺.

4,6-O-bis(3-Azidopropyl)-myo-inositol (IX-3): According to general hydrolysis Procedure E, from 250 mg of (VIII-1), 174 mg of (IX-3) were obtained (94%). HPLC-MS (Condition A): rt=2.45 min; m/z: 347 [M+1], 369 [M+23]⁺.

4,6-O-bis(5-Azidopentyl)-myo-inositol (IX-4): According to general hydrolysis Procedure D, from 8.9 mg of (VIII-2), 6.8 mg of (IX-4) were obtained (>99%). HPLC-MS (Condition A): rt-3.06 min; m/z: 403 [M+1]⁺, 425 [M+23]⁺.

4,6-O-bis(10-Azidodecyl)-myo-inositol (IX-5): According to general hydrolysis Procedure D, from 57.5 mg of (VIII-3), 47 mg of (IX-5) were obtained (>99%). ¹H NMR (400 MHz, Methanol-d₄) δ 3.92 (t, J=2.6 Hz, 1H), 3.79 (m, 4H), 3.48-3.35 (m, 4H), 3.30-3.25 (m, 5H), 1.64 (p, J=6.8 Hz, 4H), 1.61 (p, J=7.2 Hz, 4H), 1.45-1.27 (m, 24H).

### C.9. Intermediates X

4,6-O-bis(3-Azidopropyl)-1,2,3,5-O-tetrakis(3-oxido-1,5-dihydrobenzo[e][1,3,2]dioxaphosphepin-3-yl)-myo-inositol (X-1): According to general phosphorylation Procedure G, from 305 mg of (IX-3), 148 mg of (X-1) were obtained (16%). HPLC-MS (Condition A): rt=4.01 min; m/z: 1075 [M+1]⁺.

4,6-O-bis(5-Azidopentyl)-1,2,3,5-O-tetrakis(3-oxido-1,5-dihydrobenzo[e][1,3,2]dioxaphosphepin-3-yl)-myo-inositol (X-2): According to general phosphorylation Procedure G, from 300 mg of (IX-4), 530 mg of (X-2) were obtained (63%). HPLC-MS (Condition A): rt=4.32 min; m/z: 1131 [M+1]⁺.

4,6-O-bis(10-Azidodecyl)-1,2,3,5-O-tetrakis(3-oxido-1,5-dihydrobenzo[e][1,3,2]dioxaphosphepin-3-yl)-myo-inositol (X-3): According to general phosphorylation Procedure G, from 100 mg of (IX-5), 63 mg of (X-3) were obtained (27%). HPLC-MS (Condition B): rt=3.86 min; m/z: 1271 [M+1]⁺.

### C.10. Intermediates XI

4,6-O-bis(3-Aminopropyl)-2-O-tert-butyldimethylsilyl-1,3,5-O-methylidyne-myo-inositol (XI-1): According to general azide reduction Procedure Q, from 144 mg of (VIII-1), (XI-1) was obtained pure enough to continue without purification. HPLC-MS (Condition B): rt-2.43 min; m/z: 419 [M+1]⁺, 441 [M+23]⁺.

4,6-O-bis(5-Aminopentyl)-2-O-tert-butyldimethylsilyl-1,3,5-O-methylidyne-myo-inositol (XI-2): According to general azide reduction Procedure Q, from 2.5 g of (VIII-2), 1.4 g of (XI-2) was obtained after column purification (62%). HPLC-MS (Condition A): rt=2.73 min; m/z: 475 [M+1]⁺, 493 [M+23]⁺.

### C.11. Intermediates XII

4,6-O-bis(3-Aminopropyl)-myo-inositol·2TFA (XII-1): According to general hydrolysis Procedure C introducing a slight modification (aqueous work-up), from 128 mg of (XI-1), 160 mg of (XII-1) were obtained (>99%). HPLC-MS (Condition B): rt=0.24 min; m/z: 295 [M+1]⁺.

4,6-O-bis(5-Aminopentyl)-myo-inositol·2HCl (XII-2): According to general hydrolysis Procedure S, from 1.13 g of (XI-2), 447 mg of (XII-2) were obtained (54%). HPLC-MS (Condition B): rt=0.25 min; m/z: 351 [M+1]⁺.

### C.12. Intermediates XIII

4,6-O-bis(5-Acetamidopentyl)-2-O-tert-butyldimethylsilyl-1,3,5-O-methylidyne-myo-inositol (XIII-1): According to general amide formation Procedure P, from 115 mg of (XI-2) and 38 μL of acetyl chloride, 93 mg of (XIII-1) were obtained (69%). HPLC-MS (Condition A): rt=3.67 min; m/z: 559 [M+1]⁺.

4,6-O-bis(5-Benzamidopentyl)-2-O-tert-butyldimethylsilyl-1,3,5-O-methylidyne-myo-inositol (XIII-2): According to general amide formation Procedure P, from 125 mg of (XI-2) and 67 μL of benzoyl chloride, 57 mg of (XIII-2) were obtained (32%). HPLC-MS (Condition B): rt=3.56 min; m/z: 683 [M+1]⁺.

2-O-tert-Butyldimethylsilyl-4,6-O-bis(5-((methoxycarbonyl)amino) pentyl)-1,3,5-O-methylidyne-myo-inositol (XIII-3): According to general carbamate formation Procedure V, from 127 mg of (XI-2), 54 mg of (XIII-3) were obtained (38%). HPLC-MS (Condition A): rt=4.31 min; m/z: 591 [M+1]⁺.

### D. Compound Characterization: Analytical and Spectroscopic Tests

### D.1. NMR

NMR spectra were recorded on an Agilent VNMRS-400 (¹H at 400.10 MHz and ³¹P at 162 MHz). In 1H-NMR chemical shifts were expressed in ppm relative to TMS and coupling constant (J) in Hz. In ³¹P NMR no internal standard was used to collect the phosphorous NMR spectra. The usual internal standard is phosphoric acid, but this was not used due to concerns that it would affect the 1H-NMR.

### D.2. HPLC-MS

Condition A: High-performance Liquid Chromatography (HPLC) 2795 Alliance Waters Aquity coupled to Detector DAD Agilent 1100 and Detector MS Waters ESI triple cuadrupolo Quattro micro, 10 μL of sample in MeOH was injected. Mass spectroscopy (MS) analyzed by FIA (flux injected analysis) coupled to LCT Premier Orthogonal Accelerated Time of Flight Mass Spectrometer, acquiring data by electrospray ionization (ESI) in positive mode. Spectra have been scanned between 50 and 1500 Da with values every 0.2 seconds and peaks are given m/z (% of basis peak). Stationary phase: ZORBAX Extend-C18 3.5 μm 2.1×50 mm (T^{a} 35° C.).

TABLE 3
Condition A, mobile phase

| | Mobile Phase A | Mobile Phase B |
|---|---|---|
| 1 | water + 0.05 formic acid | Acetonitrile + 0.05 formic acid |

TABLE 4
Condition A, gradient

| | Time | Flow | | | |
|---|---|---|---|---|---|
| | (min) | (mL/min) | A (%) | B (%) | Curve |
| 1 | 0.0 | 0.50 | 95.0 | 5.0 | 5 |
| 2 | 0.50 | 0.50 | 95.0 | 5.0 | 5 |
| 3 | 5.50 | 0.50 | 0.0 | 100.0 | 5 |
| 4 | 7.50 | 0.50 | 0.0 | 100.0 | 5 |

Condition B: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample MeOH were injected (c=0.5 mg/mL). Data from mass spectra were analyzed by electrospray ionization in positive and negative mode and peaks are given m/z (% of basis peak). Stationary phase: ZORBAX Extend-C18 3.5 μm 2.1×50 mm (T^{a} 35° C.).

TABLE 5
Condition B, mobile phase

| | Mobile Phase A | Mobile Phase B |
|---|---|---|
| 1 | water + 0.05 formic acid | Acetonitrile + 0.05 formic acid |

TABLE 6
Condition B, gradient

| | Time | Flow | | | |
|---|---|---|---|---|---|
| | (min) | (mL/min) | A (%) | B (%) | Curve |
| 1 | 0.0 | 0.50 | 95.0 | 5.0 | 5 |
| 2 | 0.50 | 0.50 | 95.0 | 5.0 | 5 |
| 3 | 5.50 | 0.50 | 0.0 | 100.0 | 5 |
| 4 | 7.50 | 0.50 | 0.0 | 100.0 | 5 |

Condition C: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample MeOH were injected (c=0.5 mg/mL). Data from mass spectra were analyzed by electrospray ionization in positive and negative mode and peaks are given m/z (% of basis peak). Stationary phase: Xbridge BEH Amide 2.5 μm 4.6×150 mm XP.

TABLE 7
Condition C, Mobile phase

| | Mobile Phase A | Mobile Phase B |
|---|---|---|
| 1 | H₂O + 0.1% DEA + 40 mM AcONH₄ | AcN |

TABLE 8
Condition C, gradient

| | Time | Flow | | | |
|---|---|---|---|---|---|
| | (min) | (mL/min) | A (%) | B (%) | Curve |
| 1 | 0.0 | 1.0 | 5.0 | 95.0 | 5 |
| 2 | 10.00 | 1.0 | 60.0 | 40.0 | 5 |
| 3 | 15.00 | 1.0 | 60.0 | 40.0 | 5 |

Condition D: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample MeOH were injected (c=0.5 mg/mL). Data from mass spectra were analyzed by electrospray ionization in positive and negative mode and peaks are given m/z (% of basis peak). Stationary phase: ZORBAX Extend-C18 3.5 μm 2.1×50 mm (T^{a} 35° C.).

TABLE 9
Condition D, mobile phase

| | Mobile Phase A | Mobile Phase B |
|---|---|---|
| 1 | H₂O + 0.1% DEA + 40 mM AcONH₄ | AcN |

TABLE 10
Condition D, gradient

| | Time | Flow | | | |
|---|---|---|---|---|---|
| | (min) | (mL/min) | A (%) | B (%) | Curve |
| 1 | 0.0 | 0.90 | 99.0 | 1.0 | 5 |
| 2 | 5.00 | 0.90 | 99.0 | 1.0 | 5 |
| 3 | 7.00 | 0.90 | 0.0 | 100.0 | 5 |
| 4 | 10.00 | 0.90 | 0.0 | 100.0 | 5 |

Condition E: HPLC-MS were performed with a High-Performance Liquid Chromatography Thermo Ultimate 3000SD (Thermo Scientific Dionex) coupled to a photodiode array detector and a mass spectrometer LTQ XL ESI-ion trap (Thermo Scientific); 5 μL-20 μL of sample MeOH were injected (c=0.5 mg/mL). Data from mass spectra were analyzed by electrospray ionization in positive and negative mode and peaks are given m/z (% of basis peak). Stationary phase: ZORBAX Extend-C18 3.5 μm 2.1×50 mm (T^{a} 30° C.).

TABLE 11
Condition E, mobile phase

| | Mobile Phase A | Mobile Phase B |
|---|---|---|
| 1 | 0.1% NH₄OH (30%) in water | 0.1% NH₄OH (30%) in ACN |

TABLE 12
Condition E, gradient

| | Time | Flow | | | |
|---|---|---|---|---|---|
| | (min) | (mL/min) | A (%) | B (%) | Curve |
| 1 | 0.0 | 0.60 | 95.0 | 5.0 | 5 |
| 2 | 5.00 | 0.60 | 95.0 | 5.0 | 5 |
| 3 | 7.00 | 0.60 | 0.0 | 100.0 | 5 |
| 4 | 10.00 | 0.60 | 0.0 | 100.0 | 5 |

### Example 1

### Synthesis and Characterization of 4,6-O-bis(5-hydroxypentyl)-myo-inositol-1,2,3,5-tetrakis(phosphate) octasodium salt (Compound 6)

According to general phosphate deprotection Procedure I, from 100 mg of (IV-26), 38 mg of Compound 6 were obtained (56.5%). ¹H NMR (400 MHz, D₂O) δ 4.87-4.80 (m, 1H), 4.55-4-15 (m, 3H), 4.10-3.80 (m, 2H), 3.81-3.71 (m, 4H), 3.45 (t, J=6.6 Hz, 4H), 1.66-1.55 (m, 8H), 1.40 (p, J=7.2 Hz, 4H). ³¹P NMR (162 MHz, D₂O) δ 4.85, 4.41, 4.21. HPLC-MS (Condition C): rt=10.06 min; m/z: 673 [M+1]⁺, 746 [M+1+DEA]⁺, 819 [M+1+2DEA]⁺.

### Example 2

### Synthesis and Characterization of 4,6-O-bis(10-hydroxydecyl)-myo-inositol-1,2,3,5-tetrakis(phosphate) octasodium salt (Compound 7)

According to general phosphate deprotection Procedure I, from 50 mg of (IV-1), 3.15 mg of Compound 7 were obtained (10.9%). ¹H NMR (400 MHz, D₂O) δ 5.08-5.01 (m, 1H), 4.10-4.02 (m, 3H), 3.81-3.75 (m, 6H), 3.60 (t, J=6.6 Hz, 4H), 1.66-1.60 (m, 4H), 1.56-1.52 (m, 4H), 1.30 (br, 24H). ³¹P NMR (162 MHz, D₂O) δ 1.57, −0.15, −1.08. HPLC-MS (Condition C): rt=6.89 min; m/z: 813 [M+1]⁺, 886 [M+1+DEA]⁺, 959 [M+1+2DEA]⁺.

### Example 3

### Synthesis and Characterization of 4,6-O-bis(14-hydroxytetradecyl)-myo-inositol-1,2,3,5-tetrakis(phosphate) octasodium salt (Compound 8)

According to general phosphate deprotection Procedure I, from 8.3 mg of (IV-2), 0.6 mg of Compound 8 were obtained (8%). ¹H NMR (400 MHz, D₂O) δ 4.25-3.71 (m, 3H), 3.69-3.50 (m, 6H), 3.44 (t, J=6.7 Hz, 4H), 1.54-1.29 (m, 8H), 1.15 (m, 40H). HPLC-MS (Condition D): rt=6.8 min; m/z: 998 [M+1+DEA]⁺, 1071 [M+1+2DEA]⁺.

### Example 4

### Synthesis and Characterization of 4,6-O-bis(3-methoxypropyl)-myo-inositol-1,2,3,5-tetrakis(phosphate) octasodium salt (Compound 10)

According to general phosphate deprotection Procedure I, from 23 mg of (IV-3), 17.7 mg of Compound 10 were obtained (99%). ¹H NMR (400 MHz, D₂O) δ 4.35 (br, 4H), 4.14 (br, 2H), 3.82-3.73 (m, 4H), 3.59 (t, J=6.7 Hz, 4H), 3.36 (s, 6H), 1.90 (q, J=6.7, Hz, 4H). ³¹P NMR (162 MHz, D₂O) δ 3.90 (br). HPLC-MS (Condition C): rt=9.96 min; m/z: 718 [M+1+DEA]⁺, 791 [M+1+2DEA].

### Example 5

### Synthesis and Characterization of 4,6-O-bis(5-methoxypentyl)-myo-inositol-1,2,3,5-tetrakis(phosphate) octasodium salt (Compound 9)

According to general phosphate deprotection Procedure I, from 326 mg of (IV-4), 193 mg of Compound 9 were obtained (75%). ¹H NMR (400 MHz, D₂O) δ 4.47-4.36 (m, 3H), 4.31-4.21 (br, 1H) 4.20-4.13 (br, 2H), 3.74-3.66 (m, 4H), 3.50 (t, J=6.8 Hz, 4H), 3.35 (s, 6H), 1.66-154 (m, 8H), 1.38 (p, J=7.6 Hz, 4H). ³¹P NMR (162 MHz, D₂O) 7.46 (br), 6.71 (br). HPLC-MS (Condition C): rt=9.59 min; m/z: 774 [M+1+DEA]⁺, 847 [M+1+2DEA]⁺.

### Example 6

### Synthesis and Characterization of 4,6-O-bis(7-methoxyheptyl)-myo-inositol-1,2,3,5-tetrakis(phosphate) octasodium salt (Compound 11)

According to general phosphate deprotection Procedure I, from 169.5 mg of (IV-5), 29.8 mg of Compound 11 were obtained (22%). ¹H NMR (400 MHz, D₂O) δ 4.96 (d, J=10.6 Hz, 1H), 4.07 (t, J=9.2 Hz, 3H), 3.82-3.70 (m, 6H), 3.49 (t, J=6.8 Hz, 4H), 3.34 (s, 6H), 1.63 (q, J=7 Hz, 4H), 1.59 (q, J=6.8 Hz, 4H), 1.18 (d, J=4.5 Hz, 12H). ³¹P NMR (162 MHz, D₂O) 8 1.92, −0.04, −1.03. HPLC-MS (Condition C): rt=8.95 min; m/z: 757 [M+1]⁺, 830 [M+1+DEA]⁺, 903 [M+1+2DEA]⁺.

### Example 7

### Synthesis and Characterization of 4,6-O-bis(9-methoxynonyl)-myo-inositol-1,2,3,5-tetrakis(phosphate) octasodium salt (Compound 12)

According to general phosphate deprotection Procedure I, from 67 mg of (IV-6), 7.65 mg of Compound 12 were obtained (14% yield). ¹H NMR (400 MHz, D₂O) δ 4.95 (d, J=9.8 Hz, 1H), 4.11-4.02 (m, 3H), 3.89-3.71 (m, 6H), 3.49 (t, J=6.8 Hz, 4H), 3.18 (s, 6H), 1.65-1.55 (m, 8H), 1.31 (br, 20H). ³¹P NMR (162 MHz, D₂O) 8 0.84, −0.36, −1.12. HPLC-MS (Condition C): rt=8.52 min; m/z: 813 [M+1]⁺, 886 [M+1+DEA]⁺, 959 [M+1+2DEA]⁺.

### Example 8

### Synthesis and Characterization of 4,6-O-dipropyl-myo-inositol-1,2,3,5-tetrakis(phosphate) octasodium salt (Compound 4)

According to general phosphate deprotection Procedure I, from 24 mg of (IV-7), 3.8 mg of Compound 4 were obtained (20%). ¹H NMR (400 MHz, D₂O) δ 4.96 (d, J=10.6 Hz, 1H), 4.20-3.96 (m, 3H), 3.75 (t, J=7.2 Hz, 4H), 3.75-371 (m, 2H), 1.63 (sextet, J=7.2 Hz, 4H), 0.90 (t, J=7.2 Hz, 6H). ³¹P NMR (162 MHz, D₂O) δ 1.32, −0.26, −1.18. HPLC-MS (Condition C): rt=10.09 min; m/z: 731 [M+1+2DEA]⁺.

### Example 9

### Synthesis and Characterization of 4,6-O-dipentyl-myo-inositol-1,2,3,5-tetrakis(phosphate) octasodium salt (Compound 1)

According to general phosphate deprotection Procedure I, from 143 mg of (IV-8), 92 mg of Compound 1 were obtained (83%). ¹H NMR (400 MHz, D₂O) δ 5.01-4.91 (m, 1H), 4.10-4.00 (m, 3H), 3.84-3.72 (m, 6H), 1.63 (p, J=7.2 Hz, 4H), 1.36-1.27 (m, 8H), 0.88 (p, J=7.2 Hz, 6H). ³¹P NMR (162 MHz, D₂O) δ 1.32, −0.31, −1.13. HPLC-MS (Condition D): rt-6.16 min; m/z: 714 [M+1]⁺, 787 [M+1+DEA]⁺, 860 [M+1+2DEA]⁺.

### Example 10

### Synthesis and Characterization of 4,6-O-diheptyl-myo-inositol-1,2,3,5-tetrakis(phosphate) octasodium salt (Compound 5)

According to general phosphate deprotection Procedure I, from 244 mg of (IV-9), 7.8 mg of Compound 5 were obtained (4%). ¹H NMR (400 MHz, D₂O) δ 4.10-4.03 (s, 3H), 3.81-3.71 (m, 6H), 1.68-1.59 (m, 4H), 1.41-1.18 (m, 16H), 0.87 (t, J=7.2 Hz, 6H). ³¹P NMR (162 MHz, D₂O) 8 1.96, 0.08, −0.89. HPLC-MS (Condition C): rt=8.46 min; m/z: 843 [M+1+DEA]⁺, 916 [M+1+2DEA]⁺.

### Example 11

### Synthesis and Characterization of 4,6-O-didecyl-myo-inositol-1,2,3,5-tetrakis(phosphate) octasodium salt (Compound 2)

According to general phosphate deprotection Procedure I, from 63 mg of (IV-10), 1.6 mg of Compound 2 were obtained (4%). ¹H NMR (400 MHz, D₂O) δ 5.02 (d, J=8 Hz, 1H), 4.15-3.95 (m, 3H), 3.85-3.70 (m, 6H), 1.67-1.60 (m, 4H), 1.35-1.24 (m, 28H), 0.86 (t, J=6.8 Hz, 6H). HPLC-MS (Condition D): rt=6.89 min; m/z: 781 [M+1], 854 [M+1+DEA]⁺, 927 [M+1+2DEA]⁺, 1000 [M+1+2DEA]⁺.

### Example 12

### Synthesis and Characterization of 4,6-O-di(tetradecyl)-myo-inositol-1,2,3,5-tetrakis(phosphate) octasodium salt (Compound 3)

According to general phosphate deprotection Procedure I, from 1.5 g of (IV-11), 656 mg of Compound 3 were obtained (66.5%). ¹H NMR (400 MHz, D₂O) δ 4.96 (d, J=8.8 Hz, 1H), 4.14-4.02 (m, 3H), 3.84-3.67 (m, 6H), 1.70-1.58 (m, 4H), 1.34-1.26 (m, 44H), 0.86 (t, J=6.8 Hz, 6H). ³¹P NMR (162 MHz, D₂O) δ 4.01, 3.39, 3.02. HPLC-MS (Condition D): 966 [M+1]⁺, 1039 [M+1+DEA]⁺, 1111 [M+1+2DEA]⁺.

### Example 13

### Synthesis and Characterization of 4,6-O-bis(3-aminopropyl)-myo-inositol-1,2,3,5-tetrakis(phosphate) octasodium salt (Compound 13)

According to general phosphate deprotection Procedure I, from 45.6 mg of (X-1), 5.6 mg of Compound 13 were obtained (16.7%). HPLC-MS (Condition C): rt=10.73 min; m/z: 615 [M+1], 688 [M+1+DEA]⁺.

### Example 14

### Synthesis and Characterization of 4,6-O-bis(5-aminopentyl)-myo-inositol-1,2,3,5-tetrakis(phosphate) octasodium salt (Compound 14)

According to general phosphate deprotection Procedure I, from 145 mg of (X-2), 11.3 mg of Compound 14 were obtained (10%). ¹H NMR (400 MHz, D₂O) δ 4.95 (s, 1H), 4.07-4.05 (m, 3H), 3.94 (m, 2H), 3.84-3.70 (m, 7H), 3.02 (t, J=6.7 Hz, 2H), 1.79-1.47 (m, 12H). ³¹P NMR (162 MHz, D₂O) δ 1.86, 0.46, 0.15. HPLC-MS (Condition C): rt=10.77 min; m/z: 671 [M+1]⁺, 744 [M+1+DEA]⁺.

### Example 15

### Synthesis and Characterization of 4,6-O-bis(10-aminodecyl)-myo-inositol-1,2,3,5-tetrakis(phosphate) octasodium salt (Compound 15)

According to general phosphate deprotection Procedure I, from 57.5 mg of (X-3), 0.8 mg of Compound 15 were obtained (2%). ¹H NMR (400 MHz, D₂O) δ 4.95 (d, J=10.3 Hz, 1H), 4.06 (t, J=9.2 Hz, 3H), 3.89-3.67 (m, 7H), 3.59 (t, J=6.8 Hz, 1H), 2.99 (t, J=7.4 Hz, 2H), 1.76-1.57 (m, 8H), 1.45-1.19 (m, 24H). ³¹P NMR (162 MHz, D₂O) 8 2.04, −0.05, −1.00. HPLC-MS (Condition D): rt-6.48 min; m/z: 811 [M+1]⁺, 884 [M+1+DEA]⁺, 958 [M+1+2DEA]⁺.

### Example 16

### Synthesis and Characterization of 4,6-O-bis(5-(1H-pyrazol-1-yl) pentyl)-myo-inositol-1,2,3,5-tetrakis(phosphate) octasodium salt (Compound 16)

According to general phosphate deprotection Procedure I, from 36.6 mg of (IV-14), 8.5 mg of Compound 16 were obtained (29%). ¹H NMR (400 MHz, D₂O) δ 7.64 (d, J=2.4 Hz, 2H), 7.52 (br, 2H), 6.31 (t, J=2.4 Hz, 2H), 4.42 (m, 3H), 4.28-4.14 (m, 3H), 4.13 (t, J=7 Hz, 4H), 3.66 (m, 4H), 1.82 (p, J=7.0 Hz, 4H), 1.67-1.49 (m, 4H), 1.42-1.18 (m, 4H). ³¹P NMR (162 MHz, D₂O) δ 4.91, 4.21. HPLC-MS (Condition C): rt=9.20 min; m/z: 773 [M+1]⁺, 846 [M+1+DEA]⁺, 919 [M+1+2DEA]⁺.

### Example 17

### Synthesis and Characterization of 4,6-O-bis(5-(1H-1,2,4-triazol-1-yl) pentyl)-myo-inositol-1,2,3,5-tetrakis(phosphate) octasodium salt (Compound 17)

According to general phosphate deprotection Procedure I, from 8.8 mg of (IV-15), 5.2 mg of Compound 17 were obtained (74%). ¹H NMR (400 MHz, D₂O) δ 8.46-8.45 (m, 2H), 8.03-8.01 (m, 2H), 4.32-4.23 (m, 5H), 4.12-3.88 (m, 3H), 3.85-3.55 (m, 6H), 1.89 (m, 4H), 1.75-1.53 (m, 4H), 1.31 (p, J=7.4 Hz, 4H). HPLC-MS (Condition C): rt=9.86 min; m/z: 776 [M+1]⁺, 849 [M+1+DEA]⁺, 922 [M+1+2DEA]⁺.

### Example 18

### Synthesis and Characterization of 4,6-O-bis((1-(2-carboxyethyl)-1H-1,2,3-triazol-4-yl)methyl)-myo-inositol-1,2,3,5-tetrakis(phosphate) decasodium salt (Compound 31)

According to general phosphate deprotection Procedure I, from 315 mg of (IV-16), 72 mg of Compound 31 were obtained (39%). ¹H NMR (400 MHz, D₂O) δ 8.22 (s, 2H), 5.08 (d, J=11.6 Hz, 2H), 5.10-4.97 (m, 1H), 4.99 (d, J=11.6 Hz, 2H), 4.70 (t, J=7.2 Hz, 4H), 4.26-4.18 (m, 3H), 3.99 (t, J=9.5 Hz, 2H), 2.88 (t, J=7.2 Hz, 4H). ³¹P NMR (162 MHz, D₂O) δ 1.57, 0.29, −1.00. HPLC-MS (Condition C): rt=10.62 min; m/z: 807 [M+1]⁺, 880 [M+1+DEA]⁺, 953 [M+1+2DEA]⁺.

### Example 19

### Synthesis and Characterization of 4,6-O-bis((1-(3-methoxypropyl)-1H-1,2,3-triazol-4-yl)methyl)-myo-inositol-1,2,3,5-tetrakis(phosphate) octasodium salt (Compound 32)

According to general phosphate deprotection Procedure I, from 64 mg of (IV-17), 22 mg of Compound 32 were obtained (43%). ¹H NMR (400 MHz, D₂O) δ 8.40-7.75 (br, 2H), 5.06-4.80 (m, 5H), 4.55-4.44 (m, 4H), 4.40-3.82 (m, 5H), 3.43 (q, J=6.2 Hz, 4H), 3.32 (s, 6H), 2.12-2.10 (m, 4H). HPLC-MS (Condition C): rt-9.56 min; m/z: 880 [M+1+DEA]⁺.

### Example 20

### Synthesis and Characterization of 4,6-O-bis((1-(6-methoxyhexyl)-1H-1,2,3-triazol-4-yl)methyl)-myo-inositol-1,2,3,5-tetrakis(phosphate) octasodium salt (Compound 33)

According to general phosphate deprotection Procedure I, from 15 mg of (IV-18), 1.8 mg of Compound 33 were obtained (15%). ¹H NMR (400 MHz, D₂O) δ 8.08 (s, 2H), 4.87 (d, J=11.6 Hz, 2H), 4.85-4.75 (m, 1H), 4.81 (d, J=11.6 Hz, 2H), 4.27 (t, J=7 Hz, 4H), 4.07-3.90 (m, 3H), 3.83-3.73 (m, 2H), 3.30 (t, J=6.8 Hz, 4H), 3.17 (s, 6H), 1.76 (p, J=7 Hz, 4H), 1.40 (p, J=6.8 Hz, 4H), 1.17 (m, 8H). HPLC-MS (Condition C): rt=8.83 min; m/z: 891 [M+1]⁺, 964 [M+1+DEA]⁺, 1037 [M+1+2DEA]⁺.

### Example 21

### Synthesis and Characterization of 4,6-O-bis(3-(4-(methoxymethyl)-1H-1,2,3-triazol-1-yl)propyl)-myo-inositol-1,2,3,5-tetrakis(phosphate) octasodium salt (Compound 36)

According to general phosphate deprotection Procedure I, from 7.2 mg of (IV-20), 1.7 mg of Compound 36 were obtained (28%). ¹H NMR (400 MHz, D₂O) δ 8.21 (s, 2H), 5.20 (br, 1H), 4.90-4.61 (m, 4H), 4.61 (s, 4H), 4.20-3.92 (m, 5H), 3.83-3.70 (m, 4H), 3.39 (s, 6H), 2.27-2.18 (m, 4H). HPLC-MS (Condition C): rt-9.73 min; m/z: 880 [M+1+DEA]⁺.

### Example 22

### Synthesis and Characterization of 4,6-O-bis(4-(4-(methoxymethyl)-1H-1,2,3-triazol-1-yl)butyl)-myo-inositol-1,2,3,5-tetrakis(phosphate) octasodium salt (Compound

According to general phosphate deprotection Procedure I, from 11 mg of (IV-21), 1.2 mg of Compound 37 were obtained (28%). ¹H NMR (400 MHz, D₂O) δ 8.08 (s, 2H), 5.20 (br, 1H), 4.61 (s, 4H), 4.55-4.38 (m, 4H), 4.10-3.85 (m, 5H), 3.83-3.70 (m, 4H), 3.39 (s, 6H), 2.05-1.91 (m, 4H), 1.49-1.70 (m, 4H). HPLC-MS (Condition C): rt=9.59 min; m/z: 835 [M+1]⁺, 908 [M+1+DEA]⁺.

### Example 23

### Synthesis and Characterization of 4,6-O-bis(5-(4-(methoxymethyl)-1H-1,2,3-triazol-1-yl) pentyl)-myo-inositol-1,2,3,5-tetrakis(phosphate) octasodium salt (Compound 38)

According to general phosphate deprotection Procedure I, from 11 mg of (IV-22), 1.4 mg of Compound 38 were obtained (16%). ¹H NMR (400 MHz, D₂O) δ 8.07 (s, 2H), 5.05 (br, 1H), 4.61 (s, 4H), 4.45 (t, J=7.6 Hz, 4H), 4.03 (m, 3H), 3.80-3.70 (m, 6H), 3.39 (s, 6H), 1.93 (p, J=7.6 Hz, 4H), 1.67 (p, J=7.6 Hz, 4H), 1.34 (p, J=7.6 Hz, 4H). HPLC-MS (Condition C): rt=9.53 min; m/z: 936 [M+1+DEA]⁺.

### Example 24

### Synthesis and Characterization of 4,6-O-bis(6-(4-(methoxymethyl)-1H-1,2,3-triazol-1-yl) hexyl)-myo-inositol-1,2,3,5-tetrakis(phosphate) octasodium salt (Compound 39)

According to general phosphate deprotection Procedure I, from 35.5 mg of (IV-23), 10 mg of Compound 39 were obtained (34%). ¹H NMR (400 MHz, D₂O) δ 8.04 (s, 2H), 5.30-5.10 (br, 1H), 4.60 (s, 4H), 4.43 (t, J=7.1 Hz, 4H), 4.15-3.97 (m, 3H), 3.83-3.68 (m, 6H), 3.39 (s, 6H), 1.96-1.86 (m, 4H), 1.67-1.57 (m, 4H), 1.41-1.24 (m, 8H). HPLC-MS (Condition C): rt=9.24 min; m/z: 964 [M+1+DEA]⁺.

### Example 25

### Synthesis and Characterization of 4,6-O-bis(2-(4-acetylpiperazin-1-yl)ethyl)-myo-inositol-1,2,3,5-tetrakis(phosphate) octasodium salt (Compound 34)

According to general phosphate deprotection Procedure I, from 16 mg of (IV-24), 6.2 mg of Compound 34 were obtained (48%). ¹H NMR (400 MHz, D₂O) δ 5.10-5.03 (m, 1H), 4.35-4.25 (m, 3H), 4.11-3.85 (m, 12H), 3.65-3.55 (m, 6H), 3.33-3.14 (m, 8H), 2.18 (s, 6H). ³¹P NMR (162 MHz, D₂O) 8 1.32, 0.86, 0.70, 0.24. HPLC-MS (Condition C): rt=10.26 min; m/z: 882 [M+1+DEA]⁺.

### Example 26

### Synthesis and Characterization of 4,6-O-bis((3-(4-carboxybutanamido)propyl))-myo-inositol-1,2,3,5-tetrakis(phosphate) decasodium salt (Compound 45)

According to general phosphate deprotection Procedure I, from 28 mg of (IV-25), 4.4 mg of Compound 45 were obtained (19%). ¹H NMR (400 MHz, D₂O) δ 5.29-4.90 (br, 1H), 4.18-3.92 (m, 3H), 3.89-3.77 (m, 4H), 3.73-360 (m, 2H), 3.37-3.22 (m, 4H), 2.29-2-17 (m, 8H), 1.87-1.70 (m, 8H). HPLC-MS (Condition C): rt=10.18 min; m/z: 916 [M+1+DEA]⁺, 989 [M+1+2DEA]⁺.

### Example 27

### Synthesis and Characterization of 4,6-O-bis(5-carboxypentyl)-myo-inositol-1,2,3,5-tetrakis(phosphate) decasodium salt (Compound 18)

According to general phosphate deprotection Procedure J, from 35.5 mg of (IV-12), 6.8 mg of Compound 18 were obtained (23%). ¹H NMR (400 MHz, D₂O) δ 4.31-4.24 (m, 3H), 4.12-4.08 (m, 1H), 4.01 (br, 2H), 3.58 (dt, J=7.6, 7.2 Hz, 2H), 3.51 (dt, J=7.6, 7.2 Hz, 2H), 2.03 (t, J=7.6, 4H), 1.47 (p, J=7.2 Hz, 4H), 1.42 (p, J=7.6 Hz, 4H), 1.30-1.11 (m, 4H). ³¹P NMR (162 MHz, D₂O) δ 4.86, 4.21. HPLC-MS (Condition C): rt=10.37 min; m/z: 802 [M+1]⁺, 875 [M+1+DEA]⁺, 948 [M+1+2DEA]⁺.

### Example 28

### Synthesis and Characterization of 4,6-O-bis(10-carboxydecyl)-myo-inositol-1,2,3,5-tetrakis(phosphate) decasodium salt (Compound 19)

According to general phosphate deprotection Procedure J, from 187 mg of (IV-13), 34.6 mg of Compound 19 were obtained (22.6%). ¹H NMR (400 MHz, D₂O) 8:4.49 (d, J=12.4 Hz, 1H), 4.42 (br, 1H), 4.40 (br, 1H), 4.25 (d, J=12.4 Hz, 1H), 4.19 (s, 2H), 3.77-3.58 (m, 4H), 2.16 (t, J=7.6 Hz, 4H), 1.66-1.49 (m, 8H), 1.36-1.25 (m, 24H). ³¹P NMR (162 MHz, D₂O) 8:4.79, 4.12, 4.00. HPLC-MS (Condition D): rt=6.26 min; m/z: 942 [M+1]⁺, 1015 [M+1+DEA]⁺, 1088 [M+1+2DEA]⁺.

### Example 29

### Synthesis and Characterization of 4,6-O-bis(3-(4-(2-carboxyethyl)-1H-1,2,3-triazol-1-yl)propyl)-myo-inositol-1,2,3,5-tetrakis(phosphate) decasodium salt (Compound 35)

According to general phosphate deprotection Procedure J, from 74 mg of (IV-19), 11.8 mg of Compound 35 were obtained (21%). ¹H NMR (400 MHz, D₂O) δ 7.89 (s, 2H), 5.28 (br, 1H), 4.66-4.49 (m, 4H), 4.24-3.86 (m, 5H), 3.85-3.55 (m, 4H), 2.94 (t, J=7.8 Hz, 4H), 2.54 (t, J=7.8 Hz, 4H), 2.24-2.15 (m, 4H). ³¹P NMR (162 MHz, D₂O) 8 1.94, 0.53. HPLC-MS (Condition C): rt=10.17 min; m/z: 936 [M+1+DEA]⁺, 1009 [M+1+2DEA]⁺.

### Example 30

### Synthesis and Characterization of 4,6-O-bis(2-cyclopropylethyl)-myo-inositol-1,2,3,5-tetrakis(phosphate) octasodium salt (Compound 28)

According to general phosphate deprotection Procedure I, from 1.34 g of (IV-27), 710 mg of Compound 28 were obtained (68%). ¹H NMR (400 MHz, D₂O) δ 4.51-4.20 (br, 2H), 4.10-3.91 (br, 2H), 3.85-3.63 (br, 5H), 1.46-1.41 (m, 4H), 0.72-0.63 (m, 2H), 0.35-0.31 (m, 4H), 0.00 (q, J=4.7 Hz, 4H). ³¹P NMR (162 MHz, D₂O) δ 3.79− (−0.54) (br). HPLC-MS (Condition C): rt=9.51 min; m/z: 637 [M+1]⁺, 710 [M+1+DEA]⁺, 783 [M+1+2DEA]⁺.

### Example 31

### Synthesis and Characterization of 4,6-O-bis(2-cyclopentylethyl)-myo-inositol-1,2,3,5-tetrakis(phosphate) octasodium salt (Compound 29)

According to general phosphate deprotection Procedure I, from 164 mg of (IV-28), 22 mg of Compound 29 were obtained (17%). ¹H NMR (400 MHz, D₂O) δ 4.39-4.19 (br, 3H), 4.11-3.95 (br, 2H), 3.65 (q, J=8 Hz, 2H), 3.59 (q, J=8 Hz, 2H), 1.76-1.59 (m, 6H), 1.56-1.29 (m, 12H), 1.03-0.97 (m, 4H). ³¹P NMR (162 MHz, D₂O) δ 4.99-3.20 (br). HPLC-MS (Condition C): rt=8.76 min; m/z: 693 [M+1]⁺, 766 [M+1+DEA]⁺, 839 [M+1+2DEA]⁺.

### Example 32

### Synthesis and Characterization of 4,6-O-bis(3-(4-methoxyphenyl)propyl)-myo-inositol-1,2,3,5-tetrakis(phosphate) octasodium salt (Compound 43)

According to general phosphate deprotection Procedure I, from 36 mg of (IV-29), 10.8 mg of Compound 43 were obtained (37%). ¹H NMR (400 MHz, D₂O) δ 7.02 (d, J=8.4 Hz, 4H), 6.72 (d, J=8.4 Hz, 4H), 4.41-4.18 (m, 4H), 4.11 (br, 2H), 3.66 (s, 6H), 3.64 (t, J=7.2 Hz, 4H), 2.47 (t, J=8.0 Hz, 4H), 1.78-1.63 (m, 4H). ³¹P NMR (162 MHz, D₂O) 8 4.29-3.86 (br). HPLC-MS (Condition C): rt=8.55 min; m/z: 797 [M+1]⁺, 870 [M+1+DEA]⁺, 943 [M+1+2DEA]⁺.

### Example 33

### Synthesis and Characterization of 4,6-O-bis(3-(3-(trifluoromethyl)phenyl)propyl)-myo-inositol-1,2,3,5-tetrakis(phosphate) octasodium salt (Compound 44)

According to general phosphate deprotection Procedure I, from 64 mg of (IV-30), 11.9 mg of Compound 44 were obtained (23%). ¹H NMR (400 MHz, D₂O) δ 7.42-7.25 (m, 8H), 4.30-4.06 (m, 5H), 3.69 (s, 4H), 2.62 (t, J=8.1 Hz, 4H), 1.87-1.70 (m, 4H). ³¹P NMR (162 MHz, D₂O) 8 5.09-2.92 (br). HPLC-MS (Condition C): rt=7.84 min; m/z: 873 [M+1]⁺, 946 [M+1+DEA]⁺, 1019 [M+1+2DEA]⁺.

### Example 34

### Synthesis and Characterization of 4,6-O-bis(3-(p-tolyl)propyl)-myo-inositol-1,2,3,5-tetrakis(phosphate) octasodium salt (Compound 42)

According to general phosphate deprotection Procedure I, from 19 mg of (IV-31), 1.1 mg of Compound 42 were obtained (7%). ¹H NMR (400 MHz, D₂O) δ 7.13 (d, J=7.4 Hz, 4H), 7.08 (d, J=7.4 Hz, 4H), 4.02-3.90 (br, 3H), 3.81-3.70 (br, 4H), 3.66-3.57 (br, 2H), 2.57-2.53 (m, 4H), 2.18 (s, 6H), 1.84-1.77 (m, 4H). HPLC-MS (Condition C): rt=8.35 min; m/z: 911 [M+1+2DEA]⁺.

### Example 35

### Synthesis and Characterization of 4,6-O-bis(4,4,4-trifluorobutyl)-myo-inositol-1,2,3,5-tetrakis(phosphate) octasodium salt (Compound 22)

According to general phosphate deprotection Procedure I, from 2.32 g of (IV-32), 820 mg of Compound 22 were obtained (44%). ¹H NMR (400 MHz, D₂O) δ 4.84 (d, J=10.6 Hz, 1H), 3.97-3.90 (m, 3H), 3.74 (t, J=6.5 Hz, 4H), 3.60 (t, J=9.6 Hz, 2H), 2.28-2.15 (m, 4H), 1.79-1.72 (m, 4H). ³¹P NMR (162 MHz, D₂O) 8 1.69, 0.17, −0.92. HPLC-MS (Condition C): rt=8.86 min; m/z: 721 [M+1]⁺, 794 [M+1+DEA]⁺, 867 [M+1+2DEA]⁺.

### Example 36

### Synthesis and Characterization of 4,6-O-bis(6,6,6-trifluorohexyl)-myo-inositol-1,2,3,5-tetrakis(phosphate) octasodium salt (Compound 23)

According to general phosphate deprotection Procedure I, from 248 mg of (IV-33), 45 mg of Compound 23 were obtained (22%). ¹H NMR (400 MHz, D₂O) 8 4.40-3.78 (m, 5H), 3.69-3.56 (m, 4H), 2.13-2.01 (m, 4H), 1.57-1.43 (m, 8H), 1.35-1.28 (m, 4H). HPLC-MS (Condition C): rt=8.27 min; m/z: 923 [M+1+2DEA]⁺.

### Example 37

### Synthesis and Characterization of 4,6-O-bis(4-methylpentyl)-myo-inositol-1,2,3,5-tetrakis(phosphate) octasodium salt (Compound 21)

According to general phosphate deprotection Procedure I, from 167 mg of (IV-34), 4.6 mg of Compound 21 were obtained (3.5%). ¹H NMR (400 MHz, D₂O) 8 4.44-4.34 (m, 2H), 4.25-4.19 (m, 2H), 3.91-3.80 (m, 1H), 3.72-3.50 (m, 4H), 1.68-1.39 (m, 6H), 1.19-1.04 (m, 4H), 0.76 (d, J=6.6 Hz, 12H). HPLC-MS (Condition C): rt=8.74 min; m/z: 742 [M+1+DEA]⁺, 815 [M+1+2DEA]⁺.

### Example 38

### Synthesis and Characterization of 4,6-O-bis(19-methoxynonadec-10-yn-1-yl)-myo-inositol-1,2,3,5-tetrakis(phosphate) octasodium salt (Compound 20)

According to general phosphate deprotection Procedure I, from 410 mg of (III-35), 4.3 mg of Compound 20 were obtained (1.3%). ¹H NMR (400 MHz, D₂O) δ 4.50-4.26 (m, 3H), 4.19-4.02 (m, 2H), 3.70-3.47 (m, 4H), 3.38-3.26 (m, 4H), 3.22 (s, 6H), 1.49-1.41 (m, 8H), 1.25-1.16 (m, 60H). ³¹P NMR (162 MHz, D₂O) δ 4.04 (bs), 3.73 (bs).

### Example 39

### Synthesis and Characterization of 4,6-O-bis(3-(3-phenylureido)propyl)-myo-inositol-1,2,3,5-tetrakis(phosphate) octasodium salt (Compound 46)

According to general phosphate deprotection Procedure I, from 187 mg of (IV-36), 6.5 mg of Compound 46 were obtained (4.3%). ¹H NMR (400 MHz, D₂O) δ 7.27-7.20 (m, 8H), 6.98-6.91 (s, 2H), 4.96-4.78 (m, 1H), 4.03-3.83 (m, 3H), 3.82-3.67 (m, 4H), 3.64-3.45 (m, 2H), 3.27-3.07 (m, 4H), 1.72-1.61 (bs, 4H). HPLC-MS (Condition C): rt=8.73 min; m/z: 926 [M+1+DEA]⁺, 999 [M+1+2DEA]⁺.

### Example 40

### Synthesis and Characterization of 4,6-O-bis(3-(3-cyclopentylureido)propyl)-myo-inositol-1,2,3,5-tetrakis(phosphate) octasodium salt (Compound 47)

According to general phosphate deprotection Procedure I, from 181 mg of (IV-37), 26 mg of Compound 47 were obtained (17.6%). ¹H NMR (400 MHz, D₂O) δ 8 4.87-4.73 (bs, 1H), 3.78-3.64 (m, 5H), 3.62-3.52 (m, 2H), 3.14-3.02 (m, 4H), 1.80-1.71 (m, 4H), 1.65-1.59 (m, 4H), 1.58-1.51 (m, 4H), 1.46-1.39 (m, 4H), 1.32-1.24 (m, 4H). HPLC-MS (Condition C): rt=8.98 min; m/z: 837 [M+1]⁺, 910 [M+1+DEA]⁺, 983 [M+1+2DEA]⁺.

### Example 41

### Synthesis and Characterization of 4,6-O-bis(3-((methoxycarbonyl)amino)propyl)-myo-inositol-1,2,3,5-tetrakis(phosphate) octasodium salt (Compound 24)

According to general phosphate deprotection Procedure I, from 5 mg of (IV-38), 1.7 mg of Compound 24 were obtained (42%). ¹H NMR (400 MHz, D₂O) δ 3.94-3.40 (m, 10H), 3.52 (s, 6H), 3.11 (m, 4H), 1.67 (m, 4H). HPLC-MS (Condition C): rt=9.69 min; m/z: 804 [M+1+DEA]⁺, 877 [M+1+2DEA]⁺.

### Example 42

### Synthesis and Characterization of 4,6-O-bis(5-acetamidopentyl)-myo-inositol-1,2,3,5-tetrakis(phosphate) octasodium salt (Compound 27)

According to general phosphate deprotection Procedure I, from 110 mg of (IV-39), 4.7 mg of Compound 27 were obtained (5%). ¹H NMR (400 MHz, D₂O) δ 4.05-3.83 (m, 3H), 3.78-3.43 (m, 7H), 3.05 (t, J=27.7, 6.7 Hz, 4H), 1.86 (s, 6H), 1.58-1.47 (m, 4H), 1.46-1.38 (m, 4H), 1.34-1.18 (m, 4H). HPLC-MS (Condition C): rt=9.71 min; m/z: 828 [M+1+DEA]⁺, 901 [M+1+2DEA]⁺.

### Example 43

### Synthesis and Characterization of 4,6-O-bis(5-benzamidopentyl)-myo-inositol-1,2,3,5-tetrakis(phosphate) octasodium salt (Compound 48)

According to general phosphate deprotection Procedure I, from 43 mg of (IV-40), 15 mg of Compound 48 were obtained (43%). ¹H NMR (400 MHz, D₂O) δ 7.61-7.55 (m, 4H), 7.47-7.29 (m, 6H), 4.40-4.18 (m, 3H), 4.08-3.88 (m, 2H), 3.61-3.55 (m, 5H), 3.17 (t, J=7 Hz, 4H), 1.56-1.41 (m, 8H), 1.33-1.23 (m, 4H). HPLC-MS (Condition C): rt-8.73 min; m/z: 879 [M+1]⁺, 952 [M+1+DEA]⁺, 1025 [M+1+2DEA]⁺.

### Example 44

### Synthesis and Characterization of 4,6-O-bis(5-(thiophene-2-carboxamido) pentyl)-myo-inositol-1,2,3,5-tetrakis(phosphate) octasodium salt (Compound 49)

According to general phosphate deprotection Procedure U, from 196 mg of (IV-41), 19 mg of Compound 49 were obtained (12%). ¹H NMR (400 MHz, D₂O) 8 7.68-7.37 (m, 4H), 7.15-6.84 (m, 2H), 4.48-4.03 (m, 5H), 3.56 (t, J=8.2, 6.7 Hz, 4H), 3.19-3.12 (m, 4H), 1.50-1.37 (m, 8H), 1.25-1.19 (m, 4H). HPLC-MS (Condition C): rt=8.85 min; m/z: 964 [M+1+DEA]⁺, 1037 [M+1+2DEA]⁺.

### Example 45

### Synthesis and Characterization of 4,6-O-(6-(4-carboxy-1H-1,2,3-triazol-1-yl) hexyl)-myo-inositol-1,2,3,5-tetrakis(phosphate) decasodium salt (Compound 40)

According to general phosphate deprotection Procedure J, from 34 mg of (IV-42), 9.3 mg of Compound 40 were obtained (33%). ¹H NMR (400 MHz, D₂O) δ 8.08 (s, 2H), 4.32-4.22 (m, 8H), 4.10-3.96 (m, 2H), 3.65-3.54 (m, 4H), 1.76 (p, J=7.5 Hz, 4H), 1.54-1.39 (m, 4H), 1.29-1.15 (m, 8H). ³¹P NMR (162 MHz, D₂O) δ 4.81-3.21 (bs). HPLC-MS (Condition C): rt=10.27 min; m/z: 964 [M+1+DEA]⁺, 1037 [M+1+2DEA]⁺.

### Example 46

### Synthesis and Characterization of 4-O-(6-(4-carboxy-1H-1,2,3-triazol-1-yl) hexyl)-6-O-(6-(4-(methoxymethyl)-1H-1,2,3-triazol-1-yl) hexyl)-myo-inositol-1,2,3,5-tetrakis(phosphate) nonasodium salt (Compound 41)

According to general phosphate deprotection Procedure J, from 101 mg of (IV-43), 35 mg of Compound 41 were obtained (42%). ¹H NMR (400 MHz, D₂O) δ 8.05 (s, 1H), 7.86 (s, 1H), 4.42 (s, 2H), 4.37-4.16 (m, 7H), 4.13-4.08 (m, 1H), 4.05 (s, 2H), 3.62-3.50 (m, 4H), 3.22 (s, 3H), 1.83-1.64 (m, 4H), 1.56-1.33 (m, 4H), 1.29-1.19 (m, 4H), 1.18-1.08 (m, 4H). ³¹P NMR (162 MHz, D₂O) δ 4.83, 4.18, 4.10. HPLC-MS (Condition C): rt=9.65 min; m/z: 891 [M+1]⁺, 964 [M+1+DEA]⁺, 1037 [M+1+2DEA]⁺.

### Example 47

### Synthesis and Characterization of 4,6-O-bis(4-amino-4-oxobutyl)-myo-inositol-1,2,3,5-tetrakis(phosphate) octasodium salt (Compound 26)

According to general phosphate deprotection Procedure I, from 168 mg of (IV-44), mg of Compound 26 were obtained (25%). ¹H NMR (400 MHz, D₂O) δ 4.17-3.39 (m, 10H), 2.48-2.17 (m, 3H), 2.16-2.03 (m, 1H), 1.77 (p, J=6.8 Hz, 4H). HPLC-MS (Condition C): rt=10.47 min; m/z: 744 [M+1+DEA]⁺, 817 [M+1+2DEA]⁺

### Example 48

### Synthesis and Characterization of 4,6-O-bis(5-(3-propylureido) pentyl)-myo-inositol-1,2,3,5-tetrakis(phosphate) octasodium salt (Compound 30)

According to general phosphate deprotection Procedure I, from 210 mg of (IV-45), 100 mg of Compound 30 were obtained (58%). ¹H NMR (400 MHz, D₂O) δ 4.41-3.90 (m, 6H), 3.60 (t, J=6.5 Hz, 4H), 3.04-2.85 (m, 8H), 1.61-1.18 (m, 16H), 0.75 (t, J=7.4 Hz, 6H). HPLC-MS (Condition C): rt=9.20 min; m/z: 841 [M+1]⁺, 914 [M+1+DEA]⁺.

### Example 49

### Synthesis and Characterization of 4,6-O-bis(5-((methoxycarbonyl)amino) pentyl)-myo-inositol-1,2,3,5-tetrakis(phosphate) octasodium salt (Compound 25)

According to general phosphate deprotection Procedure I, from 60 mg of (IV-46), 25 mg of Compound 25 were obtained (61% yield). ¹H NMR (400 MHz, D₂O) δ 4.37-3.82 (m, 6H), 3.64 (m, 4H), 3.52 (s, 6H), 3.00 (t, J=7.1 Hz, 4H), 1.64-1.46 (m, 4H), 1.45-1.33 (m, 4H), 1.33-1.17 (m, 4H). HPLC-MS (Condition C): rt=9.33 min; m/z: 787 [M+1]⁺, 860 [M+1+DEA]⁺, 932 [M+1+2DEA]⁺.

### Example 50

### Synthesis and Characterization of 4,6-O-dipentyl-myo-inositol-1,2,3,5-tetrakis(thiophosphate) octasodium salt (Compound 50)

According to general phosphate deprotection Procedure U, from 97 mg of (IV′-1), 2.2 mg of Compound 50 were obtained (3%). ¹H NMR (400 MHz, D₂O) 4.63-4.55 (m, 2H), 4.52-4.44 (m, 2H), 4.34-4.28 (m, 2H), 3.67 (t, J=7.0 Hz, 4H), 1.59-1.48 (m, 4H), 1.27-1.18 (m, 8H), 0.77 (t, J=6.8 Hz, 6H). HPLC-MS (Condition C): rt=9.36 min; m/z: 850 [M+1+2DEA]⁺, 923 [M+1+3DEA]⁺.

### Example 51

### Synthesis and Characterization of 4,6-O-bis(5-Methoxypentyl)-myo-inositol-1,2,3,5-tetrakis(thiophosphate) octasodium salt (Compound 51)

According to general phosphate deprotection Procedure U, from 54 mg of (IV′-2), 2.2 mg of Compound 51 were obtained (5%). ¹H NMR (400 MHz, D₂O) δ 4.65-4.25 (m, 6H), 3.68 (m, 4H), 3.39 (t, J=6.8 Hz, 4H), 3.23 (s, 6H), 1.60-1.47 (m, 8H), 1.30 (m, 4H). ³¹P NMR (162 MHz, D₂O) δ 45.82, 45.22, 44.23. HPLC-MS (Condition C): rt-9.89 min; m/z: 910 [M+1+2DEA]⁺, 983 [M+1+3DEA]⁺.

### Example 52

### Synthesis and Characterization of 4,6-O-bis(3-carboxypropyl)-myo-inositol-1,2,3,5-tetrakis(phosphate) decasodium salt (Compound 52)

According to general phosphate deprotection Procedure J, from 40 mg of (IV-47), 11 mg of Compound 52 were obtained (34%). ¹H NMR (400 MHz, D₂O) δ 4.31-4.24 (m, 3H), 4.12-4.08 (m, 1H), 4.01 (br, 2H), 3.58 (m, 4H), 2.11 (m, 4H), 1.72 (m, 4H). HPLC-MS (Condition C): rt=10.73 min; m/z: 746 [M+1+1DEA]⁺, 819 [M+1+2DEA]⁺.

### Example 53

### Synthesis and Characterization of 4,6-O-Dipropargyl-myo-inositol-1,2,3,5-tetrakis(phosphate) octasodium salt (Compound 53)

According to general phosphate deprotection Procedure U, from 50 mg of (IV-48), 10 mg of Compound 53 were obtained (35%). ¹H NMR (400 MHz, D₂O) δ 4.89-4.81 (m, 1H), 4.43 (d, J=1.9 Hz, 4H), 4.11-3.87 (m, 3H), 3.79-3.60 (m, 2H), 2.77 (t, J=1.9 Hz, 2H). ³¹P NMR (162 MHz, D₂O) δ 1.22, 0.47, −0.86. HPLC-MS (Condition C): rt=10.70 min; m/z: 724 [M+1+2DEA]⁺, 797 [M+1+3DEA]⁺.

### Example 54

### In Vitro Inhibition of Calcium Phosphate Crystallization

The in vitro efficacy of the IP4-4,6 substituted derivatives (e.g., Compounds 1 to 53) of the invention on the inhibition of calcium phosphate crystallization in human plasma samples was evaluated according to a spectrophotometric assay previously described in the art (Ferrer M, et al., Sci Rep 2017; 7:6858, doi:10.1038/s41598-017-07203-x).

A 96-well plate was employed. Plasma was spiked (1 volume of the IP4-4,6 substituted derivatives of the invention per 19 volumes of plasma) with increasing concentrations of the derivatives in the range of 0-100 μM. Plasma was then centrifuged at 10,000 g at rt and subsequently mixed with a mixture of 5 mM hydrogen phosphate and 41.67 mM calcium to attain final concentrations of 1.5 mM phosphate and 12.5 mM calcium, respectively. All reagent solutions were filtered, and pH adjusted to 7.4.

Crystallization of calcium phosphate was monitored spectrophotometrically for 30 min at rt by measuring the increments in absorbance at 550 nm using the Biotek Powerwave XS Microplate spectrophotometer (BioTek Instruments, Inc., Winooski, VT, US). The plate was incubated at rt in an orbital shaker and absorbance measured every 3 min.

Plasma crystallization was assessed based on slope measurement in the linear range between 6 and 24 min from plots of increase in absorbance versus logarithm of time. The efficacy of different myo-inositol phosphates derivatives in preventing in vitro formation of calcium phosphate crystals was assessed in human plasma samples using the slopes obtained between 6- and 24-min. Inhibition of crystallization was measured by comparing the slopes of the control sample (blank plasma) with those of samples containing the inhibitor as shown below:

$$\% Inhibition of crystallization = \frac{Slope ( blank plasma ) - Slope ( inhibitor )}{Slope ( blank plasma )} \cdot 100$$

### See Table 13.

TABLE 13

| | | | Relative |
|---|---|---|---|
| Compound | Family | Example | Potency |
| 6 | A | 1 | +++ |
| 7 | A | 2 | + |
| 10 | A | 4 | +++ |
| 9 | A | 5 | +++ |
| 11 | A | 6 | + |
| 12 | A | 7 | + |
| 4 | A | 8 | +++ |
| 1 | A | 9 | +++ |
| 5 | A | 10 | +++ |
| 2 | A | 11 | + |
| 3 | A | 12 | +++ |
| 13 | A | 13 | ++ |
| 14 | A | 14 | +++ |
| 15 | A | 15 | + |
| 16 | A | 16 | +++ |
| 17 | A | 17 | + |
| 31 | B | 18 | +++ |
| 32 | B | 19 | +++ |
| 33 | B | 20 | ++ |
| 36 | B | 21 | ++ |
| 37 | B | 22 | ++ |
| 38 | B | 23 | +++ |
| 39 | B | 24 | +++ |
| 34 | B | 25 | ++ |
| 45 | C | 26 | +++ |
| 18 | A | 27 | +++ |
| 19 | A | 28 | + |
| 35 | B | 29 | +++ |
| 28 | A | 30 | +++ |
| 29 | A | 31 | +++ |
| 43 | B | 32 | ++ |
| 44 | B | 33 | + |
| 22 | A | 35 | +++ |
| 23 | A | 36 | + |
| 21 | A | 37 | + |
| 20 | A | 38 | +++ |
| 46 | C | 39 | ++ |
| 47 | C | 40 | +++ |
| 24 | A | 41 | + |
| 27 | A | 42 | +++ |
| 48 | C | 43 | + |
| 49 | C | 44 | ++ |
| 40 | B | 45 | +++ |
| 41 | B | 46 | +++ |
| 26 | C | 47 | +++ |
| 30 | A | 48 | +++ |
| 25 | C | 49 | +++ |
| 50 | D | 50 | + |
| 51 | D | 51 | + |
| 52 | A | 52 | +++ |
| 53 | A | 53 | +++ |

Legend:
+++ - EC₅₀ ≤ 5 μM
++ - 5 μM < EC₅₀ ≤ 10 μM
+ - EC₅₀ > 10 μM

### Example 55

### Rat Model Addressing Multiple Ectopic Calcification Related Conditions

The model evaluated the effects of Compound 27 on tissue calcification, blood perfusion, and walking ability in rats for a duration of 13 days. Tissue calcification was induced in groups 2-5 from D1 to D3. Animals induced with calcification were administered placebo or active agent formulations from D1 onwards to assess their effects in preventing calcification. The impact of treatment on tissue calcification, blood perfusion, and walking ability was evaluated. Observations were taken at several points during the treatment from D0 to D13. All animals were weighed every day before treatment.

Fifty-eight male Sprague Dawley (SD) rats (Envigo Corp., Huntingdon, GB) weighing approximately 275-300 g were used. The animals were fed with a standard LASQCdiet® Rod14-H4 diet (LASvendi, Soest, DE). The animals were divided in 5 groups, 10 to 12 animals per group, as follows:
Group 1—Control (sham), 10 animals
Group 2—Physiological saline solution (placebo) daily, 12 animals
Group 3—Compound 27, 5 mg/kg daily, 12 animals
Group 4—Compound 27, 15 mg/kg daily, 12 animals
Group 5—Compound 27, 45 mg/kg daily, 12 animals

Calcification was induced in the animals of groups 2-5 by subcutaneous administration of 120,000 IU/kg vitamin D₃ (cholecalciferol, Duphafral D₃ 1000; Zoetis Inc., Parsippany, NJ, US) in physiological saline 0.9% (w/v) NaCl solution (2 mL/kg) daily from D1 to D3. Animals in group 1 were administered 2 mL/kg of physiological saline solution daily from D1 to D3 via subcutaneous route with no vitamin D3. Animals in group 2 were administered 2 mL/kg of physiological saline solution daily from D1 to D13 via subcutaneous route. Animals in groups 3, 4, and 5 were administered 5 mg/kg, 15 mg/kg, and 45 mg/kg, respectively, of Compound 27 (754.45 g/mol free acid) daily in physiological saline solution (2 mL/kg) from D1 to D13 via subcutaneous route.

### 1. Tissue Analysis

All the living animals of groups 1-5 were sacrificed in D13 for determining their Ca values in tissue (i.e., aorta, carotids, femoral, heart, kidney).

The animals were exsanguinated before sacrifice. Their necropsies were performed, and their whole right and left femoral arteries, left and right carotids, aorta, heart, and right kidney were collected for calcium content measurement. The tissues were lyophilized for 24 h and weighed. The lyophilized tissues were then digested using a 1:1 HNO₃:HClO₄ mixture in a dry bath incubator for 2-4 h at 180° C. The digested tissues were subsequently diluted using ultra-pure Milli-Q water (MilliporeSigma, Merck KGaA, Burlington, MA, US) to a final volume of 5 mL (for femoral and carotid) or 10 mL (for heart, kidney and aorta). Calcium content was quantified via inductively coupled plasma optical emission spectrometry (ICP-OES) using an Optima 7300 DV ICP-OES System spectrometer (PerkinElmer, Inc., Waltham, MA, US) according to the manufacturer's instructions.

### 2. Blood Perfusion

Blood perfusion (BP) was evaluated by laser doppler perfusion imaging applied to the posterior limbs of the animals using a PeriCam PSI Normal Resolution analyzer (Perimed A B, Järfälla, S E). Measurements were taken for all groups at D0, D4, and D13. The perfusion ratio (i.e., mean of the two legs) was calculated by comparing the baseline and either D4 or D13 readings for each group. In addition, the perfusion ratio was calculated by comparing group 1 and groups 2-5.

### 3. Walking Ability

Maximal walking time (MWT) and maximal walking distance (MWD) were assessed by forced incremental treadmill running tests using a two-lane touch screen treadmill device for rats (Cat. No. LE8709TS, Panlab, Harvard Biosciences, Inc., Holliston, MA, US) with a 15% inclination. Measurements were taken for all groups at D0, D6, and D11.

The animals were acclimatized to the treadmill for two days before conducting the test. On the first day, animals were exercised for 5 to 10 minutes with the treadmill speed ranging progressively from 15 m/min to 24 m/min. On the second day, the animals were exercised for 20 minutes as follows: (a) at 15 m/min for the first 5 minutes, (b) at 19.8 m/min for the second 5 minutes and (c) at 24 m/min for the last 10 minutes. Preoperative walking time and distance recordings were obtained. Animals that did not comply with the protocol were excluded from the test. Forty-eight rats (48) were screened for the walking ability test. The selected animals were further exercised for 5-10 minutes the day before the D0 test for ensuring their treadmill habituation. The protocol for MWT and MWD measurements at D0, D5, and D10 was as follows:

| Speed (m/min) | 15 | 19.8 | 24 |
|---|---|---|---|
| Time (min) | 0-5 | 5-10 | 10-40 |

For group 2, the treadmill assessment started 15 min after dosing. For groups 2-5, the treadmill assessment started 25 min after dosing. Rats were kept running for 40 min or until fatigue. The test was stopped when the rats remained on the shock grid for five continuous seconds (i.e., fatigue). MWD (in meters) and MWT (in minutes) were recorded for each animal.

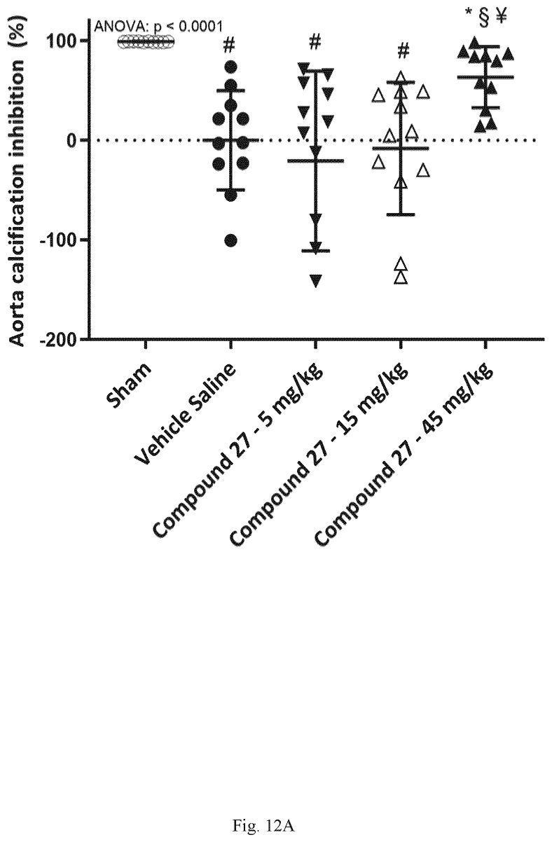

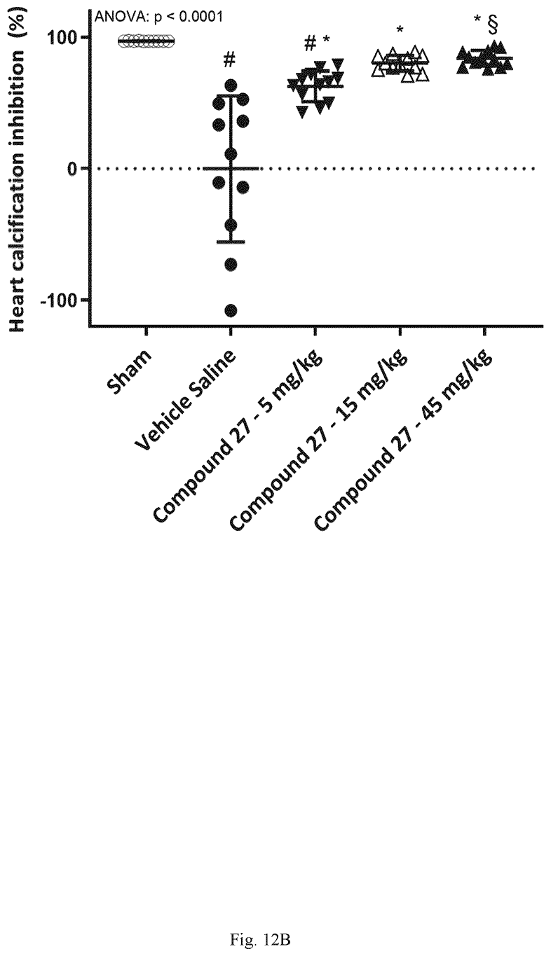

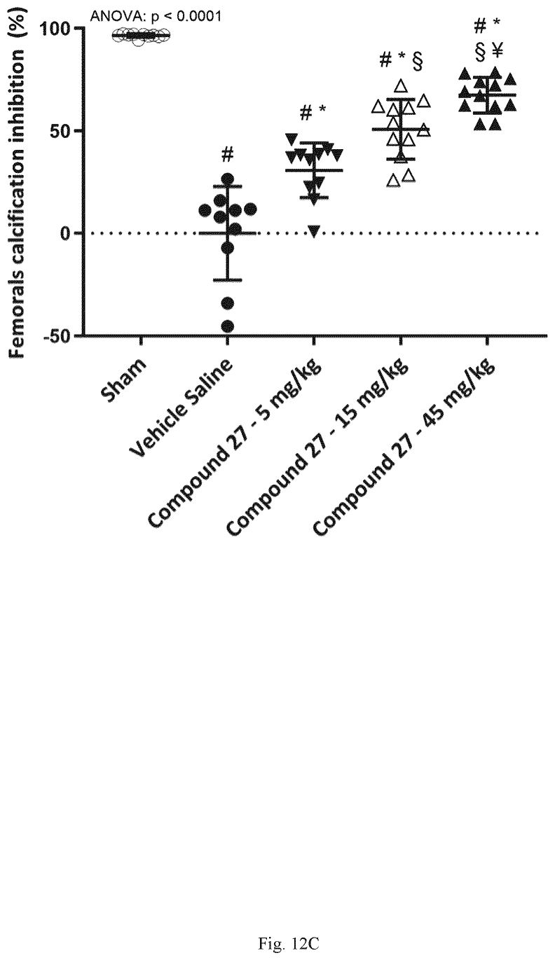

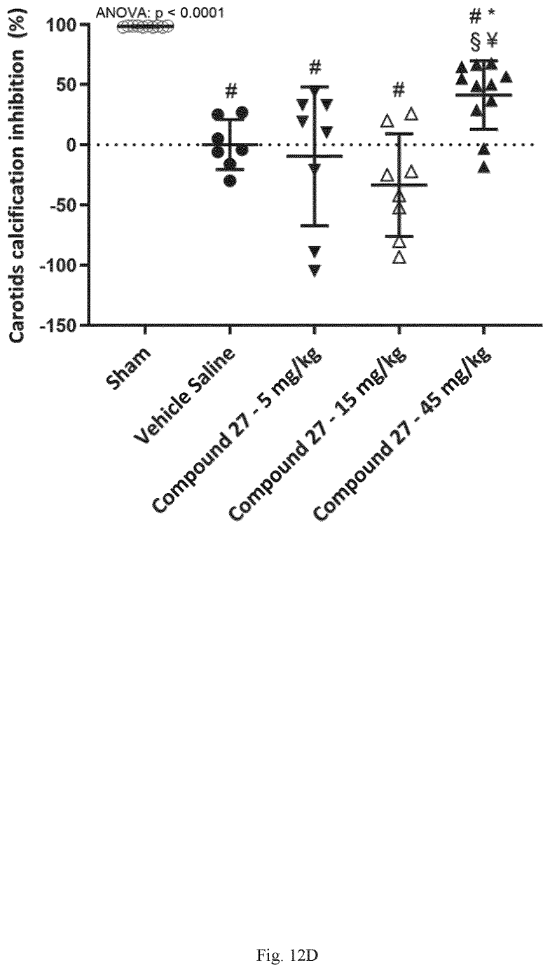

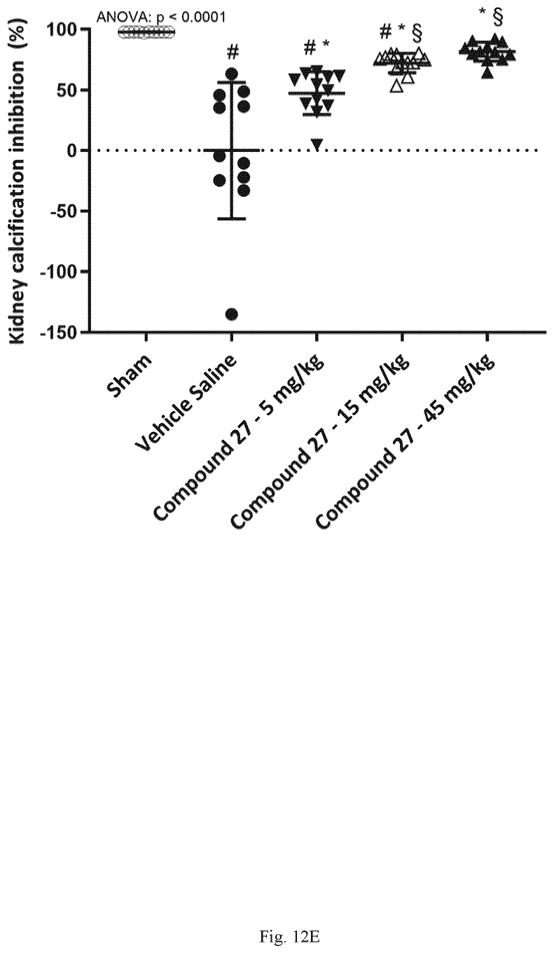

The administration of Compound 27 at doses 15 and 45 mg/kg improves the walking ability of rats administered with vitamin D as early as D5 after the induction of the PAD disease, in a dose-response manner. The results show that Compound 27 inhibits ectopic calcification in all the evaluated tissues. See FIG. 12A to 12E.

The inhibition of calcification in aorta (FIG. 12A) was observed at 45 mg/kg. This result indicated that Compound 27 could be used to treat conditions such as aortic calcification, aortic valve calcification, aortic stenosis, calcific aortic valve stenosis, atherosclerosis, arteriosclerosis, aneurysm or coral reef aorta. Arterial calcification leads to stiffening of the vessels, indicating the potential use of Compound 27 in arterial stiffness. Stiffening of the aorta leads to decreased filling of the coronary vessels during diastole and reduced perfusion of the myocardium, indicating that Compound 27 could be used in coronary disease or coronary artery disease. Decreased coronary filling can lead to the inability of the myocardium to adequately respond to increased oxygen demand resulting in ischemia, even in the absence of coronary artery obstruction. Inadequate response to increased myocardial oxygen demand can lead to symptoms of myocardial ischemia and angina. Hence, Compound 27 could be used in myocardial ischemia and angina pectoris (including chronic stable angina) due to its impact on aortic calcification and consequently, all the downstream effects towards ischemia and chest pain. Arterial stiffening also forces the heart to pump harder and hypertrophy, indicating that Compound 27 could be used in left ventricular hypertrophy. Ultimately, arterial stiffening and heart hypertrophy may lead to arrhythmias, heart failure, congestive heart failure, cardiac disease, cardiovascular mortality or cardiac death, indicating that Compound 27 could be used in these situations or conditions.

The inhibition of calcification in the heart (FIG. 12B) showed a dose-response effect from 5 mg/kg. This result indicated that Compound 27 could be used conditions associated with cardiovascular calcification, coronary calcification, valvular calcification, calcific aortic valve stenosis, atherosclerosis, arteriosclerosis, myocardial infarction, aneurysm, coronary disease or coronary artery disease. Calcification of the coronaries in the heart leads to stiffening of the vessels, indicating the potential use of Compound 27 in arterial stiffness. Stiffening leads to decreased filling of the coronary vessels during diastole and reduced perfusion of the myocardium, indicating that Compound 27 could be used in coronary disease or coronary artery disease. Decreased coronary filling can lead to the inability of the myocardium to adequately respond to increased oxygen demand resulting in ischemia, even in the absence of coronary artery obstruction. Inadequate response to increased myocardial oxygen demand can lead to symptoms of myocardial ischemia and angina. Hence, Compound 27 could be used in myocardial ischemia and angina pectoris (including chronic stable angina) due to its impact on coronary artery calcification in the heart and consequently, all the downstream effects towards ischemia and chest pain. Arterial stiffening also forces the heart to pump harder and hypertrophy, indicating that Compound 27 could be used in left ventricular hypertrophy. Ultimately, coronary artery stiffening, and heart hypertrophy are settings for arrhythmias, heart failure, congestive heart failure, cardiac disease, cardiovascular mortality or cardiac death, indicating that Compound 27 could be used in these situations or conditions.

The inhibition of calcification in the carotids (FIG. 12D) was observed at 45 mg/kg. This result indicated that Compound 27 could be used in situations or conditions of atherosclerosis or arteriosclerosis. Arterial calcification leads to stiffening of the vessels, indicating the potential use of Compound 27 in arterial stiffness.

The inhibition of calcification in the kidneys (FIG. 12E) showed a dose-response effect from 5 mg/kg. This result indicated that Compound 27 could be used in situations or conditions with nephrocalcinosis or renal lithiasis (kidney stones).

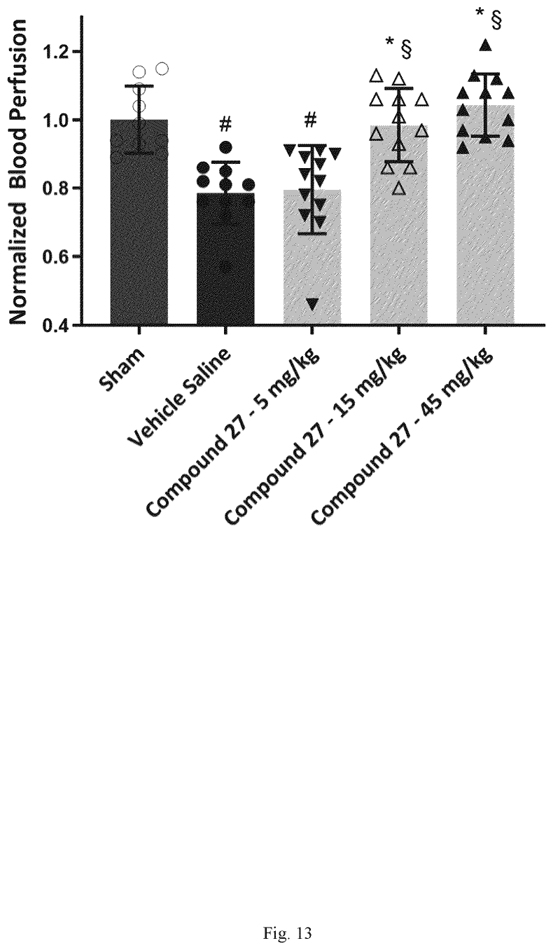

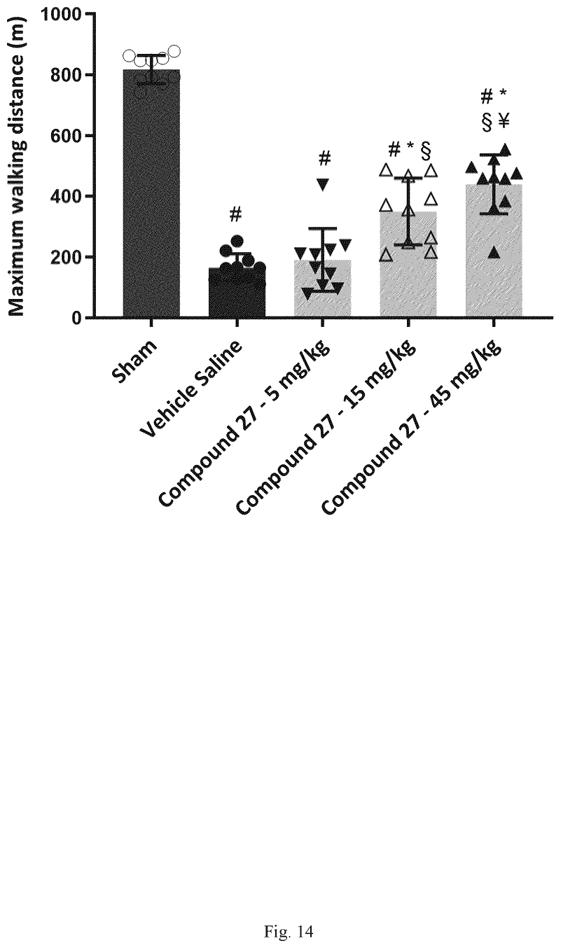

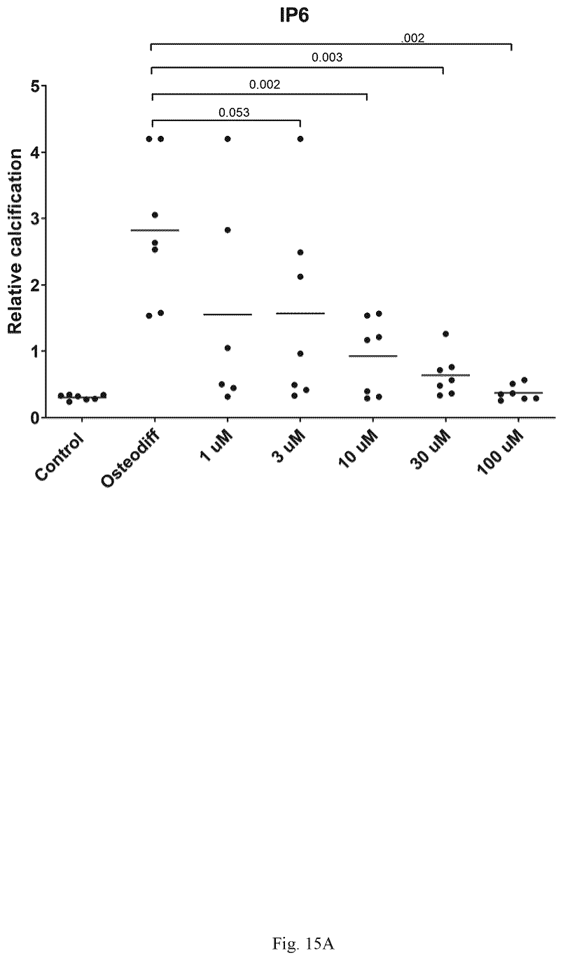

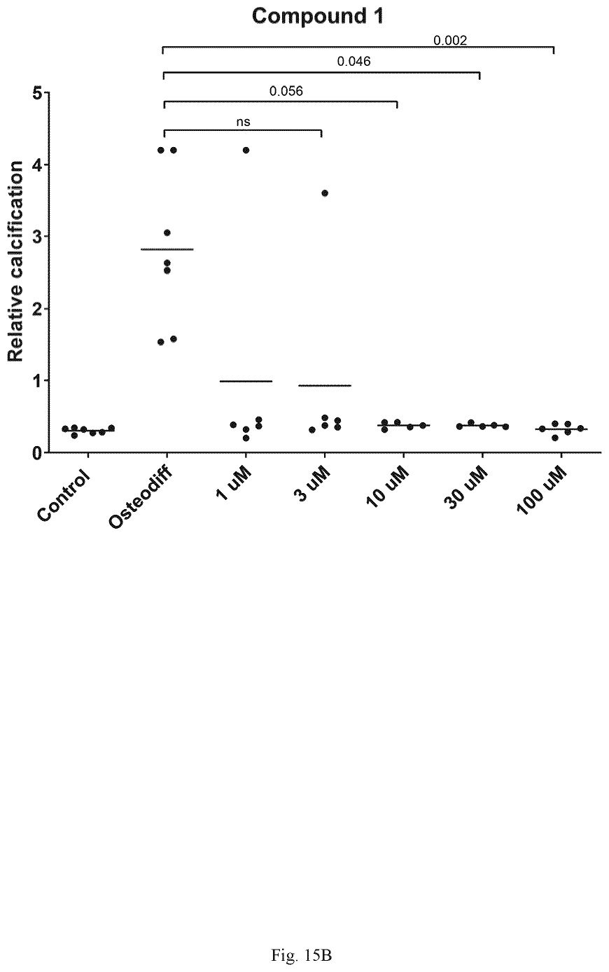

The inhibition in calcification in the femoral arteries (FIG. 12C), the effects on blood perfusion (FIG. 13) and the effects on maximum walking distance (FIG. 14) showed a dose-response effect from 5 mg/kg, with recovery of posterior limb blood perfusion at doses of 15 and 45 mg/kg. These results indicate that Compound 27 could be used in situations or conditions of atherosclerosis or arteriosclerosis. Arterial calcification leads to stiffening of the vessels, indicating the potential use of Compound 27 in arterial stiffness. Arterial stiffness in the lower limbs decreases blood supply leading to ischemic pain. The recovery in blood perfusion with Compound 27 indicates that it could be used to treat PAD, including limb ischemia or critical limb ischemia. The overall results in femoral calcification and blood perfusion, that lead to a functional improvement, as assessed by the maximum walking distance, indicate that Compound 27 could be used in peripheral vascular disease or peripheral arterial disease.

The overall effects on arterial calcification also indicates the potential use of Compound 27 in PXE, a genetic condition that leads to arterial calcification, amongst other locations.

### Example 56

### Prevention and Treatment of Calcinosis Cutis and Calciphylaxis

The dose-response efficacy of IP4-4,6 derivatives in the prevention and treatment of calcinosis cutis and calciphylaxis (CUA) induced by vitamin D₃ and FeCl₃ in a rat model was used. The model evaluated the effects of Compound 27 and IP6 on the formation of calcified skin plaques in rats for a duration of 8 days. Animals were sensitized at low concentration (LC, 35,000 IU/kg) and high concentration (HC, 50,000 IU/kg) doses of vitamin D₃. A FeCl₃ solution (0.5 mg/kg) was used as challenger to induce the formation of skin plaques. All animals were weighed every day before treatment.

Eighty-six male Sprague Dawley (SD) rats (Envigo Corp., Huntingdon, GB) weighing approximately 250-300 g were used. The animals were fed with a standard LASQCdiet® Rod14-H diet (LASvendi, Soest, DE). The animals were divided in 8 groups, 6 to 12 animals per group, as follows:

| | | Vit D | FeCl₃ | | Number |
|---|---|---|---|---|---|
| Group | Description | (IU/kg) | (mg/kg) | Dosage | of rats |
| 1 | Control Sham | Saline | Saline | — | 6 |
| 2 | Vehicle LC | 35,000 | 0.5 | NaCl 0.9% | 10 |
| 3 | Compound 27 | 35,000 | 0.5 | Compound 27 | 10 |
| | 60 mg/kg LC | | | 60 mg/kg | |
| 4 | Vehicle HC | 50,000 | 0.5 | NaCl 0.9% | 12 |
| 5 | IP6 | 50,000 | 0.5 | IP6 | 12 |
| | 60 mg/kg HC | | | 60 mg/kg | |
| 6 | Compound 27 | 50,000 | 0.5 | Compound 27 | 12 |
| | 30 mg/kg HC | | | 30 mg/kg | |
| 7 | Compound 27 | 50,000 | 0.5 | Compound 27 | 12 |
| | 60 mg/kg HC | | | 60 mg/kg | |
| 8 | Compound 27 | 50,000 | 0.5 | Compound 27 | 12 |
| | 100 mg/kg HC | | | 100 mg/kg | |

The following solutions were prepared: (a) 17,500 IU/mL (LC) and 25,000 IU/mL (HC) vitamin D₃ (cholecalciferol, Duphafral D₃ 1000; Zoetis Inc., Parsippany, NJ, US), (b) 0.3 mg/mL FeCl₃ (FeCl₃·6H₂O, 99%, Scharlab S. L., Barcelona, E S), (c) 30 mg/mL IP6 (660 g/mol phytic acid, pH 6.5-7.7), and (d) 15, 30, and 50 mg/mL Compound 27 (754.45 g/mol free acid, pH 6.5-7.7). All solutions were prepared in a 0.9% (w/v) physiological saline NaCl medium. An application volume of 2 mL/kg was employed.

Group 1—Control (sham), 6 animals. Animals in group 1 were administered subcutaneously physiological saline (0.9% (w/v) NaCl solution daily from D1 to D3 in the interscapular space, and with one saline 0.9% injection at each of the two ventral sites in the thorax on D4. Animals in group 1 were not administered Vit D or FeCl₃ solutions.

Group 2—Physiological saline solution low concentration (Vehicle LC): Vit D 35,000 IU/kg, 0.5 mg/kg FeCl₃ solution, 10 animals. Vit D at dose 35,000 IU/kg was administered subcutaneously x 3 consecutive days (D1, D2, D3), and one 0.5 mg/kg FeCl₃ injection at each of the two ventral sites in the thorax on D4. This group received a daily saline 0.9% s.c. treatment (D1 to D8).

Group 3—Compound 27 60 mg/kg LC: Vit D 35,000 IU/kg, 0.5 mg/kg FeCl₃, 10 animals. Vit D at dose 35,000 IU/kg was administered subcutaneously x 3 consecutive days (D1, D2, D3), and one 0.5 mg/kg FeCl₃ injection at each of the two ventral sites in the thorax on D4. This group received a daily Compound 27 s.c. treatment at 60 mg/kg dose (D1 to D8).

Group 4—Physiological saline solution high concentration (Vehicle HC): Vit D 50,000 IU/kg, 0.5 mg/kg FeCl₃, 12 animals. Vit D at dose 50,000 IU/kg was administered subcutaneously x 3 consecutive days (D1, D2, D3), and one 0.5 mg/kg FeCl₃ injection at each of the two ventral sites in the thorax on D4. This group received a daily saline 0.9% s.c. treatment (D1 to D8).

Group 5—IP6 60 mg/kg HC, Vit D 50,000 IU/kg, 0.5 mg/kg FeCl₃, 12 animals. Vit D at dose 50,000 IU/kg was administered subcutaneously x 3 consecutive days (D1, D2, D3), and one 0.5 mg/kg FeCl₃ injection at each of the two ventral sites in the thorax on D4. This group received a daily IP6 s.c. treatment at 60 mg/kg dose (D1 to D8).

Group 6—Compound 27 30 mg/kg HC, Vit D 50,000 IU/kg, 0.5 mg/kg FeCl₃, 12 animals. Vit D at dose 50,000 IU/kg was administered subcutaneously x 3 consecutive days (D1, D2, D3), and one 0.5 mg/kg FeCl₃ injection at each of the two ventral sites in the thorax on D4. This group received a daily Compound 27 s.c. treatment at 30 mg/kg dose (D1 to D8).

Group 7—Compound 27 60 mg/kg LC, Vit D 50,000 IU/kg, 0.5 mg/kg FeCl₃, 12 animals. Vit D at dose 50,000 IU/kg was administered subcutaneously x 3 consecutive days (D1, D2, D3), and one 0.5 mg/kg FeCl₃ injection at each of the two ventral sites in the thorax on D4. This group received a daily Compound 27 s.c. treatment at 60 mg/kg dose (D1 to D8).

Group 8—Compound 27 100 mg/kg HC, Vit D 50,000 IU/kg, 0.5 mg/kg FeCl₃, 12 animals. Vit D at dose 50,000 IU/kg was administered subcutaneously x 3 consecutive days (D1, D2, D3), and one 0.5 mg/kg FeCl₃ injection at each of the two ventral sites in the thorax on D4. This group received a daily Compound 27 s.c. treatment at 100 mg/kg dose (D1 to D8).

On D8, all animals were exsanguinated and then were sacrificed. Their necropsies were performed, and the skin tissue plaque formed at the FeCl₃ injection sites were collected for calcium content measurement. The tissues were cleaned with 0.15 M saline solution and lyophilized for 48-72 h and weighed. The lyophilized tissues were then digested using a 1:1 HNO₃:HClO₄ mixture in a dry bath incubator for 2-4 h at 180° C. The digested tissues were subsequently diluted using ultra-pure Milli-Q water (MilliporeSigma, Merck KGaA, Burlington, MA, US) to a final volume of 10 mL. Calcium content was quantified via inductively coupled plasma optical emission spectrometry (ICP-OES) using an Optima 7300 DV ICP-OES System spectrometer (PerkinElmer, Inc., Waltham, MA, US) according to the manufacturer's instructions.

The results show that the degree of inhibition reached by Compound 27 is higher than that reached by IP6 at the same dose. Moreover, Compound 27 inhibits the formation of skin plaque in rats in a dose-response manner indicating that Compound 27 could be used in conditions like calcinosis cutis and calciphylaxis. See Table 14. The effect on skin calcification also indicates the potential use of Compound 27 in PXE therapy, a genetic condition that leads to skin calcification, amongst other locations.

Statistical analysis: one-way ANOVA (p<0.01 for LC groups; p<0.0001 for HC groups) with Tukey post-hoc test. (#) significant differences vs Sham, (*) significant differences vs the corresponding Vehicle, (§) significant differences vs IP6 60 mg/kg (only applies to Compound 27 at the same dose), p<0.05.

TABLE 14

| | | Skin plaque |
|---|---|---|
| | | % inhibition of |
| Group | Description | calcification |
| 1 | Control Sham | 100 ± 0.04 |
| 2 | Vehicle LC | 0.00 ± 32.9 # |
| 3 | Compound 27 | 95.7 ± 0.9 * |
| | 60 mg/kg LC | |
| 4 | Vehicle HC | 0.00 ± 13.4 # |
| 5 | IP6 60 mg/kg HC | 58.8 ± 14.4 * |
| 6 | Compound 27 | 96.8 ± 0.7 * |
| | 30 mg/kg HC | |
| 7 | Compound 27 | 98.2 ± 0.1 *§ |
| | 60 mg/kg HC | |
| 8 | Compound 27 | 98.6 ± 0.1 * |
| | 100 mg/kg HC | |

### Example 57

### Treatment of Calcified Aortic Human Valves

The dose-response efficacy of IP4-4,6 derivatives in the treatment of calcified aortic human valves was analyzed using an in vitro model (Zabirnyk A, et al., Vasc Pharmacol 2019; 122-123, 106583, doi:10.1016/j.vph.2019.106583). The model evaluated the effects of IP6, Compound 1, Compound 3, and Compound 6 on the calcification of interstitial cells from aortic human valves (VIC).

Human calcified aortic valve leaflets were obtained during aortic valve replacement and were harvested. After the removal of the valve endothelial cells, VIC were isolated. The leaflets were placed in 30 mL of basic growth medium (DMEM), supplemented with 10% fetal bovine serum (FBS) and 50 μg/mL gentamycin with the addition of 1 mg/mL collagenase II. The cells were incubated overnight at 37° C. with 5% CO₂. Next day, the digested tissue was homogenized by pipetting with a serological pipet and centrifuged at 300 g for 5 min and the supernatant carefully removed by aspiration.

The pellet was washed in 10 mL of fresh DMEM and centrifuged as described previously. The supernatant was removed by aspiration, the cell pellet was resuspended in basic growth media and seeded in 75 cm³ flasks. VIC were cultured at 37° C. and 5% CO₂ with growth media changed twice a week until they reached ~90% confluency. Cells were passaged at ratio of 1:2, propagated and frozen.

### 1. VIC Osteogenic Differentiation and Inhibition

For osteogenic differentiation, VIC from calcified human aortic valves (n=7) were seeded in 24-well tissue culture plates at 30×10⁴ cells/well in basic growth medium and cultured overnight at 37° C., 5% CO₂. The following day, osteogenic differentiation was induced by supplementing the medium with 50 μM ascorbic acid, 0.1 μM dexamethasone and 10 mM β-glycerophosphate. Osteogenic medium was changed twice a week for three weeks. The negative control group (no induction of calcification) was cultivated in basic growth media for the same period of time and media was changed following the same protocol.

The concentration-response study was performed with the same VIC donors in all groups (n=7, per concentration and test item). Increasing concentrations (1, 3, 10, 30, and 100 μM) of test items Compound 1, Compound 3, and Compound 6 were incubated starting at week 1 when the osteogenic differentiation was induced and compared with the activity of IP6. A positive control group (osteogenic medium with no inhibitors added) was performed using VIC from the same donors.

### 2. Calcium Staining and Quantification

Alizarin Red staining was performed to assess calcification. The cell medium was removed, the cells were washed with PBS and fixed with 70% ethanol for 1 h at room temperature. Then, cells were washed with Milli-Q water and stained with Alizarin Red according to the manufacturer's instructions. To quantify calcium accumulation, Alizarin Red staining was extracted and measured spectrophotometrically. Briefly: for 24-well plate, 200 μL 10% acetic acid was added to each well and incubated for 30 min at room temperature with gentle agitation. After that, cells were detached using a cell scraper and the resulting suspension was transferred to a 1.5 mL microcentrifuge tube and vortexed vigorously for 30 s. The cells were then heated to 85° C. for 10 min before the tubes were transferred to ice for 5 min and chilled. Then, the cells were centrifuged at 15000 g for 15 min. The supernatant was transferred to a new 1.5 mL microcentrifuge tube, 75 μL of 1M NaOH were added to each tube to achieve a pH between 4.1 and 4.5. Finally, 50 μL from the tube were transferred to a clear bottom 96-well plate and the absorbance at 405 nm was measured on a plate reader.

Statistics were analyzed by GraphPad Prism (version 9, Dotmatics, Boston, MA, US). The VIC cultures which failed to differentiate and calcify under osteodifferentiation media were excluded. Normality of the data was assessed using a Shapiro-Wilk test. For results that were normally distributed, a one-way ANOVA for normal distribution with DMS post-test was applied. For the results that were not normally distributed, a nonparametric one-way ANOVA (Kruskal-Wallis test) with Dunn's post-test was applied. Relative calcification results are shown as dot plots and mean. A value of p<0.05 was considered statistically significant.

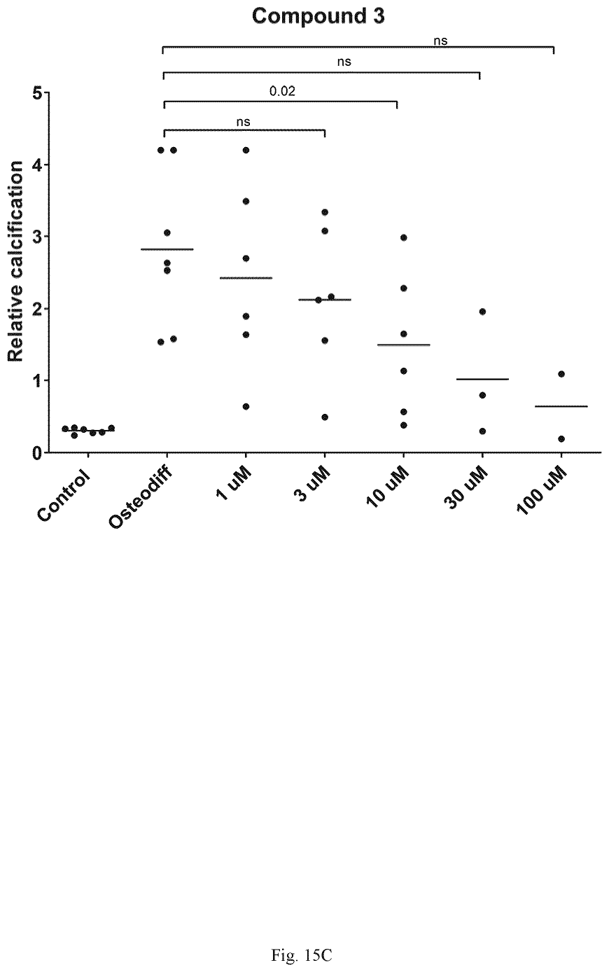

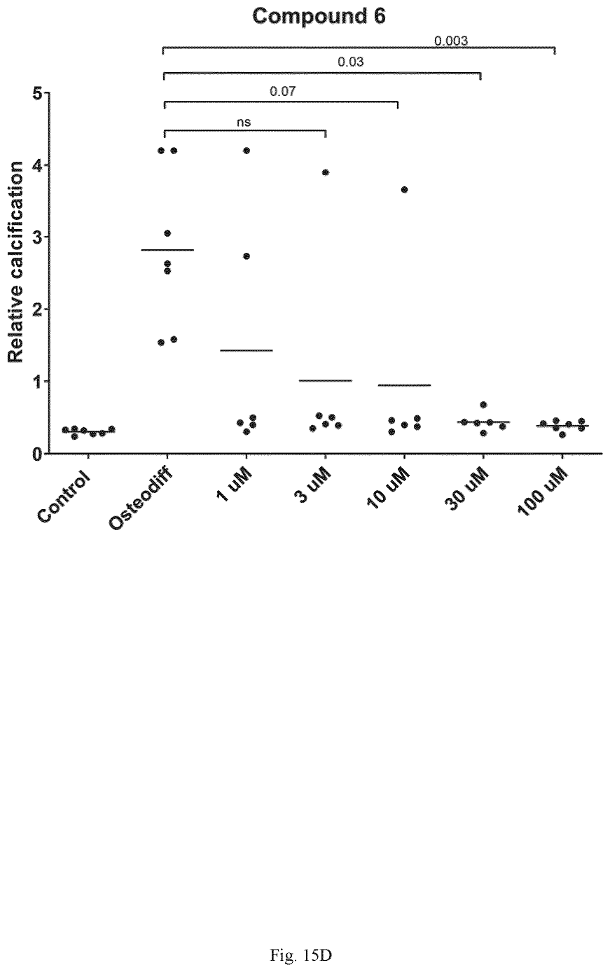

The results show that IP4-4,6 derivatives Compound 1, Compound 3, and Compound 6 were effective in inhibiting the calcification of VIC in vitro in a dose-response manner. See FIG. 15A to 15D. These results indicate that these compounds could be used in situations or conditions like valvular calcification, valve stenosis or calcific aortic valve stenosis. Calcified valves can lead to heart hypertrophy, indicating that these compounds could be used to treat or prevent left ventricular hypertrophy. Ultimately, heart hypertrophy may lead to arrhythmias, heart failure, congestive heart failure, cardiac disease, cardiovascular mortality or cardiac death, indicating that these compounds could be used in these situations or conditions.

### Example 58

### Treatment of Pseudoxanthoma elasticum

A stable CRISPR/Cas9 abcc6a^{-/-} zebrafish model characterized for spinal column hypermineralization was used for assaying the efficacy of Compound 27 in the treatment of Pseudoxanthoma elasticum (PXE; Van Gils M, et al., J Invest Dermatol 2022; 138:2333-2342).

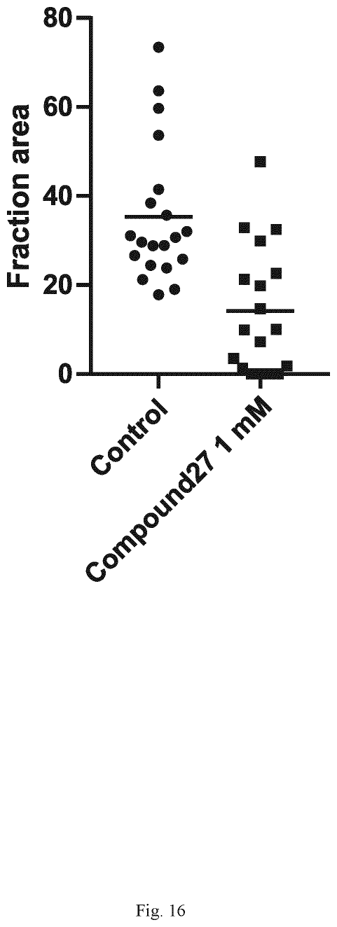

Abcc6a^{-/-} larvae were treated with Compound 27 at different concentrations (i.e., 100 μM, 300 μM, and 1 mM) for 7 days, starting at 3 days post fertilization (dpf), with daily refreshment of the medium containing the solved compounds. On 10 dpf, the larvae were euthanized and stained with Alizarin Red to visualize the spinal column hypermineralization, using a Leica M165 FC microscope (Leica Microsystems GmbH, Wetzlar, DE). Semi-quantification of calcification was done through image processing with ImageJ image processing with ImageJ (Rasband W, Research Services Branch, NIH, Bethesda, MD, US). The results revealed that treatment with Compound 27 at 1 mM significantly reduced the spinal column hypermineralization in abcc6a−/− larvae on average with 60% (relative calcified area 35.2 vs 14.17; p<0.001) with conservation of the physiological calcification and bone formation and hence the potential of Compound 27 in PXE therapy. See FIG. 16.

It is to be appreciated that the Detailed Description section, and not the Summary and Abstract sections, is intended to be used to interpret the claims. The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described examples but should be defined only in accordance with the following claims and their equivalents.

1. Compound of general formula I: 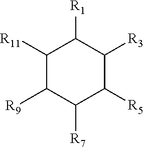
a pharmaceutically acceptable salt thereof, or a combination thereof, wherein
(i) R₁, R₃, R₇, and R₁₁ independently represent OPO₃²⁻; R₅ and R₉ are substituent groups each one corresponding to the formula —O-(alkyl)ₙ-X, wherein n is an integer between 1 and 20, wherein the terminal group X is selected from the group consisting of —H, —OR, —NRR′, —COOR, —CONRR′, —NHCOR, —NHCOOR, —OCONR, —NHSO₂R, —NHCONRR′, halogen, —CF₃, alkyl, alkenyl, alkynyl, carbocycle, and heterocycle, and wherein R and R′ are H or an alkyl group;
(ii) R₁, R₃, R₇, and R₁₁ independently represent OPO₃²⁻; R₅ and R₉ are substituent groups each one corresponding to the formula —O-(alkyl)_{y}-Cy-(alkyl)_{y′}-Z, wherein y and y′ are an integer between 0 and 10, wherein Cy is a cyclic linker, wherein the terminal group Z is selected from the group consisting of alkyl, —COR, —OR, —NRR′, —COOR, —CONRR′, —NHCOR, —NHCOOR, —OCONR—NHSO₂R, —NHCONRR′, halogen, and —CF₃, and wherein R and R′ are H or an alkyl group;
(iii) R₁, R₃, R₇, and R₁₁ independently represent OPO₃²⁻; R₅ and R₉ are substituent groups each one corresponding to the formula —O-(alkyl)_{y}-A-(alkyl)_{y′}-Z′, wherein y and y′ are an integer between 0 and 10, wherein A is a linker selected from the group consisting of —CONR—, —NHCOO—, —NHSO₂, —NHCONR—, —NHCO—, and —OCONR, wherein the terminal group Z′ is selected from the group consisting of —OR, —NRR′, —COOR, —CONRR′, —NHCOR, —NHCOOR, —OCONR, —NHSO₂R, —NHCONRR′, carbocycle, heterocycle, and wherein R and R′ are H or an alkyl group; and
(iv) the compound of formula I is an analog of (i), (ii) or (iii) wherein at least one of R₁, R₃, R₇ or R₁₁ is thiophosphate (—OPSO₂²⁻) for use in the treatment, inhibition of progression, and prevention of ectopic calcification or the consequences thereof in a subject in need thereof.

2. Compound for use according to claim 1, wherein the compound is a compound of general formula II, III, IV, V, VI, VII, VIII, IX, X, XI, XII, XIII or XIV, and any combination thereof.

3. Compound for use according to any one of claims 1 to 2, wherein the compound is selected from the group consisting of Compound 1 to Compound 53, and any combination thereof.

4. Compound for use according to claim 3, wherein the compound is a Compound 1 of chemical structure:

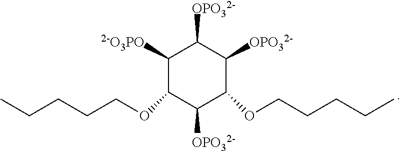

5. Compound for use according to claim 3, wherein the compound is a Compound 3 of chemical structure:

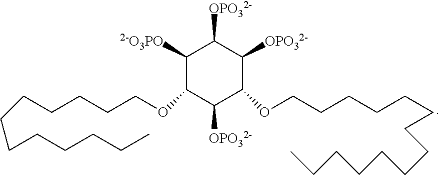

6. Compound for use according to claim 3, wherein the compound is a Compound 6 of chemical structure:

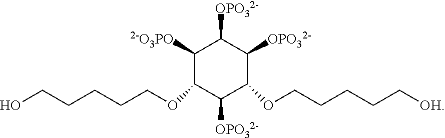

7. Compound for use according to claim 3, wherein the compound is a Compound 27 of chemical structure:

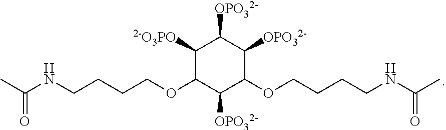

8. Pharmaceutical composition for use in the treatment, inhibition of progression, or prevention of ectopic calcification or the consequences thereof in a subject in need thereof comprising a compound for use according to any one of claims 1 to 7 and any combination thereof, and at least one pharmaceutically acceptable excipient or carrier.

9. Compound or pharmaceutical composition for use according to any of the preceding claims, wherein a therapeutically effective amount of the compound or pharmaceutical composition is administered to the subject in need thereof.

10. Compound or pharmaceutical composition for use according to any of the preceding claims, comprising the treatment, inhibition of progression, and prevention of a disease and/or condition related to ectopic calcification or the consequences thereof in a subject in need thereof.

11. Compound or pharmaceutical composition for use according to claim 10, wherein the ectopic calcification or the consequences thereof comprise adrenal and intracranial calcification in familial cerebral cavernous malformations, adynamic bone, age-related macular degeneration (AMD) related to calcium deposits, bone cancer, bone mineral disease, breast calcification, calcific band keratopathy, calcific tendinitis, calcification in osteoarthritis, calcification of articular cartilage in osteoarthritis, calcification of joints and arteries (CALJA), calcification of the seminal vesicles, calcinosis cutis, calciphylaxis (CUA), calcium pyrophosphate deposition disease (CPPD), cardiovascular diseases or associated conditions, chondrocalcinosis, colon cancer, diabetic kidney disease, dystrophic calcification, failure of renal transplant grafts, familial cerebral cavernous malformations (FCCM), fibrodysplasia ossificans progressiva (FOP), hyperostosis-hyperphosphatemia syndrome (HHS), hyperphosphatemic familial tumoral calcinosis (HFTC), idiopathic brain calcification (Fahr's disease), idiopathic mesenteric phlebosclerosis (IMP), kidney stones (i.e., renal lithiasis), metastatic calcification, nephrocalcinosis, neurocysticercosis-related calcification, osteomalacia, osteoporosis, pineal calcification, phlebosclerotic colitis, podagra, primary familial brain calcification (PFBC), primary hyperoxaluria (PH), pseudoxantoma elasticum (PXE), rheumatoid arthritis, sialolithiasis, Sjorgen's syndrome parotid glands calcification, seminal vesicle calculi, skin cancer, soft tissue calcification owing to sarcoidosis, Wolman's disease adrenal calcification, and wound healing related to diabetic ulcers.

12. Compound or pharmaceutical composition for use according to claim 11, wherein the cardiovascular diseases or associated conditions comprise acute ischemic stroke (ACS), aneurysm, angina pectoris (chronic stable angina), aortic artery calcification, aortic calcification, aortic stenosis, aortic valve calcification, arrhythmia, arteriosclerosis, arterial stiffness, arteriovenous fistula (AVF) failure, atherosclerosis, calcific aortic valve stenosis, cardiac death, cardiac disease, cardiovascular calcification, cardiovascular disease in chronic kidney disease (CKD) patients, cardiovascular disease linked to aging, cardiovascular mortality, cerebrovascular disease, congestive heart failure, coral reef aorta (CRA), coronary artery calcification, coronary artery disease, coronary disease, critical limb ischemia, electrocardiogramormalities, general arterial calcification of infancy (GACI), heart failure, hypertension, ischaemia, left ventricular hypertrophy, major adverse cardiovascular events (MACE) in hemodialysis (HD) patients, Mönckeberg's medial sclerosis (MMS), myocardial calcification, myocardial infarction, myocardial ischemia, pericardial calcification, peripheral arterial disease (PAD), peripheral vascular disease (PVD), porcelain aorta and calcification of anastomosis site after coronary artery bypass grafting (CABG), portal vein calcification, stroke, thrombosis, valvular calcification, and vascular calcification.

13. Compound or pharmaceutical composition for use according to any of the preceding claims, wherein the subject is with kidney failure.

14. Compound or pharmaceutical composition for use according to any of the preceding claims, wherein the subject is on dialysis.

15. Compound or pharmaceutical composition for use according to any of the preceding claims, wherein the administration is topical, enteral or parenteral.

16. Compound or pharmaceutical composition for use according to claim 15, wherein the parenteral administration is intravenous, subcutaneous, intramuscular or by intravenous infusion.

17. Compound or pharmaceutical composition for use according to claim 16, wherein the intravenous infusion is administered using a dialysis apparatus.

18. Compound or pharmaceutical composition for use according to any of the preceding claims, wherein the subject is human.

19. A kit or article of manufacture comprising at least one compound or pharmaceutical composition for use according to any one of the preceding claims, and any combination thereof.
